(19) United States
(12) Patent Application Publication
(10) Pub. No.: US 2025/0313837 A1
(43) Pub. Date: Oct. 9, 2025

(54) PRODUCTS AND COMPOSITIONS

(71) Applicant: Sirnaomics, Inc., Germantown, MD (US)

(72) Inventor: Dmitry SAMARSKY, Germantown, MD (US)

(21) Appl. No.: 18/982,618

(22) Filed: Dec. 16, 2024

Related U.S. Application Data
(63) Continuation of application No. PCT/US23/68523, filed on Jun. 15, 2023.
(60) Provisional application No. 63/352,503, filed on Jun. 15, 2022.
(60) Provisional application No. 63/408,318, filed on Sep. 20, 2022.

(51) Int. Cl. C12N 15/113

(52) U.S. Cl. CPC C12N 15/113; C12N 2310/14; C12N 2310/351; C12N 2310/531

(57) ABSTRACT

The present disclosure relates to products, and compositions, and their uses. In particular, the present disclosure relates to nucleic acid products that modulate, in particular interfere with or inhibit C5 gene expression. The products can be oligomeric compounds that comprise at least a first region of linked nucleosides having at least a first nucleobase sequence that is at least partially complementary to at least a portion of RNA transcribed from a C5 gene.

### RELATED APPLICATIONS

The present application is a continuation of International Application No. PCT/US2023/068523, filed on Jun. 15, 2023, which claims the benefit of and priority to U.S. Provisional Patent Application No. 63/352,503, filed on Jun. 15, 2022 and U.S. Provisional Patent Application No. 63/408,318, filed on Sep. 20, 2022, both of which are incorporated herein by reference in their entireties.

### SEQUENCE LISTING

The instant application contains a sequence listing which has been submitted electronically in XML format and is hereby incorporated by reference in its entirety. Said XML document, created on Jun. 8, 2023, is named 4690_0070I_SL and is 4587 kilobytes in size.

### FIELD

The present disclosure relates to products, compositions, and their uses. In particular, the present disclosure relates to nucleic acid products that modulate, in particular interfere with or inhibit, complement component C5 gene expression. Embodiments of the present disclosure can therefore provide methods, compounds, and compositions for reducing expression of C5 mRNA and protein in an animal. Such methods, compounds, and compositions are useful to treat, prevent, or ameliorate complement system-associated including C5-associated disorders such as paroxysmal nocturnal hemoglobinuria (PNH), Alzheimer's disease, Atherosclerosis, Inflammation of the choroid plexus, Generalized myasthenia gravis (gMG), amyotrophic lateral sclerosis (ALS), Lupus nephritis (LN), Central nervous system (CNS) diseases; Age-related macular degeneration (AMD) and/or Geographic atrophy (GA); Uveitis and/or panuveitis; Cold agglutinin disease, Membranoproliferative glomerulonephritis (MPGN), Guillain-Barré syndrome, Shiga toxin-producing E. coli hemolytic-uremic syndrome (STEC-HUS) and organ transplantation-associated autoimmune diseases.

### BACKGROUND

The complement system is part of the innate immune system. Compared to the adaptive immune system, it is evolutionary older and conserved across most taxa. Its function includes decorating microbes of potentially pathogenic nature (a process referred to as opsonization) and target them for destruction, which is effected by a macromolecular assembly known as the membrane attachment complex (MAC). Certain components of the complement system, once activated, contribute to chemoattraction and activation of leukocytes.

Complement activation may be triggered by various factors, which all involve presence of microbes but may also involve components of the adaptive immune system such as Ig including IgM. Three main pathways of complement activation have been recognized and are referred to as classical pathway, alternative pathway and lectin pathway.

In functional terms, complement activation occurs inherently at a low level (spontaneous cleavage of C3 to yield C3a and C3b) and is reinforced in the presence of microbes via an enzymatic cascade converting inactive forms of enzymes (zymogenes) into their active counterparts. The term “convertase”, such as C3 convertase, is primarily a functional term and may refer to structurally distinct complexes. One type of C3 convertases is a complex of C3b and complement factor B (CFB, Factor B). Once formed, a C3 convertase can convert large amounts of C3 into its cleavage products C3a and C3b within short amount of time. The specific C3 convertase, which is a complex of C3b and Factor B, has originally been described in the context of the alternative pathway, but may form also in the context of the other two pathways. Within the alternative pathway, Factor B is also a constituent of C5 convertase, a complex which converts C5, a more downstream component of the pathway, into its active form. The formation of C5 by C5 convertase, cleaving C5 into C5b and C5a is the initiating event in the late steps of complement activation. Based on C5b, the membrane attack complex (MAC) is formed which lyses a target membrane by building a pore out of C9 molecules.

### Disease

The complement system is generally triggered by patterns of binding sites on surfaces. These binding sites may be constituents of a microbe or pathogen, but may also be antibodies which previously bound to any target. In the latter case, the complement system acts to reinforce the adaptive immune system. As a consequence, and in case the mentioned antibodies are autoantibodies, the complement system exacerbates an undesirable autoimmune reaction. Interfering with the complement system in such a setting is a means to treat or ameliorate autoimmune diseases. Since the complement system, more specifically C1 of the classical pathway recognizes the constant portions of antibodies, interfering with the complement system opens an avenue to generally interfering with auto-immune disorder without particular limitation. Having the that, experience tells that autoimmune disorders affect skin, joints and kidneys more frequently than other organs.

On the other hand, complement dysfunction, in the absence of autoantibodies may be a trigger of disorders as well. In addition, in this context it applies that, owing to the generic mechanism of the complement system, the disease amenable to treatment by an inhibitor of the complement system, more specifically by an inhibitor of complement component C5 is not particularly limited.

### Treatment

Eculizumab is a humanized monoclonal antibody targeting C5 and has been approved for PNH treatment. A murine cell line is used for its production. Eculizumab shall not be used in patients with sensitivity against murine proteins. Treatment with eculizumab is very expensive and costs may exceed 600000 EUR per year and patient.

There therefore remains a need for therapies to treat complement-associated diseases including complement component C5-associated diseases. We, therefore, aim to provide compounds, methods, and pharmaceutical compositions for the treatment of such diseases.

Double-stranded RNA (dsRNA) able to complementarily bind expressed mRNA has been shown to be able to block gene expression (Fire et al., 1998, Nature. 1998 Feb. 19; 391 (6669):806-1 1 and Elbashir et at., 2001, Nature. 2001 May 24; 41 1 (6836):494-8) by a mechanism that has been termed RNA interference (RNAi). Short dsRNAs direct gene-specific, post-transcriptional silencing in many organisms, including vertebrates, and have become a useful tool for studying gene function. RNAi is mediated by the RNA-induced silencing complex (RISC), a sequence-specific, multi-component nuclease that destroys messenger RNAs homologous to the silencing trigger loaded into the RISC complex. Interfering RNA (IRNA) such as siRNAs, antisense RNA, and micro-RNA are oligonucleotides that prevent the formation of proteins by gene-silencing i.e., inhibiting gene translation of the protein through degradation of mRNA molecules. Gene-silencing agents are becoming increasingly important for therapeutic applications in medicine.

According to Watts and Corey in the Journal of Pathology (2012; Vol 226, p 365-379) there are algorithms that can be used to design nucleic acid silencing triggers, but all of these have severe limitations. It may take various experimental methods to identify potent siRNAs, as algorithms do not take into account factors such as tertiary structure of the target mRNA or the involvement of RNA binding proteins. Therefore, the discovery of a potent nucleic acid silencing trigger with minimal off-target effects is a complex process. For the pharmaceutical development of these highly charged molecules, it is necessary that they can be synthesised economically, distributed to target tissues, enter cells and function within acceptable limits of toxicity. An aim is to, therefore, provide compounds, methods, and pharmaceutical compositions for the treatment of C5-related diseases as described herein, which comprise oligomeric compounds that modulate, in particular inhibit, gene expression by RNAi.

### SUMMARY

The aforementioned problem of providing compounds and treatments having the potential of efficiently reducing the effects of C5-related diseases is solved by the present disclosure.

According to a first aspect, the present disclosure is directed to an oligomeric compound capable of inhibiting expression of complement component C5, wherein the compound comprises at least a first region of linked nucleosides having at least a first nucleobase sequence that is at least partially complementary to at least a portion of RNA transcribed from an C5 gene, wherein the first nucleobase sequence is selected from the following sequences, or a portion thereof: sequences of Table 1a (SEQ ID NOs: 1 to 250), wherein the portion optionally has a length of at least 18 nucleosides.

Particularly optional embodiments according to the first aspect of the present disclosure relate to optimized hairpin RNAs (referred to as mxRNAs); for further details see the embodiments and their discussion further below.

Furthermore, and as disclosed further below, the disclosure also relates to double-stranded RNAs (dsRNAs). Deviant from mxRNAs, dsRNAs lack a loop connecting antisense and sense portions and therefore comprise two strands. The two strands are not covalently connected to each other but form a duplex region where base pairing occurs.

According to a second aspect, the present disclosure is directed to nucleic acid construct comprising at least:
(a) a first nucleic acid portion that is at least partially complementary to at least a first portion of an RNA, which is transcribed from a C5 gene;
(b) a second nucleic acid portion that is at least partially complementary to at least a second portion of an RNA, which is transcribed from a C5 gene, the second portion being different from the first portion;
(c) a third nucleic acid portion that is at least partially complementary to the first nucleic acid portion of (a), so as to form a first nucleic acid duplex region therewith;
(d) a fourth nucleic acid portion that is at least partially complementary to the second nucleic acid portion of (b), so as to form a second nucleic acid duplex region therewith.

Optional and/or exemplary features of constructs according to the second aspect of the present disclosure are as follows:
1) they contain multiple (2 or more) at least partially double-stranded agents capable of triggering RNA interference, tied together into a single nanostructure predominantly through complementary (Watson-Crick) interactions;
2) optionally, other (e.g.) covalent bindings may be used to build the constructs and/or add various ligands (e.g., delivery/targeting moieties such as GalNAc and or other carbohydrates, cholesterol, peptides, or small molecules, optionally attached via linkers);
3) the constructs of the disclosure predominantly comprise chemically modified nucleotides (e.g., 2′F, 2′OMe, LNO, PNA, MOE, BNA, PMO, phosphorothioate, phosphorodithioate, etc.), mostly (but not only) to increase resistance to nucleases;
4) the constructs contain “fragile” components (e.g. chemical linkers, unmodified nucleotides, etc.), which allow the constructs to disassemble upon exposure to certain biologic environments (e.g. exposure to extra- and/or intra-cellular fluids); particular examples could be (but not limited): a) cleavage of the oligo backbone by nucleases in the sites with non-modified nucleotides; b) cleavage of the chemical linkage due to the change of pH (e.g. in endosomes);
5) disassembly upon exposure to the certain biologic environments releases the active components (e.g., the at least partially double-stranded agents capable of triggering RNA interference) to modulate (up- or down-regulate, optionally down-regulate) target gene expression in cells/organisms;
6) the constructs can be used to modulate, optionally down-regulate or silence gene expression, to study gene function, or to treat various diseases associated with the target genes to be down regulated.

According to a third aspect, the present disclosure is directed to a composition comprising an oligomeric compound according to the first aspect and/or a nucleic acid construct according to the second aspect, and a physiologically acceptable excipient.

According to a fourth aspect, the present disclosure is directed to a pharmaceutical composition comprising an oligomeric compound according to the first aspect and/or a nucleic acid construct according to the second aspect.

According to a fifth aspect, the present disclosure is directed to an oligomeric compound according to the first aspect and/or a nucleic acid construct according to the second aspect, for use in human or veterinary medicine or therapy.

According to a sixth aspect, the present disclosure is directed to an oligomeric compound according to the first aspect and/or a nucleic acid construct according to the second aspect, for use in a method of treating a disease or disorder.

According to a seventh aspect, the present disclosure is directed to a method of treating a disease or disorder comprising administration of an oligomeric compound according to the first aspect and/or a nucleic acid construct according to the second aspect, to an individual in need of treatment.

According to an eighth aspect, the present disclosure is directed to a use of an oligomeric compound according to the first aspect or according to the second aspect, for use in research as a gene function analysis tool.

According to a ninth aspect, the present disclosure is directed to a use of an oligomeric compound according to the first aspect or the second aspect in the manufacture of a medicament for a treatment of a disease or disorder.

### Effects Achieved by the Oligomeric Compounds

Due to the use of the oligomeric compounds according to the present disclosure, a significant reduction of gene expression of complement component C5 in vitro and in vivo is achieved as e.g., shown in the examples disclosed herein. The most inhibiting compounds surprisingly produce knockdowns of up to 70 to 75% C5 mRNA reduction in vitro. Furthermore, large amounts of compounds are also capable of producing knockdowns of 60 to 70% reduction of C5 mRNA in vitro. The significant in vitro activity of these compounds could also be confirmed within in vivo studies disclosed herein. Therefore, the oligomeric compounds of the present disclosure are suitable candidates for the treatment of C5-related diseases.

Furthermore, it was surprisingly found that, in certain embodiments, the mentioned effects are achieved by using oligomeric compounds according to the present disclosure for inhibiting the expression of C5 in the form of shRNA constructs having a reduced length of e.g., 33 nucleosides (also called “mxRNA”) compared to conventional shRNA molecules having greater lengths. This can e.g., make a synthesis of shRNA molecules more efficient, because less units are needed.

For certain oligomeric compounds according to the present disclosure, being in the form of shRNA constructs for inhibiting the expression of C5, it was surprisingly found out that the aforementioned effects can be achieved by using short sense strands within the shRNA having a length of optionally 14 nucleosides which is shorter than the length of the sense strands in conventional shRNA molecules.

Due to their successful C5 knockdown of the inventive compounds in their mxRNA form it is also plausible that they are functioning, the same way as a part of muRNA constructs as disclosed herein. This is in particular because, without wishing to be bound by a particular theory, it is assumed that the active species are the same.

### BRIEF DESCRIPTION OF THE FIGURES

FIG. 1 shows single dose curves of certain C5 mxRNA compounds of the present disclosure and their activity in inhibiting C5 gene expression (primary screening).

FIG. 2 shows dose curves of 25 C5 mxRNA compounds and their activity in inhibiting C5 gene expression (secondary screening).

FIG. 3 shows dose curves of C5 mxRNA lead compounds for preparation in vivo and their dose curves.

FIG. 4 shows a study schedule and study information for a study relating to C5 targeting mxRNA leads for candidate dose and duration response study in humanized liver-uPA-SCID mice (PXB) model.

FIG. 5 shows results of the C5 targeting mxRNA construct study for dose and duration response in humanized liver-uPA-SCID mice (PXB).

FIG. 6 shows a study schedule and study information for a study relating to an evaluation of a duration effect of human complement C5 targeting mxRNA, in the humanized liver-uPA-SCID mouse model.

FIG. 7 shows results of the study of an evaluation of a duration effect of human complement C5 targeting mxRNA, in the humanized liver-uPA-SCID mouse model.

### DETAILED DESCRIPTION AND EMBODIMENTS

Further embodiments (items) of the present disclosure are described below by way of example only. These examples represent the best ways of putting the disclosure into practice that are currently known to the applicant although they are not the only ways in which this could be achieved.

It will be understood that the benefits and advantages described herein may relate to one embodiment or may relate to several embodiments. The embodiments are not limited to those that solve any or all of the stated problems or those that have any or all of the stated benefits and advantages. Embodiments labelled “optional” or “optionally” are not intended to limit the scope of the claims but to show optional embodiments of the present disclosure.

Features of different aspects and embodiments of the disclosure may be combined as appropriate, as would be apparent to a skilled person, and may be combined with any of the aspects of the disclosure.

### Definitions

The following definitions pertain to the disclosure throughout. In many instances, the definitions, in addition to the respective definition as such, provide non-exhaustive listings of possible implementations, which amount to optional embodiments.

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 21st edition, 2005; and “Antisense Drug Technology, Principles, Strategies, and Applications” Edited by Stanley T. Crooke, CRC Press, Boca Raton, Florida; and Sambrook ct al., “Molecular Cloning, A laboratory Manual,” 2nd Edition, Cold Spring Harbor Laboratory Press, 1989, which are hereby incorporated by reference for any purpose. Where permitted, all patents, applications, published applications and other publications and other data referred to throughout in the disclosure are incorporated by reference herein in their entirety.

Unless otherwise indicated, the following terms have the following meanings:

As used herein, “excipient” means any compound or mixture of compounds that is added to a composition as provided herein that is suitable for delivery of an oligomeric compound.

As used herein, “nucleoside” means a compound comprising a nucleobase moiety and a sugar moiety. Nucleosides include, but are not limited to, naturally occurring nucleosides (as found in DNA and RNA) and modified nucleosides. Nucleosides may be linked to a phosphate moiety, phosphate-linked nucleosides also being referred to as “nucleotides”. The structural features and/or the lengths of oligomeric compounds or nucleic acid constructs disclosed herein is expressed in terms of “nucleosides” or “nucleotides”.

As used herein, “chemical modification” or “chemically modified” means a chemical difference in a compound when compared to a naturally occurring counterpart. Chemical modifications of oligonucleotides include nucleoside modifications (including sugar moiety modifications and nucleobase modifications) and internucleoside linkage modifications. In reference to an oligonucleotide, chemical modification does not include differences only in nucleobase sequence.

As used herein, “furanosyl” means a structure comprising a 5-membered ring comprising four carbon atoms and one oxygen atom.

As used herein, “naturally occurring sugar moiety” means a ribofuranosyl as found in naturally occurring RNA or a deoxyribofuranosyl as found in naturally occurring DNA. A “naturally occurring sugar moiety” as referred to herein is also termed as an “unmodified sugar moiety”. In particular, such a “naturally occurring sugar moiety” or an “unmodified sugar moiety” as referred to herein has a —H (DNA sugar moiety) or —OH (RNA sugar moiety) at the 2′-position of the sugar moiety, especially a —H (DNA sugar moiety) at the 2′-position of the sugar moiety.

As used herein, “sugar moiety” means a naturally occurring sugar moiety or a modified sugar moiety of a nucleoside. As used herein, “modified sugar moiety,” means a substituted sugar moiety or a sugar surrogate.

As used herein, “substituted sugar moiety” means a furanosyl that has been substituted. Substituted sugar moieties include, but are not limited to, furanosyls comprising substituents at the 2′-position, the 3′-position, the 5′-position and/or the 4′-position. Certain substituted sugar moieties are bicyclic sugar moieties.

As used herein, “2′-substituted sugar moiety” means a furanosyl comprising a substituent at the 2′-position other than H or OH. Unless otherwise indicated, a 2′-substituted sugar moiety is not a bicyclic sugar moiety (i.e., the 2′-substituent of a 2′-substituted sugar moiety does not form a bridge to another atom of the furanosyl ring).

As used herein, “MOE” means —OCH2CH2OCH3.

As used herein, “2′-F nucleoside” refers to a nucleoside comprising a sugar comprising fluorine at the 2′ position. Unless otherwise indicated, the fluorine in a 2′-F nucleoside is in the ribo position (replacing the OH of a natural ribose). Duplexes of uniformly modified 2′-fluorinated (ribo) oligonucleotides hybridized to RNA strands are not RNase H substrates while the analogues retain RNase H activity.

As used herein the term “sugar surrogate” means a structure that does not comprise a furanosyl and that is capable of replacing the naturally occurring sugar moiety of a nucleoside, such that the resulting nucleoside sub-units are capable of linking together and/or linking to other nucleosides to form an oligomeric compound which is capable of hybridizing to a complementary oligomeric compound. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholinos, cyclohexenyls and cyclohexitols.

As used herein, “bicyclic sugar moiety” means a modified sugar moiety comprising a 4 to 7 membered ring (including but not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments, the 4 to 7 membered ring is a furanosyl. In certain such embodiments, the bridge connects the 2′-carbon and the 4′-carbon of the furanosyl.

As used herein, “nucleotide” means a nucleoside further comprising a phosphate linking group. As used herein, “linked nucleosides” may or may not be linked by phosphate linkages and thus includes, but is not limited to, “linked nucleotides.” As used herein, “linked nucleosides” are nucleosides that are connected in a continuous sequence (i.e., no additional nucleosides are present between those that are linked).

As used herein, “nucleobase” means a group of atoms that can be linked to a sugar moiety to create a nucleoside that is capable of incorporation into an oligonucleotide, and wherein the group of atoms is capable of bonding, more specifically hydrogen bonding, with a complementary naturally occurring nucleobase of another oligonucleotide or nucleic acid. Nucleobases may be naturally occurring or may be modified.

As used herein the terms, “unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methyl C), and uracil (U).

As used herein, “modified nucleobase,” means any nucleobase that is not a naturally occurring nucleobase.

As used herein, “modified nucleoside” means a nucleoside comprising at least one chemical modification compared to naturally occurring RNA or DNA nucleosides. Modified nucleosides can comprise a modified sugar moiety and/or a modified nucleobase.

As used herein, “bicyclic nucleoside” or “BNA” means a nucleoside comprising a bicyclic sugar moiety.

As used herein, “locked nucleic acid nucleoside” or “LNA” means a nucleoside comprising a bicyclic sugar moiety comprising a 4′-CH2-O-2′bridge.

As used herein, “2′-substituted nucleoside” means a nucleoside comprising a substituent at the 2′-position of the sugar moiety other than H or OH. Unless otherwise indicated, a 2′-substituted nucleoside is not a bicyclic nucleoside.

As used herein, “deoxynucleoside” means a nucleoside comprising 2′-H furanosyl sugar moiety, as found in naturally occurring deoxyribonucleosides (DNA). In certain embodiments, a 2′-deoxynucleoside comprises a modified nucleobase or comprises an RNA nucleobase (e.g., uracil).

As used herein, “oligonucleotide” means a compound comprising a plurality of linked nucleosides. In certain embodiments, an oligonucleotide comprises one or more unmodified ribonucleosides (RNA) and/or unmodified deoxyribonucleosides (DNA) and/or one or more modified nucleosides.

As used herein, “modified oligonucleotide” means an oligonucleotide comprising at least one modified nucleoside and/or at least one modified internucleoside linkage.

Optional modified internucleoside linkages are those, which confer increased stability as compared to the naturally occurring phosphodiesters. “Stability” refers in particular to stability against hydrolysis including enzyme-catalyzed hydrolysis, enzymes including exonucleases and endonucleases.

Optional positions for such modified internucleoside linkages include the termini and the hairpin loop of single-stranded oligomeric compounds of the disclosure. For example, the internucleoside linkages connecting first and second nucleoside and second and third nucleoside counting from the 5′ terminus, and/or the internucleoside linkages connecting first and second nucleoside and second and third nucleoside counting from the 3′ terminus are modified. In addition, a linkage connecting the terminal nucleoside of the 3′ terminus with a ligand, such as GalNAc, may be modified.

As discussed above, optional positions are in the hairpin loop of the single-stranded oligomeric compounds. In particular, all linkages, all but one linkage or the majority of linkages in the hairpin loop are modified. As used herein, “linkages in the hairpin loop” designates the linkages between nucleosides, which are not engaged in base pairing. For example, in a hairpin loop consisting of five nucleosides, there are four linkages between nucleosides which are not engaged in base pairing. Optionally, the term “linkages in the hairpin loop” also extends to the linkages connecting the stem to the loop, i.e., those linkages which connect a base-paired nucleoside to a non-based paired nucleoside. Generally, there are two such positions in hairpins and mxRNAs in accordance with the disclosure.

Most optional is that modified internucleoside linkages are at both termini and in the hairpin loop.

As used herein, “linkage” or “linking group” means a group of atoms that link together two or more other groups of atoms.

As used herein “internucleoside linkage” means a covalent linkage between adjacent nucleosides in an oligonucleotide.

As used herein “naturally occurring internucleoside linkage” means a 3′ to 5′ phosphodiester linkage.

As used herein, “modified internucleoside linkage,” means any internucleoside linkage other than a naturally occurring internucleoside linkage. In particular, a “modified internucleoside linkage” as referred to herein can include a modified phosphorous linking group such as a phosphorothioate or phosphorodithioate internucleoside linkage.

As used herein, “terminal internucleoside linkage” means the linkage between the last two nucleosides of an oligonucleotide or defined region thereof.

As used herein, “phosphorus linking group” means a linking group comprising a phosphorus atom and can include naturally occurring phosphorous linking groups as present in naturally occurring RNA or DNA, such as phosphodiester linking groups, or modified phosphorous linking groups that are not generally present in naturally occurring RNA or DNA, such as phosphorothioate or phosphorodithioate linking groups. Phosphorus linking groups can therefore include without limitation, phosphodiester, phosphorothioate, phosphorodithioate, phosphonate, methylphosphonate, phosphoramidate, phosphorothioamidate, thionoalkylphosphonate, phosphotriesters, thionoalkylphosphotriester and boranophosphate.

As used herein, “internucleoside phosphorus linking group” means a phosphorus linking group that directly links two nucleosides.

As used herein, “oligomeric compound” means a polymeric structure comprising two or more substructures. In certain embodiments, an oligomeric compound comprises an oligonucleotide, such as a modified oligonucleotide. In certain embodiments, an oligomeric compound further comprises one or more conjugate groups and/or terminal groups and/or ligands. In certain embodiments, an oligomeric compound consists of an oligonucleotide. In certain embodiments, an oligomeric compound comprises a backbone of one or more linked monomeric sugar moieties, where each linked monomeric sugar moiety is directly or indirectly attached to a heterocyclic base moiety. In certain embodiments, oligomeric compounds may also include monomeric sugar moieties that are not linked to a heterocyclic base moiety, thereby providing abasic sites. Oligomeric compounds may be defined in terms of a nucleobase sequence only, i.e., by specifying the sequence of A, G, C, U (or T). In such a case, the structure of the sugar-phosphate backbone is not particularly limited and may or may not comprise modified sugars and/or modified phosphates. On the other hand, oligomeric compounds may be more comprehensively defined, i.e., by specifying not only the nucleobase sequence, but also the structure of the backbone, in particular the modification status of the sugars (unmodified, 2′-OMe modified, 2′-F modified etc.) and/or of the phosphates. An mxRNA is one non-limiting example for an oligomeric compound.

As used herein, “nucleic acid construct” or “construct” refers to an assembly of two or more, such as four oligomeric compounds. The oligomeric compounds may be connected to each other by covalent bonds such phosphodiester bonds as they occur in naturally occurring nucleic acids or modified versions thereof as disclosed herein, or by non-covalent bonds such as hydrogen bonds, optionally hydrogen bonds between nucleobases such as Watson-Crick base pairing. In certain embodiments, optional is that a construct comprises four oligomeric compounds, two of which are connected covalently, thereby giving rise to two nucleic acid strands which nucleic acid strands are bound to each other by hydrogen bonds. Complementarity between the strand may be throughout, but is not necessarily so. In particular, exemplary embodiments provide for an antisense strand targeting a first region of C5 mRNA to be connected covalently with a sense strand of another C5-targeting double stranded RNA molecule, and of the antisense strand of the C5 mRNA-targeting double stranded RNA molecule to be connected covalently to a sense strand of the other C5 mRNA-targeting double stranded RNA molecule. Since antisense and sense strands of the parent single-target-directed RNA molecules do not need to have the same length and optionally do not have the same length with antisense portions being longer than sense portions, an optional construct of the disclosure contains a central region where the 3′ regions of the antisense portions of the parent single-target-directed RNA molecules face each other. In that region generally no or only partial base pairing will occur, while full complementarity is not excluded. Otherwise, where antisense and sense portions of the respective parent RNA molecules face each other; there is complementarity, optionally full complementarity or 1 or 2 mismatches. An muRNA is non-limiting example for a nucleic acid construct.

The term “strand” has its art-established meaning and refers to a plurality of linked nucleosides, the linker not being particularly limited, but including phosphodiesters and variants thereof as disclosed herein. A strand may also be viewed as a plurality of linked nucleotides in which case the linker would be a covalent bond.

As used herein, “terminal group” means one or more atom attached to either, or both, the 3′ end or the 5′ end, also called “terminus” of an oligonucleotide. In certain embodiments, a terminal group comprises one or more terminal group nucleosides, whereas a “terminal nucleoside” is only one nucleotide at the respective end (5′ end or 3′ end).

As used herein, “conjugate” or “conjugate group” means an atom or group of atoms bound to an oligonucleotide or oligomeric compound. In certain embodiments, a conjugate group links a ligand to a modified oligonucleotide or oligomeric compound. In general, conjugate groups can modify one or more properties of the compound to which they are attached, including, but not limited to pharmacodynamic, pharmacokinetic, binding, absorption, cellular distribution, cellular uptake, and charge and/or clearance properties.

As used herein, “conjugate linker” or “linker” in the context of a conjugate group means a portion of a conjugate group comprising any atom or group of atoms and which covalently link an oligonucleotide to another portion of the conjugate group. In certain embodiments, the point of attachment on the oligomeric compound is the 3′-oxygen atom of the 3′-hydroxyl group of the 3′ terminal nucleoside of the oligonucleotide. In certain embodiments, the point of attachment on the oligomeric compound is the 5′-oxygen atom of the 5′-hydroxyl group of the 5′ terminal nucleoside of the oligonucleotide. In certain embodiments, the bond for forming attachment to the oligomeric compound is a cleavable bond. In certain such embodiments, such cleavable bond constitutes all or part of a cleavable moiety.

In certain embodiments, conjugate groups comprise a cleavable moiety (e.g., a cleavable bond or cleavable nucleoside) and ligand portion that can comprise one or more ligands, such as a carbohydrate cluster portion, such as an N-Acetyl-Galactosamine, also referred to as “GalNAc”, cluster portion. In certain embodiments, the carbohydrate cluster portion is identified by the number and identity of the ligand. For example, in certain embodiments, the carbohydrate cluster portion comprises 2 GalNAc groups. For example, in certain embodiments, the carbohydrate cluster portion comprises 3 GalNAc groups and this is particularly optional. In certain embodiments, the carbohydrate cluster portion comprises 4 GalNAc groups. Such ligand portions are attached to an oligomeric compound via a cleavable moiety, such as a cleavable bond or cleavable nucleoside. The ligands can be arranged in a linear or branched configuration, such as a biantennary or triantennary configurations. An optional carbohydrate cluster has the following formula:

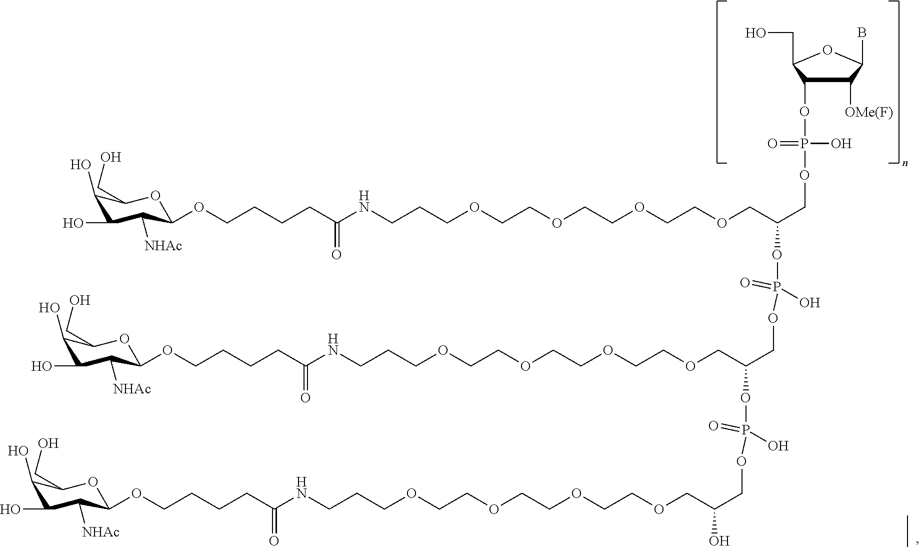

wherein in the structural formula one, two, or three phosphodiester linkages can also be substituted by phosphorothioate linkages.

As used herein, “cleavable moiety” means a bond or group that is capable of being cleaved under physiological conditions. In certain embodiments, a cleavable moiety is cleaved inside a cell or sub-cellular compartments, such as an endosome or lysosome. In certain embodiments, a cleavable moiety is cleaved by endogenous enzymes, such as nucleases. In certain embodiments, a cleavable moiety comprises a group of atoms having one, two, three, four, or more than four cleavable bonds. In certain embodiments, a cleavable moiety is a phosphodiester linkage.

As used herein, “cleavable bond” means any chemical bond capable of being broken.

As used herein, “carbohydrate cluster” means a compound having one or more carbohydrate residues attached to a linker group.

As used herein, “modified carbohydrate” means any carbohydrate having one or more chemical modifications relative to naturally occurring carbohydrates.

As used herein, “carbohydrate derivative” means any compound which may be synthesized using a carbohydrate as a starting material or intermediate.

As used herein, “carbohydrate” means a naturally occurring carbohydrate, a modified carbohydrate, or a carbohydrate derivative. A carbohydrate is a biomolecule including carbon (C), hydrogen (H) and oxygen (O) atoms. Carbohydrates can include monosaccharide, disaccharides, trisaccharides, tetrasaccharides, oligosaccharides or polysaccharides, such as one or more galactose moieties, one or more lactose moieties, one or more N-Acetyl-Galactosamine moieties, and/or one or more mannose moieties. A particularly optional carbohydrate is N-Acetyl-Galactosamine.

As used herein, “strand” means an oligomeric compound comprising linked nucleosides.

As used herein, “single strand” or “single-stranded” means an oligomeric compound comprising linked nucleosides that are connected in a continuous sequence without a break there between. Such single strands may include regions of sufficient self-complementarity so as to be capable of forming a stable self-duplex in a hairpin structure.

As used herein, “hairpin” means a single stranded oligomeric compound that includes a duplex formed by base pairing between sequences in the strand that are self-complementary and opposite in directionality.

As used herein, “hairpin loop” means an unpaired loop of linked nucleosides in a hairpin that is created as a result of hybridization of the self-complementary sequences. The resulting structure looks like a loop or a U-shape.

In particular, short hairpin RNA, also denoted as shRNA, comprises a duplex region and a loop connecting the regions forming the duplex. The end of the duplex region, which does not carry the loop, may be blunt-ended or carry (a) 3′ and/or (a) 5′ overhang(s). Optional are blunt-ended constructs. The term “shRNA” is more generic than “mxRNA”, as defined below, and may include compounds in which the loop is not or not exclusively formed out of an antisense strand. In particular, shRNA includes an antisense strand, also called guide strand, being complementary to a region of a target RNA, and a sense strand, i.e., a passenger strand, being substantially complementary to the antisense strand. More particularly, the antisense strand and the sense strand within the shRNA are directly linked, e.g., by a phosphate or a phosphorothioate, or linked by a third portion of linked nucleosides forming the loop, which means that the 3′ end of the antisense strand is linked to the 5′ end of the sense strand via covalent bonding over several other groups. Such direct linkage does not include a gap or nick.

As used herein, “directionality” means the end-to-end chemical orientation of an oligonucleotide based on the chemical convention of numbering of carbon atoms in the sugar moiety meaning that there will be a 5′-end defined by the 5′ carbon of the sugar moiety, and a 3′-end defined by the 3′ carbon of the sugar moiety. In a duplex or double stranded oligonucleotide, the respective strands run in opposite 5′ to 3′ directions to permit base pairing between them.

As used herein, “duplex”, or also abbreviated as “dup”, means two or more complementary strand regions, or strands, of an oligonucleotide or oligonucleotides, hybridized together by way of non-covalent, sequence-specific interaction there between. Most commonly, the hybridization in the duplex will be between nucleobases adenine (A) and thymine (T), and/or (A) adenine and uracil (U), and/or guanine (G) and cytosine (C). The duplex may be part of a single stranded structure, wherein self-complementarity leads to hybridization, or as a result of hybridization between respective strands in a double stranded construct.

As used herein, “double strand” or “double stranded” means a pair of oligomeric compounds that are hybridized to one another. In certain embodiments, a double-stranded oligomeric compound comprises a first and a second oligomeric compound.

As used herein, “expression” means the process by which a gene ultimately results in a protein. Expression includes, but is not limited to, transcription, post-transcriptional modification (e.g., splicing, polyadenylation, addition of 5′-cap), and translation.

As used herein, “transcription” or “transcribed” refers to the first of several steps of DNA based gene expression in which a target sequence of DNA is copied into RNA (especially mRNA) by the enzyme RNA polymerase. During transcription, a DNA sequence is read by an RNA polymerase, which produces a complementary, antiparallel RNA sequence called a primary transcript.

As used herein, “target sequence” means a sequence to which an oligomeric compound is intended to hybridize to result in a desired activity with respect to C5 expression. Oligonucleotides have sufficient complementarity to their target sequences to allow hybridization under physiological conditions.

As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In both DNA and RNA, guanine (G) is complementary to cytosine (C). In certain embodiments, complementary nucleobase means a nucleobase of an oligomeric compound that is capable of base pairing with a nucleobase of its target sequence. For example, if a nucleobase at a certain position of an oligomeric compound is capable of hydrogen bonding with a nucleobase at a certain position of a target sequence, then the position of hydrogen bonding between the oligomeric compound and the target sequence is considered complementary at that nucleobase pair. Nucleobases comprising certain modifications may maintain the ability to pair with a counterpart nucleobase and thus, are still capable of nucleobase complementarity.

As used herein, “non-complementary” in reference to nucleobases means a pair of nucleobases that do not form hydrogen bonds with one another.

As used herein, “complementary” in reference to oligomeric compounds (e.g., linked nucleosides, oligonucleotides) means the capacity of such oligomeric compounds or regions thereof to hybridize to a target sequence, or to a region of the oligomeric compound itself, through nucleobase complementarity.

Complementary oligomeric compounds need not have nucleobase complementarity at each nucleoside. Rather, some mismatches are tolerated. In certain embodiments, complementary oligomeric compounds or regions are complementary at 70% of the nucleobases (70% complementary). In certain embodiments, complementary oligomeric compounds or regions are 80%>complementary. In certain embodiments, complementary oligomeric compounds or regions are 90%> complementary. In certain embodiments, complementary oligomeric compounds or regions are at least 95% complementary. In certain embodiments, complementary oligomeric compounds or regions are 100% complementary.

As used herein, “self-complementarity” in reference to oligomeric compounds means a compound that may fold back on itself, creating a duplex as a result of nucleobase hybridization of internal complementary strand regions. Depending on how close together and/or how long the strand regions are, then the compound may form hairpin loops, junctions, bulges or internal loops.

As used herein, “mismatch” means a nucleobase of an oligomeric compound that is not capable of pairing with a nucleobase at a corresponding position of a target sequence, or at a corresponding position of the oligomeric compound itself when the oligomeric compound hybridizes as a result of self-complementarity, when the oligomeric compound and the target sequence and/or self-complementary regions of the oligomeric compound, are aligned.

As used herein, “hybridization” means the pairing of complementary oligomeric compounds (e.g., an oligomeric compound and its target sequence). While not limited to a particular mechanism, the most common mechanism of pairing involves hydrogen bonding, which may be Watson-Crick, Hoogsteen or reversed Hoogsteen hydrogen bonding, between complementary nucleobases.

As used herein, “specifically hybridizes” means the ability of an oligomeric compound to hybridize to one nucleic acid site with greater affinity than it hybridizes to another nucleic acid site.

As used herein, “fully complementary” in reference to an oligomeric compound or region thereof means that each nucleobase of the oligomeric compound or region thereof is capable of pairing with a nucleobase of a complementary nucleic acid target sequence or a self-complementary region of the oligomeric compound. Thus, a fully complementary oligomeric compound or region thereof comprises no mismatches or unhybridized nucleobases with respect to its target sequence or a self-complementary region of the oligomeric compound.

As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

As used herein, “percent identity” means the number of nucleobases in a first nucleic acid that are the same type (independent of chemical modification) as nucleobases at corresponding positions in a second nucleic acid, divided by the total number of nucleobases in the first nucleic acid.

As used herein, “modulation” means a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression.

As used herein, “type of modification” in reference to a nucleoside or a nucleoside of a “type” means the chemical modification of a nucleoside and includes modified and unmodified nucleosides. Accordingly, unless otherwise indicated, a “nucleoside having a modification of a first type” may be an unmodified nucleoside.

As used herein, “differently modified” mean chemical modifications or chemical substituents that are different from one another, including absence of modifications. Thus, for example, an MOE nucleoside and an unmodified naturally occurring RNA nucleoside are “differently modified,” even though the naturally occurring nucleoside is unmodified. Likewise, DNA and RNA oligonucleotides are “differently modified,” even though both are naturally occurring unmodified nucleosides. Nucleosides that are the same but for comprising different nucleobases are not differently modified. For example, a nucleoside comprising a 2′-OMe modified sugar moiety and an unmodified adenine nucleobase and a nucleoside comprising a 2′-OMe modified sugar moiety and an unmodified thymine nucleobase are not differently modified.

As used herein, “the same type of modifications” refers to modifications that are the same as one another, including absence of modifications. Thus, for example, two unmodified RNA nucleosides have “the same type of modification,” even though the RNA nucleosides are unmodified. Such nucleosides having the same type of modification comprises different nucleobases.

As used herein, “region” or “regions”, or “portion” or “portions”, mean a plurality of linked nucleosides that have a function or character as defined herein, in particular with reference to the claims and definitions as provided herein. Typically, such regions or portions comprise at least 10, at least 11, at least 12 or at least 13 linked nucleosides. For example, such regions comprise 13 to 20 linked nucleosides, such as 13 to 16 or 18 to 20 linked nucleosides. Typically, a first region as defined herein comprises 18 to 20 nucleosides and a second region as defined herein comprises 13 to 16 linked nucleosides.

As used herein, “pharmaceutically acceptable carrier or diluent” means any substance suitable for use in administering to an animal. In certain embodiments, a pharmaceutically acceptable carrier or diluent is sterile saline. In certain embodiments, such sterile saline is pharmaceutical grade saline.

As used herein, “substituent” and “substituent group,” means an atom or group that replaces the atom or group of a named parent compound. For example, a substituent of a modified nucleoside is any atom or group that differs from the atom or group found in a naturally occurring nucleoside (e.g., a modified 2′-substituent is any atom or group at the 2′-position of a nucleoside other than H or OH). Substituent groups can be protected or unprotected. In certain embodiments, compounds of the present disclosure have substituents at one or at more than one position of the parent compound. Substituents may also be further substituted with other substituent groups and may be attached directly or via a linking group such as oxygen or an alkyl or hydrocarbyl group to a parent compound.

Such substituents can be present as the modification on the sugar moiety, in particular a substituent present at the 2′-position of the sugar moiety. Unless otherwise indicated, groups amenable for use as substituents include without limitation, one or more of halo, hydroxyl, alkyl, alkenyl, alkynyl, acyl, carboxyl, alkoxy, alkoxyalkylene and amino substituents. Certain substituents as described herein can represent modifications directly attached to a ring of a sugar moiety (such as a halo, such as fluoro, directly attached to a sugar ring), or a modification indirectly linked to a ring of a sugar moiety by way of an oxygen linking atom that itself is directly linked to the sugar moiety (such as an alkoxyalkylene, such as methoxyethylene, linked to an oxygen atom, overall providing an MOE substituent as described herein attached to the 2′-position of the sugar moiety).

As used herein, “alkyl,” as used herein, means a saturated straight or branched monovalent C1-6 hydrocarbon radical, with methyl being a most optional alkyl as a substituent at the 2′-position of the sugar moiety. The alkyl group typically attaches to an oxygen linking atom at the 2′position of the sugar, therefore, overall providing a —Oalkyl substituent, such as an —OCH3 substituent, on a sugar moiety of an oligomeric compound according to the present disclosure. This will be well understood be a person skilled in the art.

As used herein, “alkylene” means a saturated straight or branched divalent hydrocarbon radical of the general formula —CnH2n- where n is 1-6. Methylene or ethylene are optional alkylenes.

As used herein, “alkenyl” means a straight or branched unsaturated monovalent C2-6 hydrocarbon radical, with ethenyl or propenyl being most optional alkenyls as a substituent at the 2′-position of the sugar moiety. As will be well understood in the art, the degree of unsaturation that is present in an alkenyl radical is the presence of at least one carbon to carbon double bond. The alkenyl group typically attaches to an oxygen linking atom at the 2′-position of the sugar, therefore, overall providing a —Oalkenyl substituent, such as an —OCH2CH═CH2 substituent, on a sugar moiety of an oligomeric compound according to the present disclosure. This will be well understood be a person skilled in the art.

As used herein, “alkynyl” means a straight or branched unsaturated C2-6 hydrocarbon radical, with ethynyl being a most optional alkynyl as a substituent at the 2′-position of the sugar moiety. As will be well understood in the art, the degree of unsaturation that is present in an alkynyl radical is the presence of at least one carbon to carbon triple bond. The alkynyl group typically attaches to an oxygen linking atom at the 2′-position of the sugar, therefore, overall providing a —Oalkynyl substituent on a sugar moiety of an oligomeric compound according to the present disclosure. This will be well understood be a person skilled in the art.

As used herein, “carboxyl” is a radical having a general formula —CO2H.

As used herein, “acyl” means a radical formed by removal of a hydroxyl group from a carboxyl radical as defined herein and has the general Formula —C(O)—X where X is typically C1-6 alkyl.

As used herein, “alkoxy” means a radical formed between an alkyl group, such as a C1-6 alkyl group, and an oxygen atom wherein the oxygen atom is used to attach the alkoxy group either to a parent molecule (such as at the 2′-position of a sugar moiety), or to another group such as an alkylene group as defined herein. Examples of alkoxy groups include without limitation, methoxy, ethoxy, propoxy, isopropoxy, n-butoxy, sec-butoxy and tert-butoxy. Alkoxy groups as used herein may optionally include further substituent groups.

As used herein, alkoxyalkylene means an alkoxy group as defined herein that is attached to an alkylene group also as defined herein, and wherein the oxygen atom of the alkoxy group attaches to the alkylene group and the alkylene attaches to a parent molecule. The alkylene group typically attaches to an oxygen linking atom at the 2′-position of the sugar, therefore, overall providing a —Oalkylenealkoxy substituent, such as an —OCH2CH2OCH3 substituent, on a sugar moiety of an oligomeric compound according to the present disclosure. This will be well understood by a person skilled in the art and is generally referred to as an MOE substituent as defined herein and as known in the art.

As used herein, “amino” includes primary, secondary and tertiary amino groups.

As used herein, “halo” and “halogen,” mean an atom selected from fluorine, chlorine, bromine and iodine.

As used herein, the term “mxRNA” is in particular understood as defined in WO 2020/044186 A2, which is incorporated by reference herein in its entirety. In particular, an mxRNA is a hairpin-shaped RNA molecule consisting of an antisense portion (also referred to as the guide strand) and a sense portion (also referred to the passenger strand). The mxRNA comprises duplex region and a hairpin loop, wherein the mxRNA has an approximate length of about 34 nucleotides. The duplex region comprises a region in which parts of the antisense portion and substantially the entire sense portion, typically 14 or 15 nucleotides of each strand, are base-paired. The hairpin loop connects both regions, i.e., antisense region and sense region, of that duplex via e.g. a phosphate or a phosphorothioate linker, i.e. covalently, while the antisense portion typically has a length of about 18 to 20 nucleotides and, therefore, forms the antisense duplex region and the loop. The loop, of which the antisense portion is part, furthermore connects the sense, forming the second strand of the loop, and the antisense portion.

The term “angiotensinogen” or abbreviated “AGT”, also known as SERPINA 8 or ANHU, is used in its common sense and denotes a protein produced in the liver which is a component of the renin-angiotensin-aldosterone-system (RAAS), and which is converted to angiotensin I by renin when released in circulation. Angiotensinogen is expressed and produced in the liver by the angiotensin gene or “AGT gene”.

As used herein, the term “muRNA” or “multi RNA” includes nucleic acid constructs comprising more than one, typically two, RNA sequences, i.e., first and second nucleic acid portions, targeting different regions of C5 mRNA; or one region of C5 mRNA and an mRNA region of another target molecule. The targeting RNA sequences are also referred to as “antisense” or “guide” strands, while the respective passenger strands, i.e., third and fourth nucleic acid portions being complementary to the first and second portion, respectively, are also included in the nucleic acid construct. In particular, such muRNA are designed such that subsequent to in vivo administration, they are disassembled and the first and second nucleic acid portions are released. A particular example for such muRNA is shown below, where (1) is the first nucleic acid portion, (2) is the third nucleic acid portion being complementary to (1), (3) is the second nucleic acid portion being complementary to the fourth nucleic acid portion, while (5) is a labile linker while (6) is a ligand, which will both be explained below.

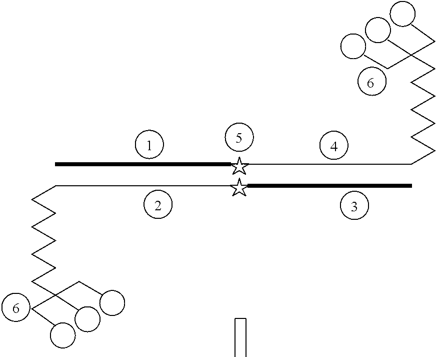

It will also be understood that oligomeric compounds as described herein may have one or more non-hybridizing nucleosides at one or both ends of one or both strands (overhangs) and/or one or more internal non-hybridizing nucleosides (mismatches) provided there is sufficient complementarity to maintain hybridization under physiologically relevant conditions. Alternatively, oligomeric compounds as described herein may be blunt ended at least one end.

As used herein, the term “complement component C5” or just “C5” denotes the corresponding and commonly known protein, which decomposes into C5a and C5b, wherein C5b forms part of the membrane attack complex at the late stage of the complement activation. C5 is a protein that is in humans encoded by the C5 gene. Complement component C5 is the fifth component of complement, which plays an important role in inflammatory and cell killing processes. This protein is composed of alpha and beta polypeptide chains that are linked by a disulfide bridge. An activation peptide, C5a, which is an anaphylatoxin that possesses potent spasmogenic and chemotactic activity, is derived from the alpha polypeptide via cleavage with a C5-convertase. The C5b macromolecular cleavage product can form a complex with the C6 complement component, and this complex is the basis for formation of the membrane attack complex, which includes additional complement components.

The term “comprising” is used herein to mean including the method steps or elements identified, but that such steps or elements do not comprise an exclusive list and as such, there may be present additional steps or elements.

Further, to the extent that the term “includes” is used in either the detailed description or the claims, such term is intended to be inclusive in a manner similar to the term “comprising” as “comprising” is interpreted when employed as a transitional word in a claim.

### The Present Disclosure Relates to the Following Aspects and Embodiments

Small Hairpin (shRNA) and mxRNA Oligomeric Compounds

According to a first aspect, the present disclosure is directed to an oligomeric compound capable of inhibiting expression of complement factor C5 (C5), wherein the compound comprises at least a first region of linked nucleosides having at least a first nucleobase sequence that is at least partially complementary to at least a portion of RNA transcribed from an C5 gene, wherein the first nucleobase sequence is selected from the following sequences, or a portion thereof: sequences of Table 1a (SEQ ID NOs: 1 to 250), wherein the portion optionally has a length of at least 18 nucleosides. In particular, the 5′ terminal nucleoside of the first nucleobase sequence can contain U instead of A; or U instead of G; or U instead of C, respectively.

In certain embodiments, the oligomeric compound further comprises at least a second region of linked nucleosides having at least a second nucleobase sequence that is at least partially complementary to the first nucleobase sequence and is selected from the following sequences, or a portion thereof: sequences of Table 1b (SEQ ID NOs: 251 to 500), wherein the portion optionally has a length of at least 8, 9, 10 or 11, more optionally at least 10, nucleosides.

In particular, the 3′ terminal nucleoside of the second nucleobase sequence may contain an A instead of U, G or C, respectively; and more particularly the nucleobase A as a complementary nucleobase to the 5′ terminal nucleoside of the first nucleobase sequence.

The first region of linked nucleosides is also referred to as antisense region or guide region/strand, and the second region of linked nucleosides is referred to as sense region or passenger region/strand. As disclosed in optional embodiments below, the two regions may be located on the same RNA strand, optionally in an adjacent manner. This gives rise to hairpin molecules, also referred to as mxRNAs. On the other hand, the two regions may be located on separate strands, which gives rise to double-stranded RNAs (dsRNAs), wherein optionally each strand comprises the respective region.

Without wishing to be bound by theory, it is assumed that the inventive oligomeric compounds set forth above including the first region of linked nucleosides and the second region of linked nucleosides are, during the process of RNA interference, capable of being cleaved by the protein Argonaute 2 (Ago2). The mxRNA is incorporated into the RNA-induced silencing complex (RISC). The RISC assembly then binds and degrades the target mRNA. Specifically, this is accomplished when the guide strand pairs with a complementary sequence in an C5 mRNA molecule and induces cleavage by Ago2, a catalytic component of the RISC. For that reason, as the expression of C5 is inhibited, it is believed effects correlating with overregulation of angiotensin II by the pathway described above, is inhibited too.

In certain embodiments the first nucleobase sequence is selected from the following sequences, or a portion thereof: SEQ ID NOs: 61, 30, 37, 87, 55, 66, 23, 83, 43, 47, 72, 27, 14, 28, 46, 82, 74, 75, 73, 53, 16, 36, 59, 42, and 56. Optionally the first nucleobase sequence is selected from the following sequences, or a portion thereof: SEQ ID NOs: 61, 30, 37, 83 82, 74, 75, 73, 53, 16, 36, 59, 42, and 56, optionally 30, 37, and 83, more optionally 30 and 37, most optionally 30.

In an optional embodiment thereof, the second nucleobase sequence is selected from the following sequences, or a portion thereof: SEQ ID NOs: 311, 280, 287, 333, 332, 324, 325, 323, 303, 266, 286, 309, 292, 306 and, optionally 280, 287, and 333, more optionally 30 and 37, most optionally 30.

Therefore, the oligomeric compound according to the present disclosure can comprise SEQ ID NOs 30+280 or 37+287.

### Lengths and Molecular Features of the Oligomeric Compounds According to the First Aspect

The first region of linked nucleosides comprise 18 to 35, optionally 18 to 20, more optionally 18 or 19, and yet more optionally 19 linked nucleosides. In addition, the second region of linked nucleosides comprises 10 to 35, optionally 10 to 20, more optionally 10 to 16, and yet more optionally 10 to 15, in particular 13, 14 or 15 linked nucleosides.

The oligomeric compound including the first and second regions of linked nucleosides comprises at least one complementary duplex region that comprises at least a portion of the first region of linked nucleosides directly or indirectly linked to at least a portion of the second region of linked nucleosides, wherein optionally the duplex region has a length of 10 to 19, more optionally 12 to 19, and yet more optionally 12 to 15, in particular 14 or 15, base pairs, wherein optionally there is one mismatch within the duplex region.

In certain embodiments, each of the first and second regions of linked nucleosides has a 5′ to 3′ directionality thereby defining 5′ and 3′ regions respectively thereof.

In the oligomeric compound having the first and second regions of linked nucleosides having a 5′ to 3′ directionality, the 5′ region of the first region of linked nucleosides may be directly or indirectly linked to the 3′ region of the second region of linked nucleosides, for example by complementary base pairing, wherein optionally the 5′ terminal nucleoside of the first nucleoside region base pairs with the 3′ terminal nucleoside of the second nucleoside region.

In the aforementioned embodiments, the 3′ region of the first region of linked nucleosides may be directly or indirectly linked to the 5′ region of the second region of linked nucleosides, wherein optionally the first nucleoside region is directly and covalently linked to the second nucleoside region such as by a phosphate, a phosphorothioate, or a phosphorodithioate, wherein more optionally a 3′ terminal nucleoside of the first region of linked nucleosides is directly and covalently linked to a 5′ terminal nucleoside of the second region of linked nucleosides by a phosphate, a phosphorothioate, or a phosphorodithioate. It is particularly optional that the 3′ terminal nucleoside of the first region is directly linked to the 5′ terminal nucleoside of the second region via a phosphorothioate internucleoside linkage.

This amounts to the formation of a single oligonucleotide comprising two regions being directly fused to each other. Owing to the base pairing as defined in the previous embodiment, such oligonucleotide will assume a hairpin configuration. Optimized hairpins, especially in terms of size, are the subject of further embodiments below.

In certain embodiments, the oligomeric compound comprises the first region of linked nucleosides and the second region of linked nucleosides.

Each of the regions may constitute a separate strand, thereby giving rise to a double-stranded RNA (dsRNA). Particularly optional dsRNAs of the disclosure are those with a length of the first strand of 19 nucleosides and a length of the second region of 14 or 15, optionally 14 nucleosides. When used for defining the length of a region or strand, the terms “nucleoside” and “nucleotide” (sometimes abbreviated “nt”) are used equivalently.

In the alternative, and as stated above, the two regions may be fused together, giving rise to a hairpin.

In certain embodiments, there may be an intervening third region of linked nucleosides between the first and the second region.

In optional embodiments, the oligomeric compound comprises or consists of a single strand comprising or consisting of the first, the third, and the second nucleoside regions, wherein at least a portion of the first nucleoside region is directly or indirectly linked to at least a portion of the second nucleoside region so as to form the at least partially complementary duplex region.

In other words, the oligomeric compound comprises a single strand comprising the first and second nucleoside regions, wherein at least a portion of the first nucleoside region is directly or indirectly linked to at least a portion of the second nucleoside region so as to form the at least partially complementary duplex region. As noted above, the third region is optional.

In certain embodiments, the oligomeric compound comprises consists of a single strand comprising or consisting of the first and second regions of linked nucleosides, wherein at least a portion of the first region of linked nucleosides is directly or indirectly linked to at least a portion of the second region of linked nucleosides so as to form the at least partially complementary duplex region.

In the oligomeric compound, which comprises or consists of a single strand, the first and the second nucleoside regions are directly adjacent on the single strand.

In certain embodiments, the first nucleoside region may have a greater number of linked nucleosides compared to the second nucleoside region.

Optionally, a ratio between a total number of linked nucleosides of the first nucleoside region and a total number of linked nucleosides of the second nucleoside region ranges from about 19/15 to about 19/8 or from about 18/15 to about 18/8. In particularly optional embodiments, the ratio is 19/15, 19/14, 19/13, 18/15, 18/14 or 18/13, most optionally 19/14 or

Alternatively, or in addition, a percentage of the total number of linked nucleosides of the first nucleoside region relative to the total number of nucleosides of the oligomeric compound may range from about to about 55% to about 60%. In particularly optional embodiments, the percentage may range from 57% to about 59.5%, most optionally the percentage is about 57.6% or about 59.4%.

Without wishing to be bound by theory, it is assumed that the ratio and/or percentages as mentioned above provides a suitable ratio/percentage of the number of nucleotides in the antisense (guide) strand and the number of nucleotides in the sense (passenger) strand to be processed by the RISC complex as mentioned above without being significantly degraded before, and therefore, for being effective in C5 knockdown.

In the oligomeric compound having a greater number of linked nucleotides in the first region than in the second region, the additional number of linked nucleosides of the first nucleoside region form a hairpin loop linking the first and second regions of linked nucleosides, wherein optionally a part of the first nucleobase sequence of the first nucleobase sequence being complementary RNA transcribed from an C5 gene forms the hairpin loop, wherein the loop comprises 2 to 5, optionally 4 or 5, nucleosides.

Such compounds are also referred to as hairpins or mxRNAs herein. Owing to the second region being shorter as compared to the first region, the compound is optimized in terms of size (or miniaturized) as compared to a conventional siRNA, which has two regions of comparable length.

Optionally, the loop has 4 or 5 linked nucleosides. Particularly optional is a length of the first region of 19 nucleosides, of the second region of 14 nucleosides, and of the hairpin loop of 5 nucleosides, wherein the 5 nucleosides in the hairpin are the 5 3′-terminal nucleosides of the first region. Such molecular architecture of a hairpin or mxRNA of the disclosure is also designated “14-5-14” herein.

In certain embodiments, an oligomeric single strand as disclosed earlier herein, can be selected from Table 2, in particular SEQ ID NOs: 561, 530, 537, 587, 555, 566, 523, 583, 543, 547, 572, 527, 514, 528, 546, 582, 574, 575, 573, 553, 516, 536, 559, 542, and 556, optionally 583, 530, and 537, more optionally 530 and 537, most optionally 530, wherein optionally the 5′ terminal nucleoside of the first region of linked nucleosides includes an U as the nucleobase, and the 5′ terminal nucleoside of the second region of linked nucleosides includes an A as the nucleobase.

In particular embodiments, the single strand the single strand is selected from Table 3c, in particular from SEQ ID NOs: 1011, 980, 987, 1037, 1005, 1016, 973, 1033, 993, 997, 1022, 977, 964, 978, 996, 1032, 1024, 1025, 1023, 1003, 966, 986, 1009, 992, 1006, optionally 980, 987, and 1033, more optionally 980 and 987, most optionally 980, wherein optionally the 5′ terminal nucleoside of the first region of linked nucleosides includes an U as the nucleobase, and the 5′ terminal nucleoside of the second region of linked nucleosides includes an A as the nucleobase.

In certain embodiments a hairpin loop as described earlier herein may be present at the 3′ region of the first region of linked nucleosides, wherein optionally one, two or more 3′ terminal nucleosides of the first nucleobase sequence, to the extent the nucleobases of the one, two or more 3′ terminal nucleosides permit, fold back and form or contribute to the second region of linked nucleoside.

This is a structural design also referred to as “spill-over”. It is only possible in those cases where there is self-complementarity between the nucleobases at the 3′-terminal end of the region of the guide sequence comprised in the duplex and the very 3′-terminal nucleobases of the same guide sequence. For example, this could be implemented as a 13-5-13 design, thereby allowing for further miniaturization. The first “13” refers to the region of the guide sequence involved in the duplex, 5 is the length of the loop which is also formed by the guide sequence, and the second 13 refers to the second region of the duplex and is formed by one nucleobase of the guide sequence and 12 nucleobases of the passenger region in 5′ to 3′ direction. As such, a length of the guide sequence of 19 nucleosides is maintained, but the passenger sequence is shortened to 12 nucleosides.

In certain embodiments, in case a third nucleoside region as described earlier herein, the third nucleoside region and optionally a 3′-terminal portion, optionally consisting of 1, 2, 3, 4, 5, 6, 7, 8, 9, or 10 linked nucleosides, of the first nucleoside region and/or a 5′-terminal portion, optionally consisting of 1, 2, 3, 4, 5, 6, 7, 8, 9, or 10 linked nucleosides, of the second nucleoside region may form a hairpin loop.

In certain embodiments wherein the hairpin loop comprises 1 to 8, 2 to 7, 3 to 6, optionally 4 or 5 linked nucleosides.

The oligomeric compounds according to the first aspect disclosed herein may be blunt ended.

In the oligomeric compounds according to the first aspect disclosed herein, either the first or second nucleoside region may have an overhang.

In the oligomeric compounds according to the first aspect disclosed herein, the first region may be selected from the sequences of Table 3a, or a portion thereof, in particular from SEQ ID NOs: 327, 352, 356, 362, 375 and 393.

In the oligomeric compounds according to the first aspect disclosed herein the second region may be selected from the sequences of Table 3b, or a portion thereof, especially a portion having a length of 14 nucleosides, in particular from SEQ ID NOs: 427, 452, 456, 462, 475 and 493.

The oligomeric compound may have a total length of about 25 to about 35 nucleosides, in particular about 33 or about 34 nucleosides.

In certain embodiments, a terminal nucleoside at a 5′ position of the first region has a nucleobase selected from the group consisting of A, U, G and C, optionally U, and, wherein optionally, a terminal nucleoside at a 3′ position of the second region has a base being complementary to the base at the 5′ position of the first region, optionally A.

### Ligands

The oligomeric compounds comprise one or more ligands.

The one or more ligands, in particular two or more or three ligands, may be conjugated to the second region of linked nucleosides and/or the first region of linked nucleosides.

The one or more ligands may be conjugated at the 3′ region, optionally at the 3′ terminal nucleoside of the second region of linked nucleosides and/or of the first region of linked nucleosides, and/or to the 5′ terminal nucleoside of the second region of linked nucleosides. In particular, the ligands may be conjugated to the 3′ terminal nucleoside.

The one or more ligands are any cell directing moiety, such as lipids, carbohydrates, aptamers, vitamins and/or peptides that bind cellular membrane or a specific target on cellular surface.

The one or more ligands comprises one or more, in particular three, carbohydrates.

The one or more, in particular three, carbohydrates can be a monosaccharide, disaccharide, trisaccharide, tetrasaccharide, oligosaccharide or polysaccharide.

The one or more carbohydrates comprises or consists of one or more, in particular three, hexose moieties.

The one or more, in particular three, hexose moieties are one or more galactose moieties, one or more lactose moieties, one or more, in particular three, N-Acetyl-Galactosamine moieties, and/or one or more mannose moieties.

The one or more carbohydrates comprises one or more, in particular three, N-Acetyl-Galactosamine moieties.

Alternatively, the one or more carbohydrates comprises two or more N-Acetyl-Galactosamine moieties, optionally three.

The one or more ligands are attached to the oligomeric compound, optionally to the second region of linked nucleosides thereof, in a linear configuration, or in a branched configuration.

A particularly optional ligand is the following, also referred to as “toothbrush”:

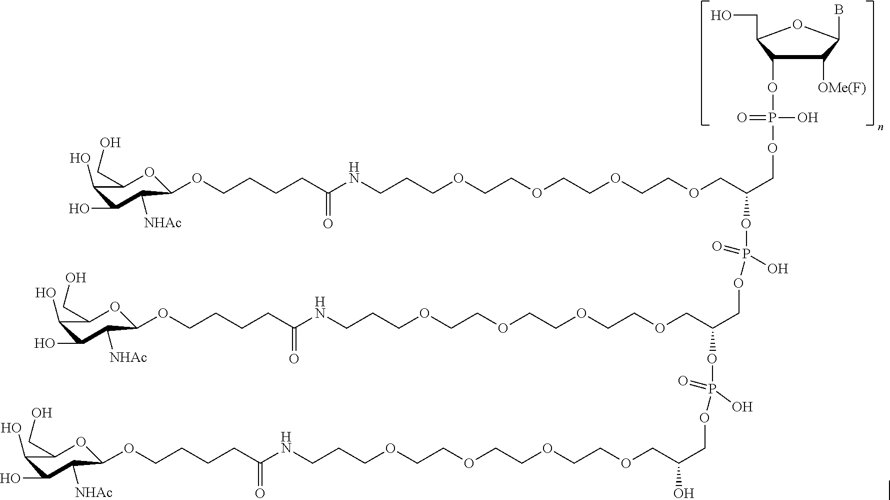

Without wishing to be bound by a particular theory, it is assumed that due to such ligand the target tissue, i.e., the liver where C5 is produced, can be selectively targeted so the oligomeric compounds can exhibit their inhibition of C5 gene more efficiently.

The one or more, in particular three, ligands may be attached to the oligomeric compound as a biantennary or triantennary configuration.

The one or more ligands as discussed above are optionally attached to the 3′ terminal nucleoside of the second region of linked nucleosides.

### Internucleoside Linkages

The oligomeric compound according to the first aspect disclosed herein comprises internucleoside linkages and wherein at least one internucleoside linkage is a modified internucleoside linkage.

The modified internucleoside linkage may be a phosphorothioate or phosphorodithioate internucleoside linkage.

The oligomeric compound according to the first aspect disclosed herein comprises 1 to 16 phosphorothioate or phosphorodithioate internucleoside linkages.

Optional modified internucleoside linkages are subject of the optional embodiments, which follow. Certain modified internucleoside linkages are known in the art and described in, for example, Hu et al., Signal Transduction and Targeted Therapy (2020) 5:101.

The oligomeric compound comprises 7, 8, 9 or 10 phosphorothioate or phosphorodithioate internucleoside linkages. The one or more phosphorothioate or phosphorodithioate internucleoside linkages may present at the 5′ region of the first region of linked nucleosides, wherein optionally, the oligomeric compound comprises three phosphorothioate internucleoside linkages at three adjacent nucleosides at the 5′ region.

In addition, the oligomeric compound comprises phosphorothioate or phosphorodithioate internucleoside linkages between at least two, optionally at least three, optionally at least four, optionally at least five, adjacent nucleosides of the hairpin loop, dependent on the number of nucleosides present in the hairpin loop. Particularly, the oligomeric compound comprises a phosphorothioate or phosphorodithioate internucleoside linkage between each adjacent nucleoside that is present in the hairpin loop.

### Modifications

In the oligomeric compound according to the first aspect of the present disclosure, at least one nucleoside comprises a modified sugar.

The modified sugar may be selected from 2′ modified sugars, a conformationally restricted nucleoside (CRN) sugar such as locked nucleic acid (LNA) sugar, (S)-constrained ethyl bicyclic nucleic acid, and constrained ethyl (cEt) sugar, tricyclo-DNA, morpholino, unlocked nucleic acid (UNA) sugar, glycol nucleic acid (GNA), D-hexitol nucleic acid (HNA), and cyclohexene nucleic acid (CeNA).

Optional modified sugars are subject of the optional embodiments, which follow. Certain modified sugars are known in the art and described in, for example, Hu et al., Signal Transduction and Targeted Therapy (2020) 5:101.

The 2′ modified sugar may be selected from 2′-O-alkyl modified sugar, 2′-O-methyl modified sugar, 2′-O-methoxyethyl modified sugar, 2′-O-allyl modified sugar, 2′-C-allyl modified sugar, 2′-deoxy modified sugar such as 2′-deoxy ribose, 2′-F modified sugar, 2′-arabino-fluoro modified sugar, 2′-O-benzyl modified sugar, and 2′-O-methyl-4-pyridine modified sugar. At least one modified sugar may be a 2′-O-methyl modified sugar.

At least one modified sugar may be a 2′-F modified sugar and, optionally, at most 16 or 17 sugars are 2′-F modified sugars. Optionally, the the sugar is ribose.

In the oligomeric compound according to the first aspect disclosed herein, sugars of the nucleosides at any of positions 2 and 14 downstream from the first nucleoside of the 5′ region of the first region of linked nucleosides, do not contain 2′-O-methyl modifications.

In certain embodiments, the 3′ terminal position of the second region of linked nucleosides does not contain a 2′-O-methyl modification.

In certain embodiments, sugars of the nucleosides at any of positions 2 and 14 downstream from the first nucleoside of the 5′ region of the first region of linked nucleosides contain 2′-F modifications.

In certain embodiments, sugars of the nucleosides of the second region of linked nucleosides that correspond in position to any of the nucleosides of the first region of linked nucleosides at any of positions 11 to 13 downstream from the first nucleoside of the 5′ region of the first region of linked nucleosides contain 2′-F modifications.

In certain embodiments, the 3′ terminal nucleoside of the second region of linked nucleosides contains a 2′-F modification.

In certain embodiments, one or more of the odd numbered nucleosides starting from the 5′ region of the first region of linked nucleosides may be modified, and/or wherein one or more of the even numbered nucleosides starting from the 5′ region of the first region of linked nucleosides may be modified, wherein typically the modification of the even numbered nucleosides is a second modification that is different from the modification of odd numbered nucleosides.

In certain embodiments, one or more of the odd numbered nucleosides starting from the 3′ region of the second region of linked nucleosides may be modified by a modification that is different from the modification of odd numbered nucleosides of the first region of linked nucleosides.

In certain embodiments, one or more of the even numbered nucleosides starting from the 3′ region of the second region of linked nucleosides is modified by a modification that is different from the modification of even numbered nucleosides of the first region of linked nucleoside.

In certain embodiments, at least one or more of the modified even numbered nucleosides of the first region of linked nucleosides is adjacent to at least one or more of the differently modified odd numbered nucleosides of the first nucleoside region.

In certain embodiments, at least one or more of the modified even numbered nucleosides of the second nucleoside region is adjacent to at least one or more of the differently modified odd numbered nucleosides of the second region of linked nucleosides.

In certain embodiments, sugars of one or more of the odd numbered nucleosides starting from the 5′ region of the first region of nucleosides may be 2′-O-methyl modified sugars.

In certain embodiments, one or more of the even numbered nucleosides starting from the 3′ region of the first region of linked nucleosides may be 2′-F modified sugars.

In certain embodiments, sugars of one or more of the odd numbered nucleosides starting from the 5′ region of the second region of linked nucleosides may be 2′-O methyl modified sugars.

In certain embodiments, one or more of the even numbered nucleosides starting from the 5′ region of the second region of linked nucleosides may be 2′-F modified sugars.

In certain embodiments, sugars of a plurality of adjacent nucleosides of the first nucleoside region may be modified by a common or different modification.

In certain embodiments, sugars of a plurality of adjacent nucleosides of the second nucleoside region may be modified by a common or different modification.

In certain embodiments, sugars of a plurality of adjacent nucleosides of the hairpin loop may be modified by a common or different modification. The common modification may be a 2′-F modified sugar.

Alternatively, the common modification may be a 2′-O-methyl modified sugar.

The plurality of adjacent 2′-O-methyl modified sugars may be present in at least eight adjacent nucleosides of the first and/or second nucleoside regions. The plurality of adjacent 2′-O-methyl modified sugars may be present in three or four adjacent nucleosides of the hairpin loop.

In certain embodiments, wherein the hairpin loop, as disclosed earlier herein, comprises at least one nucleoside having a modified sugar.

In certain embodiments, the at least one nucleoside is adjacent to a nucleoside with a differently modified sugar, wherein optionally all adjacent nucleosides in the hairpin loop have a differently modified sugar.

In certain embodiments, the modified sugar is a 2′-O-methyl modified sugar, and the differently modified sugar is a 2′-F modified sugar.

In certain embodiments one or more nucleosides of the first region of linked nucleosides and/or the second region of linked nucleosides may be an inverted nucleoside and is attached to an adjacent nucleoside via the 3′ carbon of its sugar and the 3′ carbon of the sugar of the adjacent nucleoside, and/or one or more nucleosides of the first region of linked nucleosides and/or the second region of linked nucleosides is an inverted nucleoside and is attached to an adjacent nucleoside via the 5′ carbon of its sugar and the 5′ carbon of the sugar of the adjacent nucleoside.

### Murna Nucleic Acid Constructs

According to a second aspect, the present disclosure is directed to a nucleic acid construct comprising at least:
(a) a first nucleic acid portion that is at least partially complementary to at least a first portion of an RNA, which is transcribed from an C5 gene;
(b) a second nucleic acid portion that is at least partially complementary to at least a second portion of an RNA, which is transcribed from a C5 gene, the second portion being different from the first portion;
(c) a third nucleic acid portion that is at least partially complementary to the first nucleic acid portion of (a), so as to form a first nucleic acid duplex region therewith;
(d) a fourth nucleic acid portion that is at least partially complementary to the second nucleic acid portion of (b), so as to form a second nucleic acid duplex region therewith.

The construct may be designed such that subsequent to in vivo administration the construct disassembles to yield at least first and second discrete nucleic acid targeting molecules that respectively target the RNA portions transcribed from the target genes of (a) and (b);

whereby (i) the first nucleic acid targeting molecule is capable of modulating expression of the target gene of (a), and comprises, or is derived from, at least the first nucleic acid portion of (a), and (ii) the second nucleic acid targeting molecule is capable of modulating expression of the target gene of (b), and comprises, or is derived from, the second nucleic acid portion of (b).

The construct may be designed to disassemble such that the first and second discrete nucleic acid targeting molecules are respectively processed by independent RNAi-induced silencing complexes.

### Sequence Features, Labile Functionality and Structural Features of the RNA Molecules

The construct according to the second aspect and its aforementioned embodiments may at least comprise one labile functionality such that subsequent to in vivo administration the construct is cleaved so as to yield the at least first and second discrete nucleic acid targeting molecules.

The labile functionality comprises one or more unmodified nucleotides. In particular the one or more unmodified nucleotides of the labile functionality represent one or more cleavage positions within the construct whereby subsequent to in vivo administration the construct is cleaved at the one or more cleavage positions so as to yield the at least first and second discrete nucleic acid targeting molecules. Especially the cleavage positions may be respectively located within the construct so that subsequent to cleavage the first discrete nucleic acid targeting molecule comprises, or is derived from, the first nucleic acid duplex region, and the second discrete nucleic acid targeting molecule comprises, or is derived from, the second nucleic acid duplex region. Optionally, the first discrete nucleic acid targeting molecule comprises or consists of the first nucleic acid portion of (a) and the third nucleic acid portion of (c), and/or the second discrete nucleic acid targeting molecule comprises or consists of the second nucleic acid portion of (b) and the fourth nucleic acid portion of (d).

In certain embodiments
(a) the first nucleic acid portion has a nucleobase sequence selected from SEQ ID NOs: 1 to 250 in Table 1a;
(b) the second nucleic acid portion has a nucleobase sequence selected from Table 1a (SEQ ID NOs: 1 to 250);
(c) the third nucleic acid portion has a nucleobase sequence selected from Table 1b SEQ ID NOs: 251 to 500; and/or
(d) the fourth nucleic acid portion has a nucleobase sequence selected from Table 1b (SEQ ID NOs: 251 to 500).

wherein the third and fourth nucleobase sequences, to the extent they have a length of 14 nucleobases, may be shorter by one, two or three nucleobases, wherein optionally the 5′-terminal nucleobase(s) is/are absent.

In certain such embodiments, the first nucleic acid portion of (a) may be directly or indirectly linked to the fourth nucleic acid portion of (d) as a primary structure.

In certain embodiments, the first and the fourth nucleic acid portions have the nucleobase sequences of SEQ ID NOs: 30 and 287, 30 and 333, 37 and 280, 37 and 333, 83 and 280, 83 and 287 and, respectively, optionally, wherein the sequences of SEQ ID NOs: 280, 287, and 333 may be shorter by one, two, three or four nucleobases, wherein optionally the 5′-terminal nucleobase(s) is/are absent.

In certain embodiments, the second nucleic acid portion of (b) may be directly or indirectly linked to the third nucleic acid portion of (c) as a primary structure.

In certain embodiments, the second and third nucleic acid portions have the nucleobase sequences of SEQ ID NOs: 30 and 287, 30 and 333, 37 and 280, 37 and 333, 83 and 280, 83 and 287 and, respectively, optionally, wherein the sequences of SEQ ID NOs: 280, 287, and 333 may be shorter by one, two, three or four nucleobases, wherein optionally the 5′-terminal nucleobase(s) is/are absent.

In certain embodiments, the construct may further comprise 1 to 8 additional nucleic acid portions that are respectively at least partially complementary to an additional 1 to 8 portions of RNA transcribed from one or more target genes, which target genes may be the same or different to each other, and/or the same or different to the target genes defined in (a) and/or (b), and wherein each of the 1 to 8 additional nucleic acid portions respectively form additional duplex regions with respective passenger nucleic acid portions that are respectively at least partially complementary therewith. In particular, the second nucleic acid portion of (b), and the 1 to 8 additional nucleic acid portions, may be directly or indirectly linked to selected passenger nucleic acid portions as respective primary structures.

In certain embodiments the direct or indirect linking may represent either (i) an internucleotide bond, (ii) an internucleotide nick, or (iii) a nucleic acid linker portion of 1, 2, 3, 4, 5, 6, 7, 8, 9, or 10 nucleotides, the nucleic acid linker optionally being single stranded. Optionally, the linking may be direct, thereby giving rise to (a) contiguous strand(s).

In certain embodiments, there may exist some complementarity between the first nucleic acid portion of (a) and the second nucleic acid portion of (b), or the third nucleic acid portion of (c) and the fourth nucleic acid portion of (d). Optionally the complementarity
(i) may be 1, 2, 3, 4, 5, 6, 7, 8, 9, or 10, optionally 2, 3, 4 or 5 base pairs; and/or
(ii) may be between the first nucleic acid portion of (a) and the second nucleic acid portion of (b).

In certain embodiments, the internucleotide bond may involve at least one of the one or more unmodified nucleotides, wherein optionally cleavage may occur at the 3′ position of (at least one of) the unmodified nucleotide(s).

In certain embodiments, the first nucleic acid portion of (a), and/or the second nucleic acid portion of (b), and/or the third nucleic acid portion of (c), and/or the fourth nucleic acid portion of (d), may be respectively 7 to 25 nucleotides in length. Optionally, the first nucleic acid portion of (a) and/or the second nucleic acid portion of (b) may have a length of 18 to 21, more optionally 18 to 20, and yet more optionally 19 nucleotides. In optional embodiments, the first nucleic acid portion of (a) and the second nucleic acid portion of (b) have a length of 19 nucleotides. It may be further optional that the third nucleic acid portion of (c), and/or the fourth nucleic acid portion of (d) have a length of 11 to 20, more optionally 13 to 16, and yet more optionally 14 or 15, most optionally 14 nucleotides.

In certain embodiments, the first nucleic portion of (a) and the second nucleic acid portion of (b) may have a length of 19 nucleotides and the third nucleic acid portion of (c) as well as the fourth nucleic acid portion of (b) may have a length of 14 nucleotides.

In certain embodiments, the unmodified nucleotide(s) is/are at any of position 18 to 25, more optionally at any of positions 18 to 21, and/or the 3′ terminal position of the first nucleic acid portion of (a) and/or of the third nucleic acid portion of (c).

In certain embodiments, wherein the unmodified nucleotide is at position 19.

In certain embodiments, the first nucleic portion of (a) and the second nucleic acid portion of (b) may have a length of 19 nucleotides and the third nucleic acid portion of (c) as well as the fourth nucleic acid portion of (b) may have a length of 14 nucleotides and the unmodified nucleoside is at position 19 of the first nucleic acid portion of (a) and the second nucleic acid portion of (b).

In certain embodiments, the nucleic acid linker portion may be 1 to 8 nucleotides in length, optionally 2 to 7 or 3 to 6 nucleotides in length, more optionally about 4 or 5 and most optionally 4 nucleotides in length.

In certain embodiments, one, more of all of the duplex regions independently may have a length of 10 to 19, more optionally 13 to 19, and yet more optionally 13, 14 or 15 base pairs, most optionally 14 base pairs, wherein optionally there is one mismatch within the duplex region.

In certain embodiments, the nucleic acid construct may be blunt ended.

In certain embodiments,
the first nucleic acid portion of (a); and/or
the second nucleic acid portion of (b); and/or
the third nucleic acid portion of (c); and/or
the fourth nucleic acid portion of (d); and/or
to the extent present, the 1 to 8 additional nucleic acid portions as defined previously herein; and/or
to the extent present, the passenger nucleic acid portions as defined previously herein;
may have an overhang.

In certain embodiments, the target RNA may be an mRNA or another RNA molecule.

### Ligands

The nucleic acid construct according to the second aspect and the aforementioned embodiments may further comprise one or more ligands.

In certain embodiments, the first nucleic acid portion of (a), and/or the second nucleic acid portion of (b), and/or the third nucleic acid portion of (c), and/or the fourth nucleic acid portion of (d), and/or, to the extent present, the 1 to 8 additional nucleic acid portions as defined previously herein, and/or the passenger nucleic acid portions as defined previously herein, respectively may have a 5′ to 3′ directionality thereby defining 5′ and 3′ regions thereof.

In certain embodiments, one or more ligands are conjugated at the 3′ region, optionally the 3′ end, of any of (i) the third nucleic acid portion of (c), and/or (ii) the fourth nucleic acid portion of (d), and/or, to the extent present, the (iii) passenger nucleic acid portions as defined previously herein.

In certain embodiments, one or more ligands may be conjugated at one or more regions intermediate of the 5′ and 3′ regions of any of the nucleic acid portions, optionally of the third nucleic acid portion of (c), and/or the fourth nucleic acid portion of (d), and/or the passenger nucleic acid portions as defined previously herein.

In certain embodiments, one or more ligands may be conjugated at the 5′ region, optionally the 5′ end, of any of the nucleic acid portions.

In certain embodiments, the one or more ligands may be any cell directing moiety, such as lipids, carbohydrates, aptamers, vitamins and/or peptides that bind cellular membrane or a specific target on cellular surface. In an optional embodiment, the one or more carbohydrates can be a monosaccharide, disaccharide, trisaccharide, tetrasaccharide, oligosaccharide or polysaccharide. In a more optional embodiment, the one or more carbohydrates comprises one or more hexose moieties. Especially, the one or more hexose moieties may be one or more galactose moieties, one or more lactose moieties, one or more N-Acetyl-Galactosamine moieties, and/or one or more mannose moieties. The hexose moiety may comprise two or three N-Acetyl-Galactosamine moieties. In particular, the hexose moiety comprises three N-Acetyl-Galactosamine moieties.

In certain embodiments, the one or more ligands may be attached in a linear configuration, or in a branched configuration. Optionally, wherein the one or more ligands may be attached as a biantennary or triantennary configuration, or as a configuration based on single ligands at different positions.

Optionally, the ligand may have the following structure:

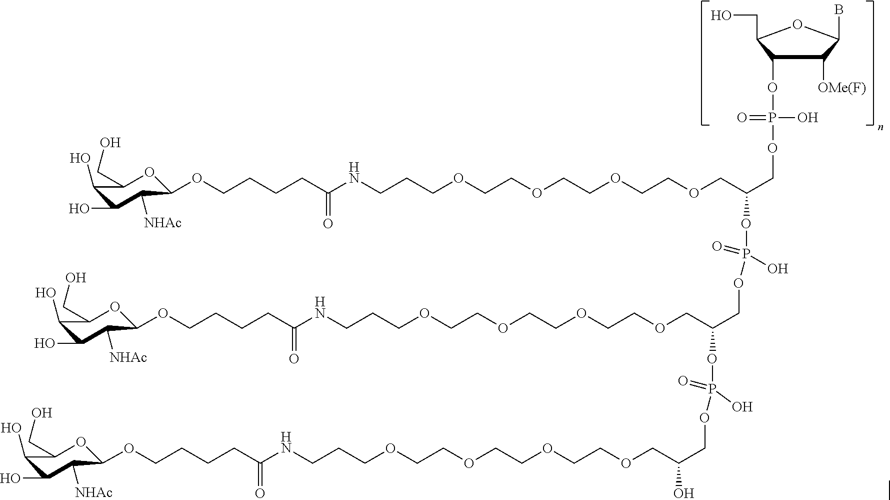

### Internucleoside Linkages

The nucleotide construct according to the second aspect of the present disclosure or its aforementioned embodiments comprises one or more phosphorothioate or phosphorodithioate internucleotide linkages.

In certain embodiments, the nucleic acid construct comprises 1 to 15 phosphorothioate or phosphorodithioate internucleotide linkages.

In certain embodiments, the nucleic acid construct comprises one or more phosphorothioate or phosphorodithioate internucleotide linkages at one or more of the 5′ and/or 3′ regions of the first nucleic acid portion of (a), and/or the second nucleic acid portion of (b), and/or the third nucleic acid portion of (c), and/or the fourth nucleic acid portion of (d), and/or the 1 to 8 additional nucleic acid portions as defined previously herein, and/or the passenger nucleic acid portions as defined in previously herein.

In certain embodiments, the nucleic acid construct comprises phosphorothioate or phosphorodithioate internucleotide linkages between at least two adjacent nucleotides of the nucleic acid linker portion as defined in previously herein.

In certain embodiments, the nucleic acid construct comprises a phosphorothioate or phosphorodithioate internucleotide linkage between each adjacent nucleotide that is present in the nucleic acid linker portion.

In certain embodiments, the nucleic acid construct comprises a phosphorothioate or phosphorodithioate internucleotide linkage linking:
the first nucleic acid portion of (a) to the nucleic acid linker portion as defined in previously herein; and/or
the second nucleic acid portion of (b) to the nucleic acid linker portion as defined previously herein; and/or
the third nucleic acid portion of (c) to the nucleic acid linker portion as defined previously herein and/or
the fourth nucleic acid portion of (d) to the nucleic acid linker portion as defined previously herein; and/or
the 1 to 8 additional nucleic acid portions as defined previously herein to the nucleic acid linker portion as further defined previously herein; and/or
the passenger nucleic acid portions as defined previously herein to the nucleic acid linker portion as further defind previously herein.

### Modifications

In the nucleic acid construct according to the second aspect of the present disclosure and its aforementioned embodiments, at least one nucleotide of at least one of the following may be modified:
the first nucleic acid portion of (a); and/or
the second nucleic acid portion of (b); and/or
the third nucleic acid portion of (c); and/or
the fourth nucleic acid portion of (d); and/or
to the extent present, the 1 to 8 additional nucleic acid portions as defined previously herein; and/or
to the extent present, the passenger nucleic acid portions as defined previously herein; and/or
to the extent present, the nucleic acid linker portion as further defined previously herein.

In an optional embodiment, one or more of the odd numbered nucleotides starting from the 5′ region of one of the following may be modified, and/or wherein one or more of the even numbered nucleotides starting from the 5′ region of one of the following are modified, wherein typically the modification of the even numbered nucleotides is a second modification that is different from the modification of odd numbered nucleotides:
the first nucleic acid portion of (a); and/or
the second nucleic acid portion of (b); and/or
the third nucleic acid portion of (c); and/or
the fourth nucleic acid portion of (d); and/or
to the extent present, the 1 to 8 additional nucleic acid portions as defined previously herein; and/or
to the extent present, the passenger nucleic acid portions as defined previously herein.

In certain embodiments, one or more of the odd numbered nucleotides starting from the 3′ region of the third nucleic acid portion of (c) may be modified by a modification that is different from the modification of odd numbered nucleotides starting from the 5′ region of the first nucleic acid portion of (a); and/or
one or more of the odd numbered nucleotides starting from the 3′ region of the fourth nucleic acid portion of (d) may be modified by a modification that is different from the modification of odd numbered nucleotides starting from the 5′ region of the second nucleic acid portion of (b); and/or
one or more of the odd numbered nucleotides starting from the 3′ region of the passenger nucleic acid portions as defined previously herein, to the extent present, may be modified by a modification that is different from the modification of odd numbered nucleotides starting from the 5′ region of the 1 to 8 additional nucleic acid portions as defined previously herein; and/or
wherein one or more of the nucleotides of a nucleic acid linker portion as further defined previously herein, to the extent present, may be modified by a modification that (i) is different from the modification of an adjacent nucleotide of the 3′ region of the first nucleic acid portion of (a); and/or (ii) is different from the modification of an adjacent nucleotide of the 3′ region of the second nucleic acid portion of (b); and/or is different from the modification of an adjacent nucleotide of the 3′ region of the 1 to 8 additional nucleic acid portions, to the extent present, as defined previously herein.

In certain embodiments, one or more of the even numbered nucleotides starting from the 3′ region of: (i) the third nucleic acid portion of (c), and/or (ii) the fourth nucleic acid portion of (d), and/or (iii) the passenger nucleic acid portions as defined previously herein, to the extent present, may be modified by a modification that is different from the modification of odd numbered nucleotides starting from the 3′ region of these respective portions.

In certain embodiments, at least one or more of the modified even numbered nucleotides of (i) the first nucleic acid portion of (a), and/or (ii) the second nucleic acid portion of (b), and/or (iii), to the extent present, the 1 to 8 additional nucleic acid portions as defined previously herein, may be adjacent to at least one or more differently modified odd numbered nucleotides of these respective portions.

In certain embodiments, at least one or more of the modified even numbered nucleotides of (i) the third nucleic acid portion of (c), and/or (ii) the fourth nucleic acid portion of (d), and/or (iii), to the extent present, the passenger nucleic acid portions as defined previously herein, may be adjacent to at least one or more differently modified odd numbered nucleotides of these respective portions.

In certain embodiments, a plurality of adjacent nucleotides of (i) the first nucleic acid portion of (a), and/or (ii) the second nucleic acid portion of (b), and/or (iii), to the extent present, the 1 to 8 additional nucleic acid portions as defined previously herein, may be modified by a common modification.

In certain embodiments, a plurality of adjacent nucleotides of (i) the third nucleic acid portion of (c), and/or (ii) the fourth nucleic acid portion of (d), and/or (iii), to the extent present, the passenger nucleic acid portions as defined previously herein, may be modified by a common modification.

In certain embodiments, the plurality of adjacent commonly modified nucleotides may be 2 to 4 adjacent nucleotides, optionally 3 or 4 adjacent nucleotides.

In certain embodiments, the plurality of adjacent commonly modified nucleotides may be located in the 5′ region of (i) the third nucleic acid portion of (c), and/or (ii) the fourth nucleic acid portion of (d), and/or (iii), to the extent present, the passenger nucleic acid portions previously herein.

In certain embodiments, a plurality of adjacent commonly modified nucleotides may be located in the nucleic acid linker portion as further defined previously herein.

In certain embodiments, the one or more of the modified nucleotides of first nucleic acid portion of (a) may not have a common modification present in the corresponding nucleotide of the third nucleic acid portion of (c) of the first duplex region; and/or one or more of the modified nucleotides of second nucleic acid portion of (b) may not have a common modification present in the corresponding nucleotide of the fourth nucleic acid portion of (d) of the second duplex region; and/or one or more of the modified nucleotides of the 1 to 8 additional nucleic acid portions, to the extent present, as defined previously herein, may not have a common modification present in the corresponding nucleotide of the corresponding passenger nucleic acid portions of the respective duplex regions.

In certain embodiments, the one or more of the modified nucleotides of the first nucleic acid portion of (a) may be shifted by at least one nucleotide relative to a commonly modified nucleotide of the third nucleic acid portion of (c); and/or one or more of the modified nucleotides of the second nucleic acid portion of (b) may be shifted by at least one nucleotide relative to a commonly modified nucleotide of the fourth nucleic acid portion of (d); and/or one or more of the modified nucleotides of the 1 to 8 additional nucleic acid portions, to the extent present, as defined previously herein may be shifted by at least one nucleotide relative to a commonly modified nucleotide of the passenger nucleic acid portions, to the extent present, as defined previously herein.

In certain embodiments, the modification and/or modifications may be each and individually sugar, phosphate, or base modifications.

In certain embodiments, the modification may be selected from nucleotides with 2′ modified sugars; conformationally restricted nucleotides (CRN) sugar such as locked nucleic acid (LNA), (S)-constrained ethyl bicyclic nucleic acid, and constrained ethyl (cEt), tricyclo-DNA; morpholino, unlocked nucleic acid (UNA), glycol nucleic acid (GNA), D-hexitol nucleic acid (HNA), and cyclohexene nucleic acid (CeNA). In optional embodiments, wherein the 2′ modified sugar may be selected from 2′-O-alkyl modified sugar, 2′-O-methyl modified sugar, 2′-O-methoxyethyl modified sugar, 2′-O-allyl modified sugar, 2′-C-allyl modified sugar, 2′-deoxy modified sugar such as 2′-deoxy ribose, 2′-F modified sugar, 2′-arabino-fluoro modified sugar, 2′-O-benzyl modified sugar, 2′-amino modified sugar, and 2′-O-methyl-4-pyridine modified sugar.

In certain embodiments, the base modification may be any one of an abasic nucleotide and a non-natural base comprising nucleotide.

In certain embodiments, at least one modification may be a 2′-O-methyl modification in a ribose moiety.

In certain embodiments, at least one modification may be a 2′-F modification in a ribose moiety.

In certain embodiments, the nucleotides at any of positions 2 and 14 downstream from the first nucleotide of the 5′ region of (i) the first nucleic acid portion of (a); and/or (ii) the second nucleic acid portion of (b); and/or (iii), to the extent present, the 1 to 8 additional nucleic acid portions as defined previously herein; may not contain 2′-O-methyl modifications in ribose moieties.

In certain embodiments, one, two or all three nucleotides of (i) the third nucleic acid portion of (c); and/or (ii) the fourth nucleic acid portion of (d); and/or (iii), to the extent present, the passenger nucleic acid portions as defined previously herein; that respectively correspond in position to any of the nucleotides at any of positions 11 to 13 downstream from the first nucleotide of the 5′ region of (i) the first nucleic acid portion of (a); and/or (ii) the second nucleic acid portion of (b); and/or (iii) the 1 to 8 additional nucleic acid portions, to the extent present, as defined previously herein; may not contain 2′-O-methyl modifications in ribose moieties.

In certain embodiments, the nucleotides at any of positions 2 and 14 downstream from the first of (i) the first nucleic acid portion of (a); and/or (ii) the second nucleic acid portion of (b); and/or (iii), to the extent present, the 1 to 8 additional nucleic acid portions as defined previously herein; may contain 2′-F modifications in ribose moieties.

In certain embodiments, one, two or all three nucleotides of (i) the third nucleic acid portion of (c); and or (ii) the fourth nucleic acid portion of (d); and/or (iii), to the extent present, the passenger nucleic acid portions as defined previously herein; that respectively correspond in position to any of the nucleotides at any of positions 11 to 13 downstream from the first nucleotide of the 5′ region of (i) the first nucleic acid portion of (a); and/or (ii) the second nucleic acid portion of (b); and/or (iii), to the extent present, the 1 to 8 additional nucleic acid portions as defined previously herein; may contain 2′-F modifications in ribose moieties.

In certain embodiments, all remaining nucleotides may contain either 2′-O-methyl modifications or 2′-F modifications in ribose moieties, optionally with the exception of the unmodified nucleotide(s) in accordance with the labile linkage defined herein. Optionally, the remaining nucleotides may contain 2′-O-methyl modifications in ribose moieties.

In certain embodiments, the one or more, optionally one, unmodified nucleotide represents any of the nucleotides of the nucleic acid linker portion as further defined previously herein, optionally the nucleotide of the nucleic acid linker portion as further defined previously herein that is adjacent to (i) the third nucleic acid portion of (c); and or (ii) the fourth nucleic acid portion of (d); and/or (iii), to the extent present, the passenger nucleic acid portions as defined previously herein.

In certain embodiments,
(a) the first nucleic acid portion may be selected from Table 3a;
(b) the second nucleic acid portion may be selected from Table 3a;
(c) the third nucleic acid portion may be selected from Table 3b; and/or
(d) the fourth nucleic acid portion may be selected from Table 3b.

In optional embodiments, the first nucleic acid portion and the second nucleic acid portion may be selected from Table 3a, wherein the first and second nucleic acid portions are different; and the third and fourth nucleic acid portions may be selected from Table 3b.

In certain embodiments, the 3′ terminal positions of the first and the third nucleic acid portions may be replaced with an unmodified nucleotide.

In certain embodiments, the nucleic acid construct comprises at least one vinylphosphonate modification, such as at least one vinylphosphonate modification in the 5′ region of (i) the first nucleic acid portion of (a); and/or (ii) the second nucleic acid portion of (b); and/or (iii), to the extent present, the 1 to 8 additional nucleic acid portions as defined previously herein.

In certain embodiments, one or more nucleotides of
the first nucleic acid portion of (a); and/or
the second nucleic acid portion of (b); and/or
the third nucleic acid portion of (c); and/or
the fourth nucleic acid portion of (d); and/or
to the extent present, the 1 to 8 additional nucleic acid portions as defined previously herein; and/or
to the extent present, the passenger nucleic acid portions as defined previously herein;
may be an inverted an inverted nucleotide and may be attached to the adjacent nucleotide via the 3′ carbon of the nucleotide and the 3′ carbon of the adjacent nucleotide, and/or may be an inverted nucleotide and may be attached to the adjacent nucleotide via the 5′ carbon of the nucleotide and the 5′ carbon of the adjacent nucleotide.

In certain embodiments, the inverted nucleotide may be attached to the adjacent nucleotide via a phosphate group by way of a phosphodiester linkage; or may be attached to the adjacent nucleotide via a phosphorothioate group; or may be attached to the adjacent nucleotide via a phosphorodithioate group.

Compositions and Pharmaceutical Compositions Including shRNA, mxRNA and/or muRNA Oligomeric Constructs

According to a third aspect, the present disclosure is directed to a composition comprising an oligomeric compound according to the first aspect and/or a nucleic acid construct according the second aspect of the present disclosure, and a physiologically acceptable excipient.

According to a fourth aspect, the present disclosure is directed to pharmaceutical composition comprising an oligomeric compound according to the first aspect and/or a nucleic acid construct according to the second aspect of the present disclosure.

The pharmaceutical composition may further comprise a pharmaceutically acceptable excipient, diluent, antioxidant, and/or preservative.

The oligomeric compound according to the first aspect and/or the construct according to the second aspect may be the only pharmaceutically active agent(s).

Alternatively, the pharmaceutical composition furthermore comprises one or more further pharmaceutically active agents. The further pharmaceutically active agent(s) is/are (an) agent(s) which modulate(s) the innate and/or the adaptive immune system, for example a further oligomeric compound which is directed to an immune system target different from complement component C5, optionally Interleukin-6; agents lowering the expression or level of Interleukin-6; or an agent such as an antibody targeting a complement component, the antibody optionally being Eculizumab. Optionally the oligomeric compound and/or the nucleic acid construct; and the further pharmaceutically active agent(s) are to be administered concomitantly or in any order.

Diseases to be Treated by shRNA, mxRNA and/or muRNA Oligomeric Compounds and Further Uses

According to a fifth aspect, the present disclosure is directed to an oligomeric compound according to the first aspect and/or a nucleic acid construct according to the second aspect of the present disclosure, for use in human or veterinary medicine or therapy.

According to a sixth aspect, the present disclosure is directed to an oligomeric compound according to the first aspect and/or a nucleic acid construct according to the second aspect of the present disclosure, for use in a method of treating, ameliorating and/or preventing a disease or disorder.

The disease or disorder may be a disease or disorder associated C5 or a disease or disorder requiring reduction of C5 expression.

In particular, the disease or disorder is selected from the group consisting of autoimmune disease, complement system dysfunction including aberrant upregulation of complement components such as C5, age-related macular degeneration (AMD) including dry AMD and geographic atrophy, paroxysmal nocturnal hemoglobinuria (PNH), Generalized myasthenia gravis (gMG), Lupus nephritis (LN), Alzheimer's disease, Atherosclerosis, Inflammation of the choroid plexus, atypical hemolytic uremic syndrome (aHUS), C3 glomerulopathy (C3G), Ig-mediated kidney pathologies such as IgA nephropathy and primary membranous nephropathy, asthma, rheumatic disease, rheumatoid arthritis, systemic lupus crythematosus (SLE), anti-neutrophil cytoplasmic antibody (ANCA) vasculitis, antiphospholipid antibody syndrome (APS), glomerulonephritis, dermatomyositis bullous pemphigoid, Shiga toxin E. coli-related hemolytic uremic syndrome, amyotrophic lateral sclerosis (ALS), Central nervous system (CNS) diseases, myasthenia gravis (MG), neuromyelistis optica (NMO), dense deposit disease, C3 neuropathy, cold agglutinin disease, humoral and vascular transplant rejection, graft dysfunction, myocardial infarction, asthma, rheumatoid arthritis (RA) sensitization towards a transplant, antiphospho lipid antibody syndrome; lupus nephritis; ischemia-reperfusion injury; typical or infectious hemolytic uremic syndrome (tHUS); dense deposit disease (DDD); neuromyelitis optica (N O); multifocal motor neuropathy (MMN); multiple sclerosis (MS); macular degeneration (e.g., age-related macular degeneration (AM D)); hemolysis, elevated liver enzymes, and low platelets (HELLP) syndrome; thrombotic thrombocytopenia purpura (TTP); spontaneous fetal loss; Pauci-immune vasculitis; epidermolysis bullosa; recurrent fetal loss; pre-eclampsia, traumatic brain injury, myasthenia gravis, cold agglutinin disease, dermatomyositis bullous pemphigoid, Shiga toxin E. co/-related hemolytic uremic syndrome, C3 nephropathy, anti-neutrophil cytoplasmic antibody-associated vasculitis, humoral and vascular transplant rejection, graft dysfunction, myocardial infarction, an allogenic transplant, sepsis, Coronary artery disease, dermatomyositis, Graves' disease, atherosclerosis, systemic inflammatory response sepsis, septic shock, spinal cord injury, glomerulonephritis, Hashimoto's thyroiditis, type I diabetes, pemphigus, autoimmune hemolytic anemia (AIHA), ITP, Goodpasture syndrome, Degos disease, antiphospholipid syndrome (APS), catastrophic APS (CAPS), a cardiovascular disorder, myocarditis, a cerebrovascular disorder, a peripheral vascular disorder, a renovascular disorder, a mesenteric/enteric vascular disorder, vasculitis, Henoch-Schonlein purpura nephritis, systemic lupus erythematosus-associated vasculitis, vasculitis associated with rheumatoid arthritis, immune comple vasculitis, Takayasu's disease, dilated cardiomyopathy, diabetic angiopathy, Kawasaki's disease (arteritis), venous gas embolus (VGE), and restenosis following stent placement, rotational atherectomy, membrancous nephropathy, Guiliain-Barre syndrome, and percutaneous transluminal coronary angioplasty Age-related macular degeneration (AMD) and/or Geographic atrophy (GA); Uveitis and/or panuveitis; Cold agglutinin disease, Membranoproliferative glomerulonephritis (MPGN), Guillain-Barré syndrome, Shiga toxin-producing E. coli hemolytic-uremic syndrome (STEC-HUS), organ transplantation-associated autoimmune diseases, and sepsis. According to a seventh aspect, the present disclosure is directed to a method of treating a disease or disorder comprising administration of an oligomeric compound according the first aspect and/or a nucleic acid construct according to the second aspect of the present disclosure, to an individual in need of treatment.

In particular, the disease of disorder is selected from paroxysmal nocturnal hemoglobinuria (PNH), Alzheimer's disease, Atherosclerosis, Inflammation of the choroid plexus, Generalized myasthenia gravis (gMG), amyotrophic lateral sclerosis (ALS), Lupus nephritis (LN), Central nervous system (CNS) diseases; Age-related macular degeneration (AMD) and/or Geographic atrophy (GA); Uveitis and/or panuveitis; Cold agglutinin disease, Membranoproliferative glomerulonephritis (MPGN), Guillain-Barré syndrome, Shiga toxin-producing E. coli hemolytic-uremic syndrome (STEC-HUS) and organ transplantation-associated autoimmune diseases.

In certain embodiments, the nucleic acid construct and/or the oligomeric compound is administered at a dose of about 0.05 mg/kg to about 50.0 mg/kg, optionally 0.05 mg/kg to about 30.0 mg/kg or 10.0 mg/kg to about 50.0 mg/kg of body weight of the human subject. In other words, the mass indicated in “mg” is the mass of administred nucleic acid construct and/or oligomeric compound and the mass indicated in “kg” is the kilogram bodyweight of the human subjects to which the “mg” mass refers.

The oligomeric compound and/or the nucleic acid construct may be administered subcutaneously or intravenously to the individual.

According to an eighth aspect, the present disclosure is directed to a use of an oligomeric compound according to the first aspect or a nucleic acid construct according to the second aspect, for use in research as a gene function analysis tool.

According to a ninth aspect, the present disclosure is directed to a use of an oligomeric compound according to the first aspect and/or a nucleic acid construct according to the second aspect in the manufacture of a medicament for a treatment of a disease or disorder.

### Constructs and Sequences of the Oligomeric Compounds

The following Tables show nucleobase sequences of antisense and sense strands of oligomeric compounds of the disclosure as well as of nucleobase sequences of single-stranded oligomeric compounds of the disclosure, and definitions of modified oligomeric compounds of the disclosure (the notation including nucleobase sequence, sugar modifications, and, where applicable, modified phosphates).

The notation used is common in the art and as the following meaning:
A represents adenine;
U represents uracil;
C represents cytosine;
G represents guanine.
5Phos represents a 5′ terminal phosphate group which is optional but not indispensable;
m represents a methyl modification at the 2′ position of the sugar of the underlying nucleoside;
f represents a fluoro modification at the 2′ position of the sugar of the underlying nucleoside;
r indicates an unmodified (2′-OH) ribonucleotide;
[Ps] or #represents a phosphorothioate inter-nucleoside linkage;
i represents an inverted inter-nucleoside linkage, which can be either 3′-3′, or 5′-5′;
3×GalNAc represents a trivalent GalNAc.

Tables 1a and 1b below show nucleobase sequences of antisense and sense strands of 250 oligomeric compounds in accordance with the Examples.

TABLE 1a
Nucleobase sequences of the antisense strands of
250 constructs of the disclosure

| | Antisense | |
|---|---|---|
| SEQ ID NO: | ID | 19 mer Antisense |
| 1 | 24151 | ACACAGUUUGGCCUGGAGA |
| 2 | 24152 | GGAAUCUUGAAGUCAGGAA |
| 3 | 24153 | CUGGGCUUGUAGCUGGCAC |
| 4 | 24154 | UCAAGUAAUUAUAGUGAGU |
| 5 | 24155 | AAACAGGUUUGUCUGUAUG |
| 6 | 24156 | GCAGACAUUUUAACACAGA |
| 7 | 24157 | ACCUGGAGCUGGUUGCCAC |
| 8 | 24158 | GAUAAAAUCAAGUAAUUAU |
| 9 | 24159 | GACACAGUUUGGCCUGGAG |
| 10 | 24160 | CGGAAUCUUGAAGUCAGGA |
| 11 | 24161 | AGACAUUUUAACACAGAAC |
| 12 | 24162 | UGAAGUCAGGAAAAGAGAU |
| 13 | 24163 | CACAGUUUGGCCUGGAGAA |
| 14 | 24164 | AAUUAUAGUGAGUUAUUUU |
| 15 | 24165 | UCCAAGUCAGAUGUCUCUU |
| 16 | 24166 | UCAGGAAAAGAGAUAAUUC |
| 17 | 24167 | GGCAAGACAUAUUCUUUAA |
| 18 | 24168 | GAAGGCCAAUUUCCAGAGG |
| 19 | 24169 | CAAGUAAUUAUAGUGAGUU |
| 20 | 24170 | AUAAAAUCAAGUAAUUAUA |
| 21 | 24171 | AUCAAGUAAUUAUAGUGAG |
| 22 | 24172 | GCCAAUUUCCAGAGGAAGC |
| 23 | 24173 | AGUAAUUAUAGUGAGUUAU |
| 24 | 24174 | UAAAGGUACUUGUUGUUUA |
| 25 | 24175 | GACUGCUGUUUCAGAAUCA |
| 26 | 24176 | ACUGCUGUUUCAGAAUCAA |
| 27 | 24177 | AUAUAAAGGUACUUGUUGU |
| 28 | 24178 | UGUAAACAGUUCCUUUCAA |
| 29 | 24179 | GGUAACUUUGGCUGAGAGA |
| 30 | 24180 | UAUAGUUGUAAACAGUUCC |
| 31 | 24181 | ACAUAUUCUUUAACUUCAA |
| 32 | 24182 | AAGCAGUCCUUUUACACUC |
| 33 | 24183 | UAGUGAGUUAUUUUGUCAA |
| 34 | 24184 | AGGAAGACAUCUUUGAACA |
| 35 | 24185 | GCAGUCCUUUUACACUCAA |
| 36 | 24186 | AGUUAUUUUGUCAAUAUAU |
| 37 | 24187 | GUACAACAGAAUAUGGUAU |
| 38 | 24188 | GUUAUUUUGUCAAUAUAUG |
| 39 | 24189 | CAGGCUUCAGGAAAAGAGG |
| 40 | 24190 | AGGAAAAGAGAUAAUUCCA |
| 41 | 24191 | UGUUACAGCAAUAUAAAGG |
| 42 | 24192 | UAUAAGCAUAUGCAAUCUC |
| 43 | 24193 | CAUAUUCUUUAACUUCAAA |
| 44 | 24194 | AAGACAUCUUUGAACACCU |
| 45 | 24195 | CCAGGAAGACAUCUUUGAA |
| 46 | 24196 | UACAGCAAUAUAAAGGUAC |
| 47 | 24197 | CAUUGUCAUAGGUUAUUGG |
| 48 | 24198 | UGAGUUAUUUUGUCAAUAU |
| 49 | 24199 | AGUGAGUUAUUUUGUCAAU |
| 50 | 24200 | GUGAGUUAUUUUGUCAAUA |
| 51 | 24201 | GAAUUUUCCUUGAAAGAUC |
| 52 | 24202 | ACUGUUACAGCAAUAUAAA |
| 53 | 24203 | AAAUCCAUUGUCAUAGGUU |
| 54 | 24204 | AAUCCAUUGUCAUAGGUUA |
| 55 | 24205 | GAGAAAUCCAUUGUCAUAG |
| 56 | 24206 | AAGACAUAUUCUUUAACUU |
| 57 | 24207 | AAGUGCAGAUUCCCUCCAC |
| 58 | 24208 | AUCCAUUGUCAUAGGUUAU |
| 59 | 24209 | AGACAUCUUUGAACACCUU |
| 60 | 24210 | CCAUUGUCAUAGGUUAUUG |
| 61 | 24211 | UGAAGAGAAAUCCAUUGUC |
| 62 | 24212 | GACAUAUUCUUUAACUUCA |
| 63 | 24213 | CAGUCCUUUUACACUCAAA |
| 64 | 24214 | AAUUUUCCUUGAAAGAUCC |
| 65 | 24215 | UGAAAUUGUAUUUUAUCUG |
| 66 | 24216 | AGUAAUUUCAAAAUUCUUA |
| 67 | 24217 | CAAAAUUCUUAAAGUUCUU |
| 68 | 24218 | UGAAUUUUGGUUCUGCUCU |
| 69 | 24219 | UGUCAUUUUAUAAUUAUGU |
| 70 | 24220 | CCAAAUCCUGUACUGACAA |
| 71 | 24221 | GGAUAACUUUUAAUAGAGA |
| 72 | 24222 | UUUAAGUCUUCUCUUAUUC |
| 73 | 24223 | GAUAAUUCCAAUAUGAUCA |
| 74 | 24224 | GGAUAAAUGAACAUGGCCU |
| 75 | 24225 | CAAGGUUCAUCAUUUUCUU |
| 76 | 24226 | UGGAAGUGCUAUAAAACAU |
| 77 | 24227 | CCAAGUACUCUUAAAGCAA |
| 78 | 24228 | UCCAAUGAUUUCCUGUUUC |
| 79 | 24229 | UAUGGUAUAUUCAUUUCCA |
| 80 | 24230 | GAACAAGAUGAACUUCCCA |
| 81 | 24231 | UGAACUUCAGGAAUUUUAG |
| 82 | 24232 | AAGUCUUCUCUUAUUCCAA |
| 83 | 24233 | GAAUGUUUAUACUUUGAUA |
| 84 | 24234 | CCGGAAUCGUACACAAAGG |
| 85 | 24235 | CAUACCUCUGCUCUUCUGA |
| 86 | 24236 | GAUCAAUUUCUUCUACCAU |
| 87 | 24237 | CAACAUUGUGUUUUGCAUU |
| 88 | 24238 | UAACUUUAUAAGCAUAUGC |
| 89 | 24239 | CAGGAUAACUUUUAAUAGA |
| 90 | 24240 | UUUUAUUGGUUGAUACUGU |
| 91 | 24241 | UGCAACUGUUUUCUUCUGG |
| 92 | 24242 | UGCUUUGAUACAACUUCCA |
| 93 | 24243 | CCAAAGCUUCUCUCUUCAA |
| 94 | 24244 | GGGAACUCCUUUCGUCUGC |
| 95 | 24245 | UAUGACAGUUCUUUGACUG |
| 96 | 24246 | UUGCAGAAUAACAUGUCCA |
| 97 | 24247 | CAGAAGUCCUAUAGUUGUA |
| 98 | 24248 | GAUAACUUUUAAUAGAGAU |
| 99 | 24249 | ACUAAGAUUUCUUUUCCAA |
| 100 | 24250 | GAUAAAUGAACAUGGCCUG |
| 101 | 24251 | GAUGAACAUGUUGUGUCUC |
| 102 | 24252 | UGAUCAUCUUUUAAGUCUU |
| 103 | 24253 | GAGCAAUUCCAUUUAUCAA |
| 104 | 24254 | UGUGAAUUUUCCUUGAAAG |
| 105 | 24255 | UCAAAAUUCUUAAAGUUCU |
| 106 | 24256 | UAAACUCCAGCACCGUCAC |
| 107 | 24257 | UUGAUAUUGGAAGUGCUAU |
| 108 | 24258 | GUGCAUUCAGUGUUACUGG |
| 109 | 24259 | AAUGUUUAUACUUUGAUAA |
| 110 | 24260 | AAAUUGUAUUUUAUCUGGA |
| 111 | 24261 | UAAGUCUUCUCUUAUUCCA |
| 112 | 24262 | AGAAGUCCUAUAGUUGUAA |
| 113 | 24263 | CUUGCUUUUAUAGUAAUUU |
| 114 | 24264 | UCAACAUUGUGUUUUGCAU |
| 115 | 24265 | AGGCAGUUGUUUCUACCAU |
| 116 | 24266 | UAUGAUCAAUUUCUUCUAC |
| 117 | 24267 | GUAAUUUCAAAAUUCUUAA |
| 118 | 24268 | AUCAACAUUGUGUUUUGCA |
| 119 | 24269 | CAAAGUAUUCCCAAAAGGC |
| 120 | 24270 | AAAACAUGGUACACUGUUU |
| 121 | 24271 | ACACAGAACUGCAUCCCAG |
| 122 | 24272 | UCCAAGUACUCUUAAAGCA |
| 123 | 24273 | UGUAGUAUGACAGUUCUUU |
| 124 | 24274 | CAGAAUAGCUUUCCCUUUU |
| 125 | 24275 | GCUUUUAUAGUAAUUUCAA |
| 126 | 24276 | GAAGCUACUCCAUCAUCAA |
| 127 | 24277 | UGCCACUAAUUCUAAGUAA |
| 128 | 24278 | GUAGACUCUAUGACUGUUA |
| 129 | 24279 | CUAAGAUUUCUUUUCCAAA |
| 130 | 24280 | CAAUAUUUAACCAGACUGA |
| 131 | 24281 | UAGUAAUUUCAAAAUUCUU |
| 132 | 24282 | GGAAAAGAGAUAAUUCCAA |
| 133 | 24283 | AAAAUGCUUGACACGAUGA |
| 134 | 24284 | UAUAUUCAUUUCCAGGAAG |
| 135 | 24285 | UAAUAAAAGCAAGUGCCAC |
| 136 | 24286 | UACUAUGCGUUUGUAAUCA |
| 137 | 24287 | GAAUUGAAAUACUCUUUCC |
| 138 | 24288 | UCAGGAUAACUUUUAAUAG |
| 139 | 24289 | UACAACUUCCAAAUACACA |
| 140 | 24290 | AAUCAUUCUCUAAUAAAAG |
| 141 | 24291 | GAAUAGCUUUCCCUUUUGA |
| 142 | 24292 | GGAAAUUCUUGUCUGUCAU |
| 143 | 24293 | UUGAAUUUUGGUUCUGCUC |
| 144 | 24294 | AGUGAGCUUUACAAAUAAG |
| 145 | 24295 | AAACAUGGUACACUGUUUA |
| 146 | 24296 | UAGACUCUAUGACUGUUAC |
| 147 | 24297 | UUUUCUUGGGAGUCAUCUG |
| 148 | 24298 | GUAGGCAAGGAGAUGUCCA |
| 149 | 24299 | AAAGCCACCUCCAUACCUC |
| 150 | 24300 | UAAGAUUUCUUUUCCAAAC |
| 151 | 24301 | AAAGCUGCAAACUUCCUCA |
| 152 | 24302 | UCUAAUAAAAGCAAGUGCC |
| 153 | 24303 | CAAUUGUUUGUGCAUUCAG |
| 154 | 24304 | ACCAAAGCUUCUCUCUUCA |
| 155 | 24305 | GCAACUGUUUUCUUCUGGG |
| 156 | 24306 | AUGCUUUGAUACAACUUCC |
| 157 | 24307 | AAUUCAUCUAAAUUAGCUA |
| 158 | 24308 | UGUGACGAUGUAAUAGACC |
| 159 | 24309 | GGUACACUGUUUAUCUGGU |
| 160 | 24310 | GGUUGCCUUGUACUUGACA |
| 161 | 24311 | CCAAGGAAAUUCUUGUCUG |
| 162 | 24312 | UUAACCAGACUGAAUCAGA |
| 163 | 24313 | UAGCAGCUAUUUCUUCUAU |
| 164 | 24314 | CCAGUUUUGUAGAUAUCCA |
| 165 | 24315 | AAUGAUUUCCUGUUUCCAG |
| 166 | 24316 | AGACAAGAUCUCACCUACA |
| 167 | 24317 | GUGGAUUACCUUUAACCAA |
| 168 | 24318 | UGAUACUGUGAAUUUUCCU |
| 169 | 24319 | AAUACUGGGACAACGCUCA |
| 170 | 24320 | CAUAAUUAAGUACUGUCUU |
| 171 | 24321 | CAAACUUCCUCAGAGGUAC |
| 172 | 24322 | AAGGCCAUGUUAUUUCAGA |
| 173 | 24323 | UGCAAACUGUAUGCAGCUG |
| 174 | 24324 | AGAAUAGCUUUCCCUUUUG |
| 175 | 24325 | UACUGUGAAUUUUCCUUGA |
| 176 | 24326 | CCAACUUCAAAGAGUUCAA |
| 177 | 24327 | CUGAAUUUUGAUAUUGGAA |
| 178 | 24328 | AAAGGCUGUAAGAUAUAAG |
| 179 | 24329 | UUGCUUUUAUAGUAAUUUC |
| 180 | 24330 | GUCCAAGUACUCUUAAAGC |
| 181 | 24331 | ACACUAAUUCUGCUGUCUG |
| 182 | 24332 | CUGGCUUGCUUACUGGUAA |
| 183 | 24333 | GUUGCCUUGUACUUGACAA |
| 184 | 24334 | CAGCAUUUCUGUAGGACAU |
| 185 | 24335 | UUAUACUUUGAUAAGAUGC |
| 186 | 24336 | UUAAGUACUGUCUUCCUUU |
| 187 | 24337 | CUGCAAACUUCCUCAGAGG |
| 188 | 24338 | AAGACAGUUUCUCUUUUGG |
| 189 | 24339 | AUUCAGUUUGUAGGGAGAG |
| 190 | 24340 | UGGAAUGUUUAUACUUUGA |
| 191 | 24341 | UAACAGGGUCUUCAUGUGU |
| 192 | 24342 | AGAGAUUGUUGCAUCAAAU |
| 193 | 24343 | GAGUCAUCUGCAUUUGCAU |
| 194 | 24344 | GAAUGUUUAUAUUUAGCAG |
| 195 | 24345 | GGAACAAGAUGAACUUCCC |
| 196 | 24346 | GGUAUUUCUGCCUCUUCAG |
| 197 | 24347 | CAGAACUGCAUCCCAGAAG |
| 198 | 24348 | UUCCGGUGUCCAAUAACCU |
| 199 | 24349 | CAUCAAUUGUUUGUGCAUU |
| 200 | 24350 | AAUACUCUUUCCAAGGGCU |
| 201 | 24351 | UCAAAUGCUUCAGUGUAUC |
| 202 | 24352 | AAGCAAGUGCCACUAAUUC |
| 203 | 24353 | UUAAUAGAGAUUGUUGCAU |
| 204 | 24354 | AUAGAGAUUGUUGCAUCAA |
| 205 | 24355 | GAACUCCUUUCGUCUGCUA |
| 206 | 24356 | GUCAUUUUAUAAUUAUGUA |
| 207 | 24357 | AAGAAUUGAAAUACUCUUU |
| 208 | 24358 | AAAGCCUUUCUAAUUCCAA |
| 209 | 24359 | UCAAUAUUUAACCAGACUG |
| 210 | 24360 | UGGUAUAUUCAUUUCCAGG |
| 211 | 24361 | UGAAACAAUUGAACGAAAC |
| 212 | 24362 | AAUAUCUUGCUUUUAUAGU |
| 213 | 24363 | AGAGAUUGUCAGAUCCAAU |
| 214 | 24364 | AGCAAUUCCAUUUAUCAAC |
| 215 | 24365 | UUAUCAGGAUAACUUUUAA |
| 216 | 24366 | CGAAUUUCUGGCUUGCUUA |
| 217 | 24367 | UCAAGUUCAGACUGGUGAG |
| 218 | 24368 | AGAGGUACUGGUUUUGUGA |
| 219 | 24369 | GGUAUUUUCUUUCAAGCAA |
| 220 | 24370 | AGGUACUGGUUUUGUGAAC |
| 221 | 24371 | CAGAUUCCCUCCACAGCAG |
| 222 | 24372 | UAGUCAGCAUUUCUGUAGG |
| 223 | 24373 | AUUAGCACGGAGCUGGCUU |
| 224 | 24374 | UAAGAGACACAGUUUGGCC |
| 225 | 24375 | GAACAUGUUGUGUCUCUAG |
| 226 | 24376 | CAUAAGUGCAAACUGUAUG |
| 227 | 24377 | CAAAUUCAGUUUGUAGGGA |
| 228 | 24378 | ACAAGCAGUCCUUUUACAC |
| 229 | 24379 | AAGUAUUCCCAAAAGGCCC |
| 230 | 24380 | CAAUCACAGUAAAGGCUGU |
| 231 | 24381 | AAUAGCUUUCCCUUUUGAC |
| 232 | 24382 | UAUUGGUUGAUACUGUGAA |
| 233 | 24383 | CAGAAAUGGCCAAUGUAAA |
| 234 | 24384 | UUGUAAUCAGAGUUUCCGU |
| 235 | 24385 | ACUCAGGCUUUAAUGAUCA |
| 236 | 24386 | CAGCUAUUUCUUCUAUCUU |
| 237 | 24387 | AUAUCUUGCUUUUAUAGUA |
| 238 | 24388 | ACAAGGUUCAUCAUUUUCU |
| 239 | 24389 | AUUGUGUUUUGCAUUGCUG |
| 240 | 24390 | GCCACAAUAAAGAAUUACA |
| 241 | 24391 | GGUGGAUUACCUUUAACCA |
| 242 | 24392 | CUUCUCUCUUCAAAGCUGA |
| 243 | 24393 | UGGGAUGCUUCAAUAUCCU |
| 244 | 24394 | AAAUCUUCUAAACUGUAGU |
| 245 | 24395 | AAAUUCAGUUUGUAGGGAG |
| 246 | 24396 | CUGUCAUUUUAUAAUUAUG |
| 247 | 24397 | AAUGCUUUGAUACAACUUC |
| 248 | 24398 | UCCUCUUUAUAUUUAGCCU |
| 249 | 24399 | GCAAAUUCAUCUAAAUUAG |
| 250 | 24400 | UUCCAAAUACACAUAAGAA |

TABLE 1b
Nucleobase sequences of the sense strands of 250
construc

| SEQ ID NO | Sense ID | 14 mer Sense |
|---|---|---|
| 251 | 14151 | AGGCCAAACUGUGU |
| 252 | 14152 | GACUUCAAGAUUCC |
| 253 | 14153 | AGCUACAAGCCCAG |
| 254 | 14154 | CUAUAAUUACUUGA |
| 255 | 14155 | AGACAAACCUGUUU |
| 256 | 14156 | GUUAAAAUGUCUGC |
| 257 | 14157 | AACCAGCUCCAGGU |
| 258 | 14158 | UACUUGAUUUUAUC |
| 259 | 14159 | GGCCAAACUGUGUC |
| 260 | 14160 | ACUUCAAGAUUCCG |
| 261 | 14161 | GUGUUAAAAUGUCU |
| 262 | 14162 | UUUUCCUGACUUCA |
| 263 | 14163 | CAGGCCAAACUGUG |
| 264 | 14164 | AACUCACUAUAAUU |
| 265 | 14165 | ACAUCUGACUUGGA |
| 266 | 14166 | AUCUCUUUUCCUGA |
| 267 | 14167 | GAAUAUGUCUUGCC |
| 268 | 14168 | GGAAAUUGGCCUUC |
| 269 | 14169 | ACUAUAAUUACUUG |
| 270 | 14170 | UUACUUGAUUUUAU |
| 271 | 14171 | UAUAAUUACUUGAU |
| 272 | 14172 | CUCUGGAAAUUGGC |
| 273 | 14173 | UCACUAUAAUUACU |
| 274 | 14174 | AACAAGUACCUUUA |
| 275 | 14175 | CUGAAACAGCAGUC |
| 276 | 14176 | UCUGAAACAGCAGU |
| 277 | 14177 | AAGUACCUUUAUAU |
| 278 | 14178 | AGGAACUGUUUACA |
| 279 | 14179 | CAGCCAAAGUUACC |
| 280 | 14180 | UGUUUACAACUAUA |
| 281 | 14181 | GUUAAAGAAUAUGU |
| 282 | 14182 | UAAAAGGACUGCUU |
| 283 | 14183 | AAAAUAACUCACUA |
| 284 | 14184 | AAAGAUGUCUUCCU |
| 285 | 14185 | UGUAAAAGGACUGC |
| 286 | 14186 | UUGACAAAAUAACU |
| 287 | 14187 | AUAUUCUGUUGUAC |
| 288 | 14188 | AUUGACAAAAUAAC |
| 289 | 14189 | UUUCCUGAAGCCUG |
| 290 | 14190 | UUAUCUCUUUUCCU |
| 291 | 14191 | AUAUUGCUGUAACA |
| 292 | 14192 | UGCAUAUGCUUAUA |
| 293 | 14193 | AGUUAAAGAAUAUG |
| 294 | 14194 | UUCAAAGAUGUCUU |
| 295 | 14195 | AGAUGUCUUCCUGG |
| 296 | 14196 | UUUAUAUUGCUGUA |
| 297 | 14197 | AACCUAUGACAAUG |
| 298 | 14198 | GACAAAAUAACUCA |
| 299 | 14199 | CAAAAUAACUCACU |
| 300 | 14200 | ACAAAAUAACUCAC |
| 301 | 14201 | UUCAAGGAAAAUUC |
| 302 | 14202 | AUUGCUGUAACAGU |
| 303 | 14203 | AUGACAAUGGAUUU |
| 304 | 14204 | UAUGACAAUGGAUU |
| 305 | 14205 | ACAAUGGAUUUCUC |
| 306 | 14206 | AAAGAAUAUGUCUU |
| 307 | 14207 | GGGAAUCUGCACUU |
| 308 | 14208 | CUAUGACAAUGGAU |
| 309 | 14209 | GUUCAAAGAUGUCU |
| 310 | 14210 | ACCUAUGACAAUGG |
| 311 | 14211 | UGGAUUUCUCUUCA |
| 312 | 14212 | UUAAAGAAUAUGUC |
| 313 | 14213 | GUGUAAAAGGACUG |
| 314 | 14214 | UUUCAAGGAAAAUU |
| 315 | 14215 | AAAAUACAAUUUCA |
| 316 | 14216 | AUUUUGAAAUUACU |
| 317 | 14217 | CUUUAAGAAUUUUG |
| 318 | 14218 | AGAACCAAAAUUCA |
| 319 | 14219 | AUUAUAAAAUGACA |
| 320 | 14220 | AGUACAGGAUUUGG |
| 321 | 14221 | AUUAAAAGUUAUCC |
| 322 | 14222 | AGAGAAGACUUAAA |
| 323 | 14223 | AUAUUGGAAUUAUC |
| 324 | 14224 | AUGUUCAUUUAUCC |
| 325 | 14225 | AAUGAUGAACCUUG |
| 326 | 14226 | UUAUAGCACUUCCA |
| 327 | 14227 | UUAAGAGUACUUGG |
| 328 | 14228 | AGGAAAUCAUUGGA |
| 329 | 14229 | AUGAAUAUACCAUA |
| 330 | 14230 | AGUUCAUCUUGUUC |
| 331 | 14231 | AUUCCUGAAGUUCA |
| 332 | 14232 | AUAAGAGAAGACUU |
| 333 | 14233 | AAGUAUAAACAUUC |
| 334 | 14234 | GUGUACGAUUCCGG |
| 335 | 14235 | AGAGCAGAGGUAUG |
| 336 | 14236 | AGAAGAAAUUGAUC |
| 337 | 14237 | AAAACACAAUGUUG |
| 338 | 14238 | UGCUUAUAAAGUUA |
| 339 | 14239 | UAAAAGUUAUCCUG |
| 340 | 14240 | AUCAACCAAUAAAA |
| 341 | 14241 | AGAAAACAGUUGCA |
| 342 | 14242 | GUUGUAUCAAAGCA |
| 343 | 14243 | GAGAGAAGCUUUGG |
| 344 | 14244 | CGAAAGGAGUUCCC |
| 345 | 14245 | AAAGAACUGUCAUA |
| 346 | 14246 | AUGUUAUUCUGCAA |
| 347 | 14247 | CUAUAGGACUUCUG |
| 348 | 14248 | UAUUAAAAGUUAUC |
| 349 | 14249 | AAAGAAAUCUUAGU |
| 350 | 14250 | CAUGUUCAUUUAUC |
| 351 | 14251 | ACAACAUGUUCAUC |
| 352 | 14252 | UUAAAAGAUGAUCA |
| 353 | 14253 | AAAUGGAAUUGCUC |
| 354 | 14254 | AAGGAAAAUUCACA |
| 355 | 14255 | UUUAAGAAUUUUGA |
| 356 | 14256 | GGUGCUGGAGUUUA |
| 357 | 14257 | ACUUCCAAUAUCAA |
| 358 | 14258 | AACACUGAAUGCAC |
| 359 | 14259 | AAAGUAUAAACAUU |
| 360 | 14260 | AUAAAAUACAAUUU |
| 361 | 14261 | UAAGAGAAGACUUA |
| 362 | 14262 | ACUAUAGGACUUCU |
| 363 | 14263 | ACUAUAAAAGCAAG |
| 364 | 14264 | AAACACAAUGUUGA |
| 365 | 14265 | AGAAACAACUGCCU |
| 366 | 14266 | AGAAAUUGAUCAUA |
| 367 | 14267 | AAUUUUGAAAUUAC |
| 368 | 14268 | AACACAAUGUUGAU |
| 369 | 14269 | UUGGGAAUACUUUG |
| 370 | 14270 | GUGUACCAUGUUUU |
| 371 | 14271 | AUGCAGUUCUGUGU |
| 372 | 14272 | UAAGAGUACUUGGA |
| 373 | 14273 | ACUGUCAUACUACA |
| 374 | 14274 | GGAAAGCUAUUCUG |
| 375 | 14275 | AUUACUAUAAAAGC |
| 376 | 14276 | GAUGGAGUAGCUUC |
| 377 | 14277 | UAGAAUUAGUGGCA |
| 378 | 14278 | GUCAUAGAGUCUAC |
| 379 | 14279 | AAAAGAAAUCUUAG |
| 380 | 14280 | CUGGUUAAAUAUUG |
| 381 | 14281 | UUUUGAAAUUACUA |
| 382 | 14282 | AUUAUCUCUUUUCC |
| 383 | 14283 | GUGUCAAGCAUUUU |
| 384 | 14284 | UGGAAAUGAAUAUA |
| 385 | 14285 | ACUUGCUUUUAUUA |
| 386 | 14286 | ACAAACGCAUAGUA |
| 387 | 14287 | GAGUAUUUCAAUUC |
| 388 | 14288 | AAAAGUUAUCCUGA |
| 389 | 14289 | AUUUGGAAGUUGUA |
| 390 | 14290 | AUUAGAGAAUGAUU |
| 391 | 14291 | AGGGAAAGCUAUUC |
| 392 | 14292 | AGACAAGAAUUUCC |
| 393 | 14293 | GAACCAAAAUUCAA |
| 394 | 14294 | UUGUAAAGCUCACU |
| 395 | 14295 | AGUGUACCAUGUUU |
| 396 | 14296 | AGUCAUAGAGUCUA |
| 397 | 14297 | GACUCCCAAGAAAA |
| 398 | 14298 | AUCUCCUUGCCUAC |
| 399 | 14299 | AUGGAGGUGGCUUU |
| 400 | 14300 | GAAAAGAAAUCUUA |
| 401 | 14301 | AAGUUUGCAGCUUU |
| 402 | 14302 | UUGCUUUUAUUAGA |
| 403 | 14303 | UGCACAAACAAUUG |
| 404 | 14304 | AGAGAAGCUUUGGU |
| 405 | 14305 | AAGAAAACAGUUGC |
| 406 | 14306 | UUGUAUCAAAGCAU |
| 407 | 14307 | AAUUUAGAUGAAUU |
| 408 | 14308 | AUUACAUCGUCACA |
| 409 | 14309 | AUAAACAGUGUACC |
| 410 | 14310 | AGUACAAGGCAACC |
| 411 | 14311 | AAGAAUUUCCUUGG |
| 412 | 14312 | UUCAGUCUGGUUAA |
| 413 | 14313 | AGAAAUAGCUGCUA |
| 414 | 14314 | AUCUACAAAACUGG |
| 415 | 14315 | AACAGGAAAUCAUU |
| 416 | 14316 | GUGAGAUCUUGUCU |
| 417 | 14317 | UAAAGGUAAUCCAC |
| 418 | 14318 | AAUUCACAGUAUCA |
| 419 | 14319 | GUUGUCCCAGUAUU |
| 420 | 14320 | AGUACUUAAUUAUG |
| 421 | 14321 | UCUGAGGAAGUUUG |
| 422 | 14322 | AAUAACAUGGCCUU |
| 423 | 14323 | GCAUACAGUUUGCA |
| 424 | 14324 | GGGAAAGCUAUUCU |
| 425 | 14325 | GAAAAUUCACAGUA |
| 426 | 14326 | CUCUUUGAAGUUGG |
| 427 | 14327 | AUAUCAAAAUUCAG |
| 428 | 14328 | AUCUUACAGCCUUU |
| 429 | 14329 | UACUAUAAAAGCAA |
| 430 | 14330 | AAGAGUACUUGGAC |
| 431 | 14331 | AGCAGAAUUAGUGU |
| 432 | 14332 | AGUAAGCAAGCCAG |
| 433 | 14333 | AAGUACAAGGCAAC |
| 434 | 14334 | CUACAGAAAUGCUG |
| 435 | 14335 | UUAUCAAAGUAUAA |
| 436 | 14336 | AAGACAGUACUUAA |
| 437 | 14337 | GAGGAAGUUUGCAG |
| 438 | 14338 | AGAGAAACUGUCUU |
| 439 | 14339 | CCUACAAACUGAAU |
| 440 | 14340 | GUAUAAACAUUCCA |
| 441 | 14341 | UGAAGACCCUGUUA |
| 442 | 14342 | AUGCAACAAUCUCU |
| 443 | 14343 | AAUGCAGAUGACUC |
| 444 | 14344 | AAAUAUAAACAUUC |
| 445 | 14345 | GUUCAUCUUGUUCC |
| 446 | 14346 | GAGGCAGAAAUACC |
| 447 | 14347 | GGGAUGCAGUUCUG |
| 448 | 14348 | AUUGGACACCGGAA |
| 449 | 14349 | ACAAACAAUUGAUG |
| 450 | 14350 | UUGGAAAGAGUAUU |
| 451 | 14351 | ACUGAAGCAUUUGA |
| 452 | 14352 | AGUGGCACUUGCUU |
| 453 | 14353 | ACAAUCUCUAUUAA |
| 454 | 14354 | GCAACAAUCUCUAU |
| 455 | 14355 | GACGAAAGGAGUUC |
| 456 | 14356 | AAUUAUAAAAUGAC |
| 457 | 14357 | GUAUUUCAAUUCUU |
| 458 | 14358 | AUUAGAAAGGCUUU |
| 459 | 14359 | UGGUUAAAUAUUGA |
| 460 | 14360 | AAAUGAAUAUACCA |
| 461 | 14361 | GUUCAAUUGUUUCA |
| 462 | 14362 | AAAAGCAAGAUAUU |
| 463 | 14363 | AUCUGACAAUCUCU |
| 464 | 14364 | UAAAUGGAAUUGCU |
| 465 | 14365 | AGUUAUCCUGAUAA |
| 466 | 14366 | AAGCCAGAAAUUCG |
| 467 | 14367 | CAGUCUGAACUUGA |
| 468 | 14368 | AAACCAGUACCUCU |
| 469 | 14369 | UGAAAGAAAAUACC |
| 470 | 14370 | CAAAACCAGUACCU |
| 471 | 14371 | GUGGAGGGAAUCUG |
| 472 | 14372 | AGAAAUGCUGACUA |
| 473 | 14373 | AGCUCCGUGCUAAU |
| 474 | 14374 | AACUGUGUCUCUUA |
| 475 | 14375 | GACACAACAUGUUC |
| 476 | 14376 | AGUUUGCACUUAUG |
| 477 | 14377 | ACAAACUGAAUUUG |
| 478 | 14378 | AAAGGACUGCUUGU |
| 479 | 14379 | UUUUGGGAAUACUU |
| 480 | 14380 | CUUUACUGUGAUUG |
| 481 | 14381 | AAGGGAAAGCUAUU |
| 482 | 14382 | AGUAUCAACCAAUA |
| 483 | 14383 | AUUGGCCAUUUCUG |
| 484 | 14384 | AACUCUGAUUACAA |
| 485 | 14385 | AUUAAAGCCUGAGU |
| 486 | 14386 | AGAAGAAAUAGCUG |
| 487 | 14387 | UAAAAGCAAGAUAU |
| 488 | 14388 | AUGAUGAACCUUGU |
| 489 | 14389 | AUGCAAAACACAAU |
| 490 | 14390 | UUCUUUAUUGUGGC |
| 491 | 14391 | AAAGGUAAUCCACC |
| 492 | 14392 | UUUGAAGAGAGAAG |
| 493 | 14393 | AUUGAAGCAUCCCA |
| 494 | 14394 | AGUUUAGAAGAUUU |
| 495 | 14395 | UACAAACUGAAUUU |
| 496 | 14396 | UUAUAAAAUGACAG |
| 497 | 14397 | UGUAUCAAAGCAUU |
| 498 | 14398 | AAAUAUAAAGAGGA |
| 499 | 14399 | UUAGAUGAAUUUGC |
| 500 | 14400 | AUGUGUAUUUGGAA |

Table 2 below shows the nucleobase sequences of the 250 hairpin constructs of the disclosure as selected in accordance with the Examples. The nucleobase sequences are a direct fusion of the antisense sequences of Table 1a with the corresponding sense sequences of Table 1b.

TABLE 2
Nucleobase sequences of 250 constructs in which the sense and
the antisense sequences of Tables 1a and 1b are combined.

| SEQ ID NO: | Sense ID | Antisense ID | Sequence of 33 mer Hairpin |
|---|---|---|---|
| 501 | 14151 | 24151 | ACACAGUUUGGCCUGGAGAAGGCCAAACUGU |
| | | | GU |
| 502 | 14152 | 24152 | GGAAUCUUGAAGUCAGGAAGACUUCAAGAUU |
| | | | CC |
| 503 | 14153 | 24153 | CUGGGCUUGUAGCUGGCACAGCUACAAGCCC |
| | | | AG |
| 504 | 14154 | 24154 | UCAAGUAAUUAUAGUGAGUCUAUAAUUACUU |
| | | | GA |
| 505 | 14155 | 24155 | AAACAGGUUUGUCUGUAUGAGACAAACCUGU |
| | | | UU |
| 506 | 14156 | 24156 | GCAGACAUUUUAACACAGAGUUAAAAUGUCU |
| | | | GC |
| 507 | 14157 | 24157 | ACCUGGAGCUGGUUGCCACAACCAGCUCCAG |
| | | | GU |
| 508 | 14158 | 24158 | GAUAAAAUCAAGUAAUUAUUACUUGAUUUUAU |
| | | | C |
| 509 | 14159 | 24159 | GACACAGUUUGGCCUGGAGGGCCAAACUGUG |
| | | | UC |
| 510 | 14160 | 24160 | CGGAAUCUUGAAGUCAGGAACUUCAAGAUUC |
| | | | CG |
| 511 | 14161 | 24161 | AGACAUUUUAACACAGAACGUGUUAAAAUGUC |
| | | | U |
| 512 | 14162 | 24162 | UGAAGUCAGGAAAAGAGAUUUUUCCUGACUU |
| | | | CA |
| 513 | 14163 | 24163 | CACAGUUUGGCCUGGAGAACAGGCCAAACUG |
| | | | UG |
| 514 | 14164 | 24164 | AAUUAUAGUGAGUUAUUUUAACUCACUAUAAU |
| | | | U |
| 515 | 14165 | 24165 | UCCAAGUCAGAUGUCUCUUACAUCUGACUUG |
| | | | GA |
| 516 | 14166 | 24166 | UCAGGAAAAGAGAUAAUUCAUCUCUUUUCCU |
| | | | GA |
| 517 | 14167 | 24167 | GGCAAGACAUAUUCUUUAAGAAUAUGUCUUG |
| | | | CC |
| 518 | 14168 | 24168 | GAAGGCCAAUUUCCAGAGGGGAAAUUGGCCU |
| | | | UC |
| 519 | 14169 | 24169 | CAAGUAAUUAUAGUGAGUUACUAUAAUUACUU |
| | | | G |
| 520 | 14170 | 24170 | AUAAAAUCAAGUAAUUAUAUUACUUGAUUUUA |
| | | | U |
| 521 | 14171 | 24171 | AUCAAGUAAUUAUAGUGAGUAUAAUUACUUGA |
| | | | U |
| 522 | 14172 | 24172 | GCCAAUUUCCAGAGGAAGCCUCUGGAAAUUG |
| | | | GC |
| 523 | 14173 | 24173 | AGUAAUUAUAGUGAGUUAUUCACUAUAAUUAC |
| | | | U |
| 524 | 14174 | 24174 | UAAAGGUACUUGUUGUUUAAACAAGUACCUU |
| | | | UA |
| 525 | 14175 | 24175 | GACUGCUGUUUCAGAAUCACUGAAACAGCAG |
| | | | UC |
| 526 | 14176 | 24176 | ACUGCUGUUUCAGAAUCAAUCUGAAACAGCA |
| | | | GU |
| 527 | 14177 | 24177 | AUAUAAAGGUACUUGUUGUAAGUACCUUUAU |
| | | | AU |
| 528 | 14178 | 24178 | UGUAAACAGUUCCUUUCAAAGGAACUGUUUA |
| | | | CA |
| 529 | 14179 | 24179 | GGUAACUUUGGCUGAGAGACAGCCAAAGUUA |
| | | | CC |
| 530 | 14180 | 24180 | UAUAGUUGUAAACAGUUCCUGUUUACAACUA |
| | | | UA |
| 531 | 14181 | 24181 | ACAUAUUCUUUAACUUCAAGUUAAAGAAUAUG |
| | | | U |
| 532 | 14182 | 24182 | AAGCAGUCCUUUUACACUCUAAAAGGACUGC |
| | | | UU |
| 533 | 14183 | 24183 | UAGUGAGUUAUUUUGUCAAAAAAUAACUCACU |
| | | | A |
| 534 | 14184 | 24184 | AGGAAGACAUCUUUGAACAAAAGAUGUCUUC |
| | | | CU |
| 535 | 14185 | 24185 | GCAGUCCUUUUACACUCAAUGUAAAAGGACU |
| | | | GC |
| 536 | 14186 | 24186 | AGUUAUUUUGUCAAUAUAUUUGACAAAAUAAC |
| | | | U |
| 537 | 14187 | 24187 | GUACAACAGAAUAUGGUAUAUAUUCUGUUGU |
| | | | AC |
| 538 | 14188 | 24188 | GUUAUUUUGUCAAUAUAUGAUUGACAAAAUAA |
| | | | C |
| 539 | 14189 | 24189 | CAGGCUUCAGGAAAAGAGGUUUCCUGAAGCC |
| | | | UG |
| 540 | 14190 | 24190 | AGGAAAAGAGAUAAUUCCAUUAUCUCUUUUCC |
| | | | U |
| 541 | 14191 | 24191 | UGUUACAGCAAUAUAAAGGAUAUUGCUGUAA |
| | | | CA |
| 542 | 14192 | 24192 | UAUAAGCAUAUGCAAUCUCUGCAUAUGCUUA |
| | | | UA |
| 543 | 14193 | 24193 | CAUAUUCUUUAACUUCAAAAGUUAAAGAAUAU |
| | | | G |
| 544 | 14194 | 24194 | AAGACAUCUUUGAACACCUUUCAAAGAUGUCU |
| | | | U |
| 545 | 14195 | 24195 | CCAGGAAGACAUCUUUGAAAGAUGUCUUCCU |
| | | | GG |
| 546 | 14196 | 24196 | UACAGCAAUAUAAAGGUACUUUAUAUUGCUG |
| | | | UA |
| 547 | 14197 | 24197 | CAUUGUCAUAGGUUAUUGGAACCUAUGACAA |
| | | | UG |
| 548 | 14198 | 24198 | UGAGUUAUUUUGUCAAUAUGACAAAAUAACUC |
| | | | A |
| 549 | 14199 | 24199 | AGUGAGUUAUUUUGUCAAUCAAAAUAACUCAC |
| | | | U |
| 550 | 14200 | 24200 | GUGAGUUAUUUUGUCAAUAACAAAAUAACUCA |
| | | | C |
| 551 | 14201 | 24201 | GAAUUUUCCUUGAAAGAUCUUCAAGGAAAAUU |
| | | | C |
| 552 | 14202 | 24202 | ACUGUUACAGCAAUAUAAAAUUGCUGUAACAG |
| | | | U |
| 553 | 14203 | 24203 | AAAUCCAUUGUCAUAGGUUAUGACAAUGGAU |
| | | | UU |
| 554 | 14204 | 24204 | AAUCCAUUGUCAUAGGUUAUAUGACAAUGGA |
| | | | UU |
| 555 | 14205 | 24205 | GAGAAAUCCAUUGUCAUAGACAAUGGAUUUC |
| | | | UC |
| 556 | 14206 | 24206 | AAGACAUAUUCUUUAACUUAAAGAAUAUGUCU |
| | | | U |
| 557 | 14207 | 24207 | AAGUGCAGAUUCCCUCCACGGGAAUCUGCAC |
| | | | UU |
| 558 | 14208 | 24208 | AUCCAUUGUCAUAGGUUAUCUAUGACAAUGG |
| | | | AU |
| 559 | 14209 | 24209 | AGACAUCUUUGAACACCUUGUUCAAAGAUGU |
| | | | CU |
| 560 | 14210 | 24210 | CCAUUGUCAUAGGUUAUUGACCUAUGACAAU |
| | | | GG |
| 561 | 14211 | 24211 | UGAAGAGAAAUCCAUUGUCUGGAUUUCUCUU |
| | | | CA |
| 562 | 14212 | 24212 | GACAUAUUCUUUAACUUCAUUAAAGAAUAUGU |
| | | | C |
| 563 | 14213 | 24213 | CAGUCCUUUUACACUCAAAGUGUAAAAGGAC |
| | | | UG |
| 564 | 14214 | 24214 | AAUUUUCCUUGAAAGAUCCUUUCAAGGAAAAU |
| | | | U |
| 565 | 14215 | 24215 | UGAAAUUGUAUUUUAUCUGAAAAUACAAUUUC |
| | | | A |
| 566 | 14216 | 24216 | AGUAAUUUCAAAAUUCUUAAUUUUGAAAUUAC |
| | | | U |
| 567 | 14217 | 24217 | CAAAAUUCUUAAAGUUCUUCUUUAAGAAUUUU |
| | | | G |
| 568 | 14218 | 24218 | UGAAUUUUGGUUCUGCUCUAGAACCAAAAUU |
| | | | CA |
| 569 | 14219 | 24219 | UGUCAUUUUAUAAUUAUGUAUUAUAAAAUGAC |
| | | | A |
| 570 | 14220 | 24220 | CCAAAUCCUGUACUGACAAAGUACAGGAUUU |
| | | | GG |
| 571 | 14221 | 24221 | GGAUAACUUUUAAUAGAGAAUUAAAAGUUAUC |
| | | | C |
| 572 | 14222 | 24222 | UUUAAGUCUUCUCUUAUUCAGAGAAGACUUA |
| | | | AA |
| 573 | 14223 | 24223 | GAUAAUUCCAAUAUGAUCAAUAUUGGAAUUAU |
| | | | C |
| 574 | 14224 | 24224 | GGAUAAAUGAACAUGGCCUAUGUUCAUUUAU |
| | | | CC |
| 575 | 14225 | 24225 | CAAGGUUCAUCAUUUUCUUAAUGAUGAACCU |
| | | | UG |
| 576 | 14226 | 24226 | UGGAAGUGCUAUAAAACAUUUAUAGCACUUC |
| | | | CA |
| 577 | 14227 | 24227 | CCAAGUACUCUUAAAGCAAUUAAGAGUACUUG |
| | | | G |
| 578 | 14228 | 24228 | UCCAAUGAUUUCCUGUUUCAGGAAAUCAUUG |
| | | | GA |
| 579 | 14229 | 24229 | UAUGGUAUAUUCAUUUCCAAUGAAUAUACCAU |
| | | | A |
| 580 | 14230 | 24230 | GAACAAGAUGAACUUCCCAAGUUCAUCUUGU |
| | | | UC |
| 581 | 14231 | 24231 | UGAACUUCAGGAAUUUUAGAUUCCUGAAGUU |
| | | | CA |
| 582 | 14232 | 24232 | AAGUCUUCUCUUAUUCCAAAUAAGAGAAGACU |
| | | | U |
| 583 | 14233 | 24233 | GAAUGUUUAUACUUUGAUAAAGUAUAAACAUU |
| | | | C |
| 584 | 14234 | 24234 | CCGGAAUCGUACACAAAGGGUGUACGAUUCC |
| | | | GG |
| 585 | 14235 | 24235 | CAUACCUCUGCUCUUCUGAAGAGCAGAGGUA |
| | | | UG |
| 586 | 14236 | 24236 | GAUCAAUUUCUUCUACCAUAGAAGAAAUUGAU |
| | | | C |
| 587 | 14237 | 24237 | CAACAUUGUGUUUUGCAUUAAAACACAAUGUU |
| | | | G |
| 588 | 14238 | 24238 | UAACUUUAUAAGCAUAUGCUGCUUAUAAAGUU |
| | | | A |
| 589 | 14239 | 24239 | CAGGAUAACUUUUAAUAGAUAAAAGUUAUCCU |
| | | | G |
| 590 | 14240 | 24240 | UUUUAUUGGUUGAUACUGUAUCAACCAAUAAA |
| | | | A |
| 591 | 14241 | 24241 | UGCAACUGUUUUCUUCUGGAGAAAACAGUUG |
| | | | CA |
| 592 | 14242 | 24242 | UGCUUUGAUACAACUUCCAGUUGUAUCAAAG |
| | | | CA |
| 593 | 14243 | 24243 | CCAAAGCUUCUCUCUUCAAGAGAGAAGCUUU |
| | | | GG |
| 594 | 14244 | 24244 | GGGAACUCCUUUCGUCUGCCGAAAGGAGUUC |
| | | | CC |
| 595 | 14245 | 24245 | UAUGACAGUUCUUUGACUGAAAGAACUGUCA |
| | | | UA |
| 596 | 14246 | 24246 | UUGCAGAAUAACAUGUCCAAUGUUAUUCUGC |
| | | | AA |
| 597 | 14247 | 24247 | CAGAAGUCCUAUAGUUGUACUAUAGGACUUC |
| | | | UG |
| 598 | 14248 | 24248 | GAUAACUUUUAAUAGAGAUUAUUAAAAGUUAU |
| | | | C |
| 599 | 14249 | 24249 | ACUAAGAUUUCUUUUCCAAAAAGAAAUCUUAG |
| | | | U |
| 600 | 14250 | 24250 | GAUAAAUGAACAUGGCCUGCAUGUUCAUUUA |
| | | | UC |
| 601 | 14251 | 24251 | GAUGAACAUGUUGUGUCUCACAACAUGUUCA |
| | | | UC |
| 602 | 14252 | 24252 | UGAUCAUCUUUUAAGUCUUUUAAAAGAUGAU |
| | | | CA |
| 603 | 14253 | 24253 | GAGCAAUUCCAUUUAUCAAAAAUGGAAUUGCU |
| | | | C |
| 604 | 14254 | 24254 | UGUGAAUUUUCCUUGAAAGAAGGAAAAUUCA |
| | | | CA |
| 605 | 14255 | 24255 | UCAAAAUUCUUAAAGUUCUUUUAAGAAUUUUG |
| | | | A |
| 606 | 14256 | 24256 | UAAACUCCAGCACCGUCACGGUGCUGGAGUU |
| | | | UA |
| 607 | 14257 | 24257 | UUGAUAUUGGAAGUGCUAUACUUCCAAUAUC |
| | | | AA |
| 608 | 14258 | 24258 | GUGCAUUCAGUGUUACUGGAACACUGAAUGC |
| | | | AC |
| 609 | 14259 | 24259 | AAUGUUUAUACUUUGAUAAAAAGUAUAAACAU |
| | | | U |
| 610 | 14260 | 24260 | AAAUUGUAUUUUAUCUGGAAUAAAAUACAAUU |
| | | | U |
| 611 | 14261 | 24261 | UAAGUCUUCUCUUAUUCCAUAAGAGAAGACU |
| | | | UA |
| 612 | 14262 | 24262 | AGAAGUCCUAUAGUUGUAAACUAUAGGACUU |
| | | | CU |
| 613 | 14263 | 24263 | CUUGCUUUUAUAGUAAUUUACUAUAAAAGCAA |
| | | | G |
| 614 | 14264 | 24264 | UCAACAUUGUGUUUUGCAUAAACACAAUGUU |
| | | | GA |
| 615 | 14265 | 24265 | AGGCAGUUGUUUCUACCAUAGAAACAACUGC |
| | | | CU |
| 616 | 14266 | 24266 | UAUGAUCAAUUUCUUCUACAGAAAUUGAUCAU |
| | | | A |
| 617 | 14267 | 24267 | GUAAUUUCAAAAUUCUUAAAAUUUUGAAAUUA |
| | | | C |
| 618 | 14268 | 24268 | AUCAACAUUGUGUUUUGCAAACACAAUGUUG |
| | | | AU |
| 619 | 14269 | 24269 | CAAAGUAUUCCCAAAAGGCUUGGGAAUACUU |
| | | | UG |
| 620 | 14270 | 24270 | AAAACAUGGUACACUGUUUGUGUACCAUGUU |
| | | | UU |
| 621 | 14271 | 24271 | ACACAGAACUGCAUCCCAGAUGCAGUUCUGU |
| | | | GU |
| 622 | 14272 | 24272 | UCCAAGUACUCUUAAAGCAUAAGAGUACUUG |
| | | | GA |
| 623 | 14273 | 24273 | UGUAGUAUGACAGUUCUUUACUGUCAUACUA |
| | | | CA |
| 624 | 14274 | 24274 | CAGAAUAGCUUUCCCUUUUGGAAAGCUAUUC |
| | | | UG |
| 625 | 14275 | 24275 | GCUUUUAUAGUAAUUUCAAAUUACUAUAAAAG |
| | | | C |
| 626 | 14276 | 24276 | GAAGCUACUCCAUCAUCAAGAUGGAGUAGCU |
| | | | UC |
| 627 | 14277 | 24277 | UGCCACUAAUUCUAAGUAAUAGAAUUAGUGG |
| | | | CA |
| 628 | 14278 | 24278 | GUAGACUCUAUGACUGUUAGUCAUAGAGUCU |
| | | | AC |
| 629 | 14279 | 24279 | CUAAGAUUUCUUUUCCAAAAAAAGAAAUCUUA |
| | | | G |
| 630 | 14280 | 24280 | CAAUAUUUAACCAGACUGACUGGUUAAAUAUU |
| | | | G |
| 631 | 14281 | 24281 | UAGUAAUUUCAAAAUUCUUUUUUGAAAUUACU |
| | | | A |
| 632 | 14282 | 24282 | GGAAAAGAGAUAAUUCCAAAUUAUCUCUUUUC |
| | | | C |
| 633 | 14283 | 24283 | AAAAUGCUUGACACGAUGAGUGUCAAGCAUU |
| | | | UU |
| 634 | 14284 | 24284 | UAUAUUCAUUUCCAGGAAGUGGAAAUGAAUA |
| | | | UA |
| 635 | 14285 | 24285 | UAAUAAAAGCAAGUGCCACACUUGCUUUUAUU |
| | | | A |
| 636 | 14286 | 24286 | UACUAUGCGUUUGUAAUCAACAAACGCAUAG |
| | | | UA |
| 637 | 14287 | 24287 | GAAUUGAAAUACUCUUUCCGAGUAUUUCAAU |
| | | | UC |
| 638 | 14288 | 24288 | UCAGGAUAACUUUUAAUAGAAAAGUUAUCCUG |
| | | | A |
| 639 | 14289 | 24289 | UACAACUUCCAAAUACACAAUUUGGAAGUUGU |
| | | | A |
| 640 | 14290 | 24290 | AAUCAUUCUCUAAUAAAAGAUUAGAGAAUGAU |
| | | | U |
| 641 | 14291 | 24291 | GAAUAGCUUUCCCUUUUGAAGGGAAAGCUAU |
| | | | UC |
| 642 | 14292 | 24292 | GGAAAUUCUUGUCUGUCAUAGACAAGAAUUU |
| | | | CC |
| 643 | 14293 | 24293 | UUGAAUUUUGGUUCUGCUCGAACCAAAAUUC |
| | | | AA |
| 644 | 14294 | 24294 | AGUGAGCUUUACAAAUAAGUUGUAAAGCUCA |
| | | | CU |
| 645 | 14295 | 24295 | AAACAUGGUACACUGUUUAAGUGUACCAUGU |
| | | | UU |
| 646 | 14296 | 24296 | UAGACUCUAUGACUGUUACAGUCAUAGAGUC |
| | | | UA |
| 647 | 14297 | 24297 | UUUUCUUGGGAGUCAUCUGGACUCCCAAGAA |
| | | | AA |
| 648 | 14298 | 24298 | GUAGGCAAGGAGAUGUCCAAUCUCCUUGCCU |
| | | | AC |
| 649 | 14299 | 24299 | AAAGCCACCUCCAUACCUCAUGGAGGUGGCU |
| | | | UU |
| 650 | 14300 | 24300 | UAAGAUUUCUUUUCCAAACGAAAAGAAAUCUU |
| | | | A |
| 651 | 14301 | 24301 | AAAGCUGCAAACUUCCUCAAAGUUUGCAGCU |
| | | | UU |
| 652 | 14302 | 24302 | UCUAAUAAAAGCAAGUGCCUUGCUUUUAUUA |
| | | | GA |
| 653 | 14303 | 24303 | CAAUUGUUUGUGCAUUCAGUGCACAAACAAU |
| | | | UG |
| 654 | 14304 | 24304 | ACCAAAGCUUCUCUCUUCAAGAGAAGCUUUG |
| | | | GU |
| 655 | 14305 | 24305 | GCAACUGUUUUCUUCUGGGAAGAAAACAGUU |
| | | | GC |
| 656 | 14306 | 24306 | AUGCUUUGAUACAACUUCCUUGUAUCAAAGC |
| | | | AU |
| 657 | 14307 | 24307 | AAUUCAUCUAAAUUAGCUAAAUUUAGAUGAAU |
| | | | U |
| 658 | 14308 | 24308 | UGUGACGAUGUAAUAGACCAUUACAUCGUCA |
| | | | CA |
| 659 | 14309 | 24309 | GGUACACUGUUUAUCUGGUAUAAACAGUGUA |
| | | | CC |
| 660 | 14310 | 24310 | GGUUGCCUUGUACUUGACAAGUACAAGGCAA |
| | | | CC |
| 661 | 14311 | 24311 | CCAAGGAAAUUCUUGUCUGAAGAAUUUCCUU |
| | | | GG |
| 662 | 14312 | 24312 | UUAACCAGACUGAAUCAGAUUCAGUCUGGUU |
| | | | AA |
| 663 | 14313 | 24313 | UAGCAGCUAUUUCUUCUAUAGAAAUAGCUGC |
| | | | UA |
| 664 | 14314 | 24314 | CCAGUUUUGUAGAUAUCCAAUCUACAAAACUG |
| | | | G |
| 665 | 14315 | 24315 | AAUGAUUUCCUGUUUCCAGAACAGGAAAUCA |
| | | | UU |
| 666 | 14316 | 24316 | AGACAAGAUCUCACCUACAGUGAGAUCUUGU |
| | | | CU |
| 667 | 14317 | 24317 | GUGGAUUACCUUUAACCAAUAAAGGUAAUCCA |
| | | | C |
| 668 | 14318 | 24318 | UGAUACUGUGAAUUUUCCUAAUUCACAGUAU |
| | | | CA |
| 669 | 14319 | 24319 | AAUACUGGGACAACGCUCAGUUGUCCCAGUA |
| | | | UU |
| 670 | 14320 | 24320 | CAUAAUUAAGUACUGUCUUAGUACUUAAUUAU |
| | | | G |
| 671 | 14321 | 24321 | CAAACUUCCUCAGAGGUACUCUGAGGAAGUU |
| | | | UG |
| 672 | 14322 | 24322 | AAGGCCAUGUUAUUUCAGAAAUAACAUGGCC |
| | | | UU |
| 673 | 14323 | 24323 | UGCAAACUGUAUGCAGCUGGCAUACAGUUUG |
| | | | CA |
| 674 | 14324 | 24324 | AGAAUAGCUUUCCCUUUUGGGGAAAGCUAUU |
| | | | CU |
| 675 | 14325 | 24325 | UACUGUGAAUUUUCCUUGAGAAAAUUCACAG |
| | | | UA |
| 676 | 14326 | 24326 | CCAACUUCAAAGAGUUCAACUCUUUGAAGUU |
| | | | GG |
| 677 | 14327 | 24327 | CUGAAUUUUGAUAUUGGAAAUAUCAAAAUUCA |
| | | | G |
| 678 | 14328 | 24328 | AAAGGCUGUAAGAUAUAAGAUCUUACAGCCU |
| | | | UU |
| 679 | 14329 | 24329 | UUGCUUUUAUAGUAAUUUCUACUAUAAAAGCA |
| | | | A |
| 680 | 14330 | 24330 | GUCCAAGUACUCUUAAAGCAAGAGUACUUGG |
| | | | AC |
| 681 | 14331 | 24331 | ACACUAAUUCUGCUGUCUGAGCAGAAUUAGU |
| | | | GU |
| 682 | 14332 | 24332 | CUGGCUUGCUUACUGGUAAAGUAAGCAAGCC |
| | | | AG |
| 683 | 14333 | 24333 | GUUGCCUUGUACUUGACAAAAGUACAAGGCA |
| | | | AC |
| 684 | 14334 | 24334 | CAGCAUUUCUGUAGGACAUCUACAGAAAUGC |
| | | | UG |
| 685 | 14335 | 24335 | UUAUACUUUGAUAAGAUGCUUAUCAAAGUAUA |
| | | | A |
| 686 | 14336 | 24336 | UUAAGUACUGUCUUCCUUUAAGACAGUACUU |
| | | | AA |
| 687 | 14337 | 24337 | CUGCAAACUUCCUCAGAGGGAGGAAGUUUGC |
| | | | AG |
| 688 | 14338 | 24338 | AAGACAGUUUCUCUUUUGGAGAGAAACUGUC |
| | | | UU |
| 689 | 14339 | 24339 | AUUCAGUUUGUAGGGAGAGCCUACAAACUGA |
| | | | AU |
| 690 | 14340 | 24340 | UGGAAUGUUUAUACUUUGAGUAUAAACAUUC |
| | | | CA |
| 691 | 14341 | 24341 | UAACAGGGUCUUCAUGUGUUGAAGACCCUGU |
| | | | UA |
| 692 | 14342 | 24342 | AGAGAUUGUUGCAUCAAAUAUGCAACAAUCUC |
| | | | U |
| 693 | 14343 | 24343 | GAGUCAUCUGCAUUUGCAUAAUGCAGAUGAC |
| | | | UC |
| 694 | 14344 | 24344 | GAAUGUUUAUAUUUAGCAGAAAUAUAAACAUU |
| | | | C |
| 695 | 14345 | 24345 | GGAACAAGAUGAACUUCCCGUUCAUCUUGUU |
| | | | CC |
| 696 | 14346 | 24346 | GGUAUUUCUGCCUCUUCAGGAGGCAGAAAUA |
| | | | CC |
| 697 | 14347 | 24347 | CAGAACUGCAUCCCAGAAGGGGAUGCAGUUC |
| | | | UG |
| 698 | 14348 | 24348 | UUCCGGUGUCCAAUAACCUAUUGGACACCGG |
| | | | AA |
| 699 | 14349 | 24349 | CAUCAAUUGUUUGUGCAUUACAAACAAUUGAU |
| | | | G |
| 700 | 14350 | 24350 | AAUACUCUUUCCAAGGGCUUUGGAAAGAGUA |
| | | | UU |
| 701 | 14351 | 24351 | UCAAAUGCUUCAGUGUAUCACUGAAGCAUUU |
| | | | GA |
| 702 | 14352 | 24352 | AAGCAAGUGCCACUAAUUCAGUGGCACUUGC |
| | | | UU |
| 703 | 14353 | 24353 | UUAAUAGAGAUUGUUGCAUACAAUCUCUAUUA |
| | | | A |
| 704 | 14354 | 24354 | AUAGAGAUUGUUGCAUCAAGCAACAAUCUCUA |
| | | | U |
| 705 | 14355 | 24355 | GAACUCCUUUCGUCUGCUAGACGAAAGGAGU |
| | | | UC |
| 706 | 14356 | 24356 | GUCAUUUUAUAAUUAUGUAAAUUAUAAAAUGA |
| | | | C |
| 707 | 14357 | 24357 | AAGAAUUGAAAUACUCUUUGUAUUUCAAUUCU |
| | | | U |
| 708 | 14358 | 24358 | AAAGCCUUUCUAAUUCCAAAUUAGAAAGGCUU |
| | | | U |
| 709 | 14359 | 24359 | UCAAUAUUUAACCAGACUGUGGUUAAAUAUU |
| | | | GA |
| 710 | 14360 | 24360 | UGGUAUAUUCAUUUCCAGGAAAUGAAUAUAC |
| | | | CA |
| 711 | 14361 | 24361 | UGAAACAAUUGAACGAAACGUUCAAUUGUUUC |
| | | | A |
| 712 | 14362 | 24362 | AAUAUCUUGCUUUUAUAGUAAAAGCAAGAUAU |
| | | | U |
| 713 | 14363 | 24363 | AGAGAUUGUCAGAUCCAAUAUCUGACAAUCU |
| | | | CU |
| 714 | 14364 | 24364 | AGCAAUUCCAUUUAUCAACUAAAUGGAAUUGC |
| | | | U |
| 715 | 14365 | 24365 | UUAUCAGGAUAACUUUUAAAGUUAUCCUGAUA |
| | | | A |
| 716 | 14366 | 24366 | CGAAUUUCUGGCUUGCUUAAAGCCAGAAAUU |
| | | | CG |
| 717 | 14367 | 24367 | UCAAGUUCAGACUGGUGAGCAGUCUGAACUU |
| | | | GA |
| 718 | 14368 | 24368 | AGAGGUACUGGUUUUGUGAAAACCAGUACCU |
| | | | CU |
| 719 | 14369 | 24369 | GGUAUUUUCUUUCAAGCAAUGAAAGAAAAUAC |
| | | | C |
| 720 | 14370 | 24370 | AGGUACUGGUUUUGUGAACCAAAACCAGUAC |
| | | | CU |
| 721 | 14371 | 24371 | CAGAUUCCCUCCACAGCAGGUGGAGGGAAUC |
| | | | UG |
| 722 | 14372 | 24372 | UAGUCAGCAUUUCUGUAGGAGAAAUGCUGAC |
| | | | UA |
| 723 | 14373 | 24373 | AUUAGCACGGAGCUGGCUUAGCUCCGUGCUA |
| | | | AU |
| 724 | 14374 | 24374 | UAAGAGACACAGUUUGGCCAACUGUGUCUCU |
| | | | UA |
| 725 | 14375 | 24375 | GAACAUGUUGUGUCUCUAGGACACAACAUGU |
| | | | UC |
| 726 | 14376 | 24376 | CAUAAGUGCAAACUGUAUGAGUUUGCACUUA |
| | | | UG |
| 727 | 14377 | 24377 | CAAAUUCAGUUUGUAGGGAACAAACUGAAUU |
| | | | UG |
| 728 | 14378 | 24378 | ACAAGCAGUCCUUUUACACAAAGGACUGCUU |
| | | | GU |
| 729 | 14379 | 24379 | AAGUAUUCCCAAAAGGCCCUUUUGGGAAUAC |
| | | | UU |
| 730 | 14380 | 24380 | CAAUCACAGUAAAGGCUGUCUUUACUGUGAU |
| | | | UG |
| 731 | 14381 | 24381 | AAUAGCUUUCCCUUUUGACAAGGGAAAGCUA |
| | | | UU |
| 732 | 14382 | 24382 | UAUUGGUUGAUACUGUGAAAGUAUCAACCAA |
| | | | UA |
| 733 | 14383 | 24383 | CAGAAAUGGCCAAUGUAAAAUUGGCCAUUUC |
| | | | UG |
| 734 | 14384 | 24384 | UUGUAAUCAGAGUUUCCGUAACUCUGAUUAC |
| | | | AA |
| 735 | 14385 | 24385 | ACUCAGGCUUUAAUGAUCAAUUAAAGCCUGA |
| | | | GU |
| 736 | 14386 | 24386 | CAGCUAUUUCUUCUAUCUUAGAAGAAAUAGC |
| | | | UG |
| 737 | 14387 | 24387 | AUAUCUUGCUUUUAUAGUAUAAAAGCAAGAUA |
| | | | U |
| 738 | 14388 | 24388 | ACAAGGUUCAUCAUUUUCUAUGAUGAACCUU |
| | | | GU |
| 739 | 14389 | 24389 | AUUGUGUUUUGCAUUGCUGAUGCAAAACACA |
| | | | AU |
| 740 | 14390 | 24390 | GCCACAAUAAAGAAUUACAUUCUUUAUUGUG |
| | | | GC |
| 741 | 14391 | 24391 | GGUGGAUUACCUUUAACCAAAAGGUAAUCCA |
| | | | CC |
| 742 | 14392 | 24392 | CUUCUCUCUUCAAAGCUGAUUUGAAGAGAGA |
| | | | AG |
| 743 | 14393 | 24393 | UGGGAUGCUUCAAUAUCCUAUUGAAGCAUCC |
| | | | CA |
| 744 | 14394 | 24394 | AAAUCUUCUAAACUGUAGUAGUUUAGAAGAUU |
| | | | U |
| 745 | 14395 | 24395 | AAAUUCAGUUUGUAGGGAGUACAAACUGAAU |
| | | | UU |
| 746 | 14396 | 24396 | CUGUCAUUUUAUAAUUAUGUUAUAAAAUGACA |
| | | | G |
| 747 | 14397 | 24397 | AAUGCUUUGAUACAACUUCUGUAUCAAAGCAU |
| | | | U |
| 748 | 14398 | 24398 | UCCUCUUUAUAUUUAGCCUAAAUAUAAAGAGG |
| | | | A |
| 749 | 14399 | 24399 | GCAAAUUCAUCUAAAUUAGUUAGAUGAAUUUG |
| | | | C |
| 750 | 14400 | 24400 | UUCCAAAUACACAUAAGAAAUGUGUAUUUGGA |
| | | | A |

Tables 3a to c below show 100 antisense sequences, sense sequences and hairpins of the disclosure, respectively; with full modification information (modified sugars and, where applicable, modified phosphates).

TABLE 3a
Modified antisense constructs

| SEQ | Anti- | |
|---|---|---|
| ID | sense | |
| NO | ID | Modified 19 mer Antisense |
| 751 | 24151 | [5Phos][mU][Ps][fC][Ps][mA][fC][mA][fG][mU][fU][mU][fG][mG][fC][mC][fU][Ps][mG] |
| | | [Ps][fG][Ps][mA][Ps][fG][Ps][mA] |
| 752 | 24152 | [5Phos][mU][Ps][fG][Ps][mA][fA][mU][fC][mU][fU][mG][fA][mA][fG][mU][fC][Ps][mA] |
| | | [Ps][fG][Ps][mG][Ps][fA][Ps][mA] |
| 753 | 24153 | [5Phos][mU][Ps][fU][Ps][mG][fG][mG][fC][mU][fU][mG][fU][mA][fG][mC][fU][Ps][mG] |
| | | [Ps][fG][Ps][mC][Ps][fA][Ps][mC] |
| 754 | 24154 | [5Phos][mU][Ps][fC][Ps][mA][fA][mG][fU][mA][fA][mU][fU][mA][fU][mA][fG][Ps][mU] |
| | | [Ps][fG][Ps][mA][Ps][fG][Ps][mU] |
| 755 | 24155 | [5Phos][mU][Ps][fA][Ps][mA][fC][mA][fG][mG][fU][mU][fU][mG][fU][mC][fU][Ps][mG] |
| | | [Ps][fU][Ps][mA][Ps][fU][Ps][mG] |
| 756 | 24156 | [5Phos][mU][Ps][fC][Ps][mA][fG][mA][fC][mA][fU][mU][fU][mU][fA][mA][fC][Ps][mA] |
| | | [Ps][fC][Ps][mA][Ps][fG][Ps][mA] |
| 757 | 24157 | [5Phos][mU][Ps][fC][Ps][mC][fU][mG][fG][mA][fG][mC][fU][mG][fG][mU][fU][Ps][mG] |
| | | [Ps][fC][Ps][mC][Ps][fA][Ps][mC] |
| 758 | 24158 | [5Phos][mU][Ps][fA][Ps][mU][fA][mA][fA][mA][fU][mC][fA][mA][fG][mU][fA][Ps][mA] |
| | | [Ps][fU][Ps][mU][Ps][fA][Ps][mU] |
| 759 | 24159 | [5Phos][mU][Ps][fA][Ps][mC][fA][mC][fA][mG][fU][mU][fU][mG][fG][mC][fC][Ps][mU] |
| | | [Ps][fG][Ps][mG][Ps][fA][Ps][mG] |
| 760 | 24160 | [5Phos][mU][Ps][fG][Ps][mG][fA][mA][fU][mC][fU][mU][fG][mA][fA][mG][fU][Ps][mC] |
| | | [Ps][fA][Ps][mG][Ps][fG][Ps][mA] |
| 761 | 24161 | [5Phos][mU][Ps][fG][Ps][mA][fC][mA][fU][mU][fU][mU][fA][mA][fC][mA][fC][Ps][mA] |
| | | [Ps][fG][Ps][mA][Ps][fA][Ps][mC] |
| 762 | 24162 | [5Phos][mU][Ps][fG][Ps][mA][fA][mG][fU][mC][fA][mG][fG][mA][fA][mA][fA][Ps][mG] |
| | | [Ps][fA][Ps][mG][Ps][fA][Ps][mU] |
| 763 | 24163 | [5Phos][mU][Ps][fA][Ps][mC][fA][mG][fU][mU][fU][mG][fG][mC][fC][mU][fG][Ps][mG] |
| | | [Ps][fA][Ps][mG][Ps][fA][Ps][mA] |
| 764 | 24164 | [5Phos][mU][Ps][fA][Ps][mU][fU][mA][fU][mA][fG][mU][fG][mA][fG][mU][fU][Ps][mA] |
| | | [Ps][fU][Ps][mU][Ps][fU][Ps][mU] |
| 765 | 24165 | [5Phos][mU][Ps][fC][Ps][mC][fA][mA][fG][mU][fC][mA][fG][mA][fU][mG][fU][Ps][mC] |
| | | [Ps][fU][Ps][mC][Ps][fU][Ps][mU] |
| 766 | 24166 | [5Phos][mU][Ps][fC][Ps][mA][fG][mG][fA][mA][fA][mA][fG][mA][fG][mA][fU][Ps][mA] |
| | | [Ps][fA][Ps][mU][Ps][fU][Ps][mC] |
| 767 | 24167 | [5Phos][mU][Ps][fG][Ps][mC][fA][mA][fG][mA][fC][mA][fU][mA][fU][mU][fC][Ps][mU] |
| | | [Ps][fU][Ps][mU][Ps][fA][Ps][mA] |
| 768 | 24168 | [5Phos][mU][Ps][fA][Ps][mA][fG][mG][fC][mC][fA][mA][fU][mU][fU][mC][fC][Ps][mA] |
| | | [Ps][fG][Ps][mA][Ps][fG][Ps][mG] |
| 769 | 24169 | [5Phos][mU][Ps][fA][Ps][mA][fG][mU][fA][mA][fU][mU][fA][mU][fA][mG][fU][Ps][mG] |
| | | [Ps][fA][Ps][mG][Ps][fU][Ps][mU] |
| 770 | 24170 | [5Phos][mU][Ps][fU][Ps][mA][fA][mA][fA][mU][fC][mA][fA][mG][fU][mA][fA][Ps][mU] |
| | | [Ps][fU][Ps][mA][Ps][fU][Ps][mA] |
| 771 | 24171 | [5Phos][mU][Ps][fU][Ps][mC][fA][mA][fG][mU][fA][mA][fU][mU][fA][mU][fA][Ps][mG] |
| | | [Ps][fU][Ps][mG][Ps][fA][Ps][mG] |
| 772 | 24172 | [5Phos][mU][Ps][fC][Ps][mC][fA][mA][fU][mU][fU][mC][fC][mA][fG][mA][fG][Ps][mG] |
| | | [Ps][fA][Ps][mA][Ps][fG][Ps][mC] |
| 773 | 24173 | [5Phos][mU][Ps][fG][Ps][mU][fA][mA][fU][mU][fA][mU][fA][mG][fU][mG][fA][Ps][mG] |
| | | [Ps][fU][Ps][mU][Ps][fA][Ps][mU] |
| 774 | 24174 | [5Phos][mU][Ps][fA][Ps][mA][fA][mG][fG][mU][fA][mC][fU][mU][fG][mU][fU][Ps][mG] |
| | | [Ps][fU][Ps][mU][Ps][fU][Ps][mA] |
| 775 | 24175 | [5Phos][mU][Ps][fA][Ps][mC][fU][mG][fC][mU][fG][mU][fU][mU][fC][mA][fG][Ps][mA] |
| | | [Ps][fA][Ps][mU][Ps][fC][Ps][mA] |
| 776 | 24176 | [5Phos][mU][Ps][fC][Ps][mU][fG][mC][fU][mG][fU][mU][fU][mC][fA][mG][fA][Ps][mA] |
| | | [Ps][fU][Ps][mC][Ps][fA][Ps][mA] |
| 777 | 24177 | [5Phos][mU][Ps][fU][Ps][mA][fU][mA][fA][mA][fG][mG][fU][mA][fC][mU][fU][Ps][mG] |
| | | [Ps][fU][Ps][mU][Ps][fG][Ps][mU] |
| 778 | 24178 | [5Phos][mU][Ps][fG][Ps][mU][fA][mA][fA][mC][fA][mG][fU][mU][fC][mC][fU][Ps][mU] |
| | | [Ps][fU][Ps][mC][Ps][fA][Ps][mA] |
| 779 | 24179 | [5Phos][mU][Ps][fG][Ps][mU][fA][mA][fC][mU][fU][mU][fG][mG][fC][mU][fG][Ps][mA] |
| | | [Ps][fG][Ps][mA][Ps][fG][Ps][mA] |
| 780 | 24180 | [5Phos][mU][Ps][fA][Ps][mU][fA][mG][fU][mU][fG][mU][fA][mA][fA][mC][fA][Ps][mG] |
| | | [Ps][fU][Ps][mU][Ps][fC][Ps][mC] |
| 781 | 24181 | [5Phos][mU][Ps][fC][Ps][mA][fU][mA][fU][mU][fC][mU][fU][mU][fA][mA][fC][Ps][mU] |
| | | [Ps][fA][Ps][mC][Ps][fU][Ps][mC] |
| 782 | 24182 | [5Phos][mU][Ps][fA][Ps][mG][fC][mA][fG][mU][fC][mC][fU][mU][fU][mU][fA][Ps][mC] |
| | | [Ps][fA][Ps][mC][Ps][fU][Ps][mC] |
| 783 | 24183 | [5Phos][mU][Ps][fA][Ps][mG][fU][mG][fA][mG][fU][mU][fA][mU][fU][mU][fU][Ps][mG] |
| | | [Ps][fU][Ps][mC][Ps][fA][Ps][mA] |
| 784 | 24184 | [5Phos][mU][Ps][fG][Ps][mG][fA][mA][fG][mA][fC][mA][fU][mC][fU][mU][fU][Ps][mG] |
| | | [Ps][fA][Ps][mA][Ps][fC][Ps][mA] |
| 785 | 24185 | [5Phos][mU][Ps][fC][Ps][mA][fG][mU][fC][mC][fU][mU][fU][mU][fA][mC][fA][Ps][mC] |
| | | [Ps][fU][Ps][mC][Ps][fA][Ps][mA] |
| 786 | 24186 | [5Phos][mU][Ps][fG][Ps][mU][fU][mA][fU][mU][fU][mU][fG][mU][fC][mA][fA][Ps][mU] |
| | | [Ps][fA][Ps][mU][Ps][fA][Ps][mU] |
| 787 | 24187 | [5Phos][mU][Ps][fU][Ps][mA][fC][mA][fA][mC][fA][mG][fA][mA][fU][mA][fU][Ps][mG] |
| | | [Ps][fG][Ps][mU][Ps][fA][Ps][mU] |
| 788 | 24188 | [5Phos][mU][Ps][fU][Ps][mU][fA][mU][fU][mU][fU][mG][fU][mC][fA][mA][fU][Ps][mA] |
| | | [Ps][fU][Ps][mA][Ps][fU][Ps][mG] |
| 789 | 24189 | [5Phos][mU][Ps][fA][Ps][mG][fG][mC][fU][mU][fC][mA][fG][mG][fA][mA][fA][Ps][mA] |
| | | [Ps][fG][Ps][mA][Ps][fG][Ps][mG] |
| 790 | 24190 | [5Phos][mU][Ps][fG][Ps][mG][fA][mA][fA][mA][fG][mA][fG][mA][fU][mA][fA][Ps][mU] |
| | | [Ps][fU][Ps][mC][Ps][fC][Ps][mA] |
| 791 | 24191 | [5Phos][mU][Ps][fG][Ps][mU][fU][mA][fC][mA][fG][mC][fA][mA][fU][mA][fU][Ps][mA] |
| | | [Ps][fA][Ps][mA][Ps][fG][Ps][mG] |
| 792 | 24192 | [5Phos][mU][Ps][fA][Ps][mU][fA][mA][fG][mC][fA][mU][fA][mU][fG][mC][fA][Ps][mA] |
| | | [Ps][fU][Ps][mC][Ps][fU][Ps][mC] |
| 793 | 24193 | [5Phos][mU][Ps][fA][Ps][mU][fA][mU][fU][mC][fU][mU][fU][mA][fA][mC][fU][Ps][mU] |
| | | [Ps][fC][Ps][mA][Ps][fA][Ps][mA] |
| 794 | 24194 | [5Phos][mU][Ps][fA][Ps][mG][fA][mC][fA][mU][fC][mU][fU][mU][fG][mA][fA][Ps][mC] |
| | | [Ps][fA][Ps][mC][Ps][fC][Ps][mU] |
| 795 | 24195 | [5Phos][mU][Ps][fC][Ps][mA][fG][mG][fA][mA][fG][mA][fC][mA][fU][mC][fU][Ps][mU] |
| | | [Ps][fU][Ps][mG][Ps][fA][Ps][mA] |
| 796 | 24196 | [5Phos][mU][Ps][fA][Ps][mC][fA][mG][fC][mA][fA][mU][fA][mU][fA][mA][fA][Ps][mG] |
| | | [Ps][fG][Ps][mU][Ps][fA][Ps][mC] |
| 797 | 24197 | [5Phos][mU][Ps][fA][Ps][mU][fU][mG][fU][mC][fA][mU][fA][mG][fG][mU][fU][Ps][mA] |
| | | [Ps][fU][Ps][mU][Ps][fG][Ps][mG] |
| 798 | 24198 | [5Phos][mU][Ps][fG][Ps][mA][fG][mU][fU][mA][fU][mU][fU][mU][fG][mU][fC][Ps][mA] |
| | | [Ps][fA][Ps][mU][Ps][fA][Ps][mU] |
| 799 | 24199 | [5Phos][mU][Ps][fG][Ps][mU][fG][mA][fG][mU][fU][mA][fU][mU][fU][mU][fG][Ps][mU] |
| | | [Ps][fC][Ps][mA][Ps][fA][Ps][mU] |
| 800 | 24200 | [5Phos][mU][Ps][fU][Ps][mG][fA][mG][fU][mU][fA][mU][fU][mU][fU][mG][fU][Ps][mC] |
| | | [Ps][fA][Ps][mA][Ps][fU][Ps][mA] |
| 801 | 24201 | [5Phos][mU][Ps][fA][Ps][mA][fU][mU][fU][mU][fC][mC][fU][mU][fG][mA][fA][Ps][mA] |
| | | [Ps][fG][Ps][mA][Ps][fU][Ps][mC] |
| 802 | 24202 | [5Phos][mU][Ps][fC][Ps][mU][fG][mU][fU][mA][fC][mA][fG][mC][fA][mA][fU][Ps][mA] |
| | | [Ps][fU][Ps][mA][Ps][fA][Ps][mA] |
| 803 | 24203 | [5Phos][mU][Ps][fA][Ps][mA][fU][mC][fC][mA][fU][mU][fG][mU][fC][mA][fU][Ps][mA] |
| | | [Ps][fG][Ps][mG][Ps][fU][Ps][mU] |
| 804 | 24204 | [5Phos][mU][Ps][fA][Ps][mU][fC][mC][fA][mU][fU][mG][fU][mC][fA][mU][fA][Ps][mG] |
| | | [Ps][fG][Ps][mU][Ps][fU][Ps][mA] |
| 805 | 24205 | [5Phos][mU][Ps][fA][Ps][mG][fA][mA][fA][mU][fC][mC][fA][mU][fU][mG][fU][Ps][mC] |
| | | [Ps][fA][Ps][mU][Ps][fA][Ps][mG] |
| 806 | 24206 | [5Phos][mU][Ps][fA][Ps][mG][fA][mC][fA][mU][fA][mU][fU][mC][fU][mU][fU][Ps][mA] |
| | | [Ps][fA][Ps][mC][Ps][fU][Ps][mU] |
| 807 | 24207 | [5Phos][mU][Ps][fA][Ps][mG][fU][mG][fC][mA][fG][mA][fU][mU][fC][mC][fC][Ps][mU] |
| | | [Ps][fC][Ps][mC][Ps][fA][Ps][mC] |
| 808 | 24208 | [5Phos][mU][Ps][fU][Ps][mC][fC][mA][fU][mU][fG][mU][fC][mA][fU][mA][fG][Ps][mG] |
| | | [Ps][fU][Ps][mU][Ps][fA][Ps][mU] |
| 809 | 24209 | [5Phos][mU][Ps][fG][Ps][mA][fC][mA][fU][mC][fU][mU][fU][mG][fA][mA][fC][Ps][mA] |
| | | [Ps][fC][Ps][mC][Ps][fU][Ps][mU] |
| 810 | 24210 | [5Phos][mU][Ps][fC][Ps][mA][fU][mU][fG][mU][fC][mA][fU][mA][fG][mG][fU][Ps][mU] |
| | | [Ps][fA][Ps][mU][Ps][fU][Ps][mG] |
| 811 | 24211 | [5Phos][mU][Ps][fG][Ps][mA][fA][mG][fA][mG][fA][mA][fA][mU][fC][mC][fA][Ps][mU] |
| | | [Ps][fU][Ps][mG][Ps][fU][Ps][mC] |
| 812 | 24212 | [5Phos][mU][Ps][fA][Ps][mC][fA][mU][fA][mU][fU][mC][fU][mU][fU][mA][fA][Ps][mC] |
| | | [Ps][fU][Ps][mU][Ps][fC][Ps][mA] |
| 813 | 24213 | [5Phos][mU][Ps][fA][Ps][mG][fU][mC][fC][mU][fU][mU][fU][mA][fC][mA][fC][Ps][mU] |
| | | [Ps][fC][Ps][mA][Ps][fA][Ps][mA] |
| 814 | 24214 | [5Phos][mU][Ps][fA][Ps][mU][fU][mU][fU][mC][fC][mU][fU][mG][fA][mA][fA][Ps][mG] |
| | | [Ps][fA][Ps][mU][Ps][fC][Ps][mC] |
| 815 | 24215 | [5Phos][mU][Ps][fG][Ps][mA][fA][mA][fU][mU][fG][mU][fA][mU][fU][mU][fU][Ps][mA] |
| | | [Ps][fU][Ps][mC][Ps][fU][Ps][mG] |
| 816 | 24216 | [5Phos][mU][Ps][fG][Ps][mU][fA][mA][fU][mU][fU][mC][fA][mA][fA][mA][fU][Ps][mU] |
| | | [Ps][fC][Ps][mU][Ps][fU][Ps][mA] |
| 817 | 24217 | [5Phos][mU][Ps][fA][Ps][mA][fA][mA][fU][mU][fC][mU][fU][mA][fA][mA][fG][Ps][mU] |
| | | [Ps][fU][Ps][mC][Ps][fU][Ps][mU] |
| 818 | 24218 | [5Phos][mU][Ps][fG][Ps][mA][fA][mU][fU][mU][fU][mG][fG][mU][fU][mC][fU][Ps][mG] |
| | | [Ps][fC][Ps][mU][Ps][fC][Ps][mU] |
| 819 | 24219 | [5Phos][mU][Ps][fG][Ps][mU][fC][mA][fU][mU][fU][mU][fA][mU][fA][mA][fU][Ps][mU] |
| | | [Ps][fA][Ps][mU][Ps][fG][Ps][mU] |
| 820 | 24220 | [5Phos][mU][Ps][fC][Ps][mA][fA][mA][fU][mC][fC][mU][fG][mU][fA][mC][fU][Ps][mG] |
| | | [Ps][fA][Ps][mC][Ps][fA][Ps][mA] |
| 821 | 24221 | [5Phos][mU][Ps][fG][Ps][mA][fU][mA][fA][mC][fU][mU][fU][mU][fA][mA][fU][Ps][mA] |
| | | [Ps][fG][Ps][mA][Ps][fG][Ps][mA] |
| 822 | 24222 | [5Phos][mU][Ps][fU][Ps][mU][fA][mA][fG][mU][fC][mU][fU][mC][fU][mC][fU][Ps][mU] |
| | | [Ps][fA][Ps][mU][Ps][fU][Ps][mC] |
| 823 | 24223 | [5Phos][mU][Ps][fA][Ps][mU][fA][mA][fU][mU][fC][mC][fA][mA][fU][mA][fU][Ps][mG] |
| | | [Ps][fA][Ps][mU][Ps][fC][Ps][mA] |
| 824 | 24224 | [5Phos][mU][Ps][fG][Ps][mA][fU][mA][fA][mA][fU][mG][fA][mA][fC][mA][fU][Ps][mG] |
| | | [Ps][fG][Ps][mC][Ps][fC][Ps][mU] |
| 825 | 24225 | [5Phos][mU][Ps][fA][Ps][mA][fG][mG][fU][mU][fC][mA][fU][mC][fA][mU][fU][Ps][mU] |
| | | [Ps][fU][Ps][mC][Ps][fU][Ps][mU] |
| 826 | 24226 | [5Phos][mU][Ps][fG][Ps][mG][fA][mA][fG][mU][fG][mC][fU][mA][fU][mA][fA][Ps][mA] |
| | | [Ps][fA][Ps][mC][Ps][fA][Ps][mU] |
| 827 | 24227 | [5Phos][mU][Ps][fC][Ps][mA][fA][mG][fU][mA][fC][mU][fC][mU][fU][mA][fA][Ps][mA] |
| | | [Ps][fG][Ps][mC][Ps][fA][Ps][mA] |
| 828 | 24228 | [5Phos][mU][Ps][fC][Ps][mC][fA][mA][fU][mG][fA][mU][fU][mU][fC][mC][fU][Ps][mG] |
| | | [Ps][fU][Ps][mU][Ps][fU][Ps][mC] |
| 829 | 24229 | [5Phos][mU][Ps][fA][Ps][mU][fG][mG][fU][mA][fU][mA][fU][mU][fC][mA][fU][Ps][mU] |
| | | [Ps][fU][Ps][mC][Ps][fC][Ps][mA] |
| 830 | 24230 | [5Phos][mU][Ps][fA][Ps][mA][fC][mA][fA][mG][fA][mU][fG][mA][fA][mC][fU][Ps][mU] |
| | | [Ps][fC][Ps][mC][Ps][fC][Ps][mA] |
| 831 | 24231 | [5Phos][mU][Ps][fG][Ps][mA][fA][mC][fU][mU][fC][mA][fG][mG][fA][mA][fU][Ps][mU] |
| | | [Ps][fU][Ps][mU][Ps][fA][Ps][mG] |
| 832 | 24232 | [5Phos][mU][Ps][fA][Ps][mG][fU][mC][fU][mU][fC][mU][fC][mU][fU][mA][fU][Ps][mU] |
| | | [Ps][fC][Ps][mC][Ps][fA][Ps][mA] |
| 833 | 24233 | [5Phos][mU][Ps][fA][Ps][mA][fU][mG][fU][mU][fU][mA][fU][mA][fC][mU][fU][Ps][mU] |
| | | [Ps][fG][Ps][mA][Ps][fU][Ps][mA] |
| 834 | 24234 | [5Phos][mU][Ps][fC][Ps][mG][fG][mA][fA][mU][fC][mG][fU][mA][fC][mA][fC][Ps][mA] |
| | | [Ps][fA][Ps][mA][Ps][fG][Ps][mG] |
| 835 | 24235 | [5Phos][mU][Ps][fA][Ps][mU][fA][mC][fC][mU][fC][mU][fG][mC][fU][mC][fU][Ps][mU] |
| | | [Ps][fC][Ps][mU][Ps][fG][Ps][mA] |
| 836 | 24236 | [5Phos][mU][Ps][fA][Ps][mU][fC][mA][fA][mU][fU][mU][fC][mU][fU][mC][fU][Ps][mA] |
| | | [Ps][fC][Ps][mC][Ps][fA][Ps][mU] |
| 837 | 24237 | [5Phos][mU][Ps][fA][Ps][mA][fC][mA][fU][mU][fG][mU][fG][mU][fU][mU][fU][Ps][mG] |
| | | [Ps][fC][Ps][mA][Ps][fU][Ps][mU] |
| 838 | 24238 | [5Phos][mU][Ps][fA][Ps][mA][fC][mU][fU][mU][fA][mU][fA][mA][fG][mC][fA][Ps][mU] |
| | | [Ps][fA][Ps][mU][Ps][fG][Ps][mC] |
| 839 | 24239 | [5Phos][mU][Ps][fA][Ps][mG][fG][mA][fU][mA][fA][mC][fU][mU][fU][mU][fA][Ps][mA] |
| | | [Ps][fU][Ps][mA][Ps][fG][Ps][mA] |
| 840 | 24240 | [5Phos][mU][Ps][fU][Ps][mU][fU][mA][fU][mU][fG][mG][fU][mU][fG][mA][fU][Ps][mA] |
| | | [Ps][fC][Ps][mU][Ps][fG][Ps][mU] |
| 841 | 24241 | [5Phos][mU][Ps][fG][Ps][mC][fA][mA][fC][mU][fG][mU][fU][mU][fU][mC][fU][Ps][mU] |
| | | [Ps][fU][Ps][mU][Ps][fG][Ps][mG] |
| 842 | 24242 | [5Phos][mU][Ps][fG][Ps][mC][fU][mU][fU][mG][fA][mU][fA][mC][fA][mA][fC][Ps][mU] |
| | | [Ps][fU][Ps][mC][Ps][fC][Ps][mA] |
| 843 | 24243 | [5Phos][mU][Ps][fC][Ps][mA][fA][mA][fG][mC][fU][mU][fC][mU][fC][mU][fC][Ps][mU] |
| | | [Ps][fU][Ps][mC][Ps][fA][Ps][mA] |
| 844 | 24244 | [5Phos][mU][Ps][fG][Ps][mG][fA][mA][fC][mU][fC][mC][fU][mU][fU][mC][fG][Ps][mU] |
| | | [Ps][fC][Ps][mU][Ps][fG][Ps][mC] |
| 845 | 24245 | [5Phos][mU][Ps][fA][Ps][mU][fG][mA][fC][mA][fG][mU][fU][mC][fU][mU][fU][Ps][mG] |
| | | [Ps][fA][Ps][mC][Ps][fU][Ps][mG] |
| 846 | 24246 | [5Phos][mU][Ps][fU][Ps][mG][fC][mA][fG][mA][fA][mU][fA][mA][fC][mA][fU][Ps][mG] |
| | | [Ps][fU][Ps][mC][Ps][fC][Ps][mA] |
| 847 | 24247 | [5Phos][mU][Ps][fA][Ps][mG][fA][mA][fG][mU][fC][mC][fU][mA][fU][mA][fG][Ps][mU] |
| | | [Ps][fU][Ps][mG][Ps][fU][Ps][mA] |
| 848 | 24248 | [5Phos][mU][Ps][fA][Ps][mU][fA][mA][fC][mU][fU][mU][fU][mA][fA][mU][fA][Ps][mG] |
| | | [Ps][fA][Ps][mG][Ps][fA][Ps][mU] |
| 849 | 24249 | [5Phos][mU][Ps][fC][Ps][mU][fA][mA][fG][mA][fU][mU][fU][mC][fU][mU][fU][Ps][mU] |
| | | [Ps][fC][Ps][mC][Ps][fA][Ps][mA] |
| 850 | 24250 | [5Phos][mU][Ps][fA][Ps][mU][fA][mA][fA][mU][fG][mA][fA][mC][fA][mU][fG][Ps][mG] |
| | | [Ps][fC][Ps][mC][Ps][fU][Ps][mG] |

Note =
Each of the above constructs may or may not have a phosphate modification at the 5′ end group. Furthermore, and independently, each of the above constructs may or may not have a “3x GalNAc” coupled to the 3′ end group. Optional are constructs with a 3x GalNAc ligand. Particularly optional are constructs which in addition have a 5′ phosphate, even though this is not a strict requirement, given that in the absence thereof, mammalian cells will add such phosphate in case it is absent from the molecule as administered.

TABLE 3b
Modified sense constructs of the present disclosure

| SEQ | | |
|---|---|---|
| ID | Sense | |
| NO | ID | Modified 15 mer Antisense |
| 851 | 14151 | [fC][Ps][mA][Ps][fG][mG][fC][mC][fA][mA][fA][mC] |
| | | [fU][mG][fU][Ps][mG][Ps][fA][3XGalNAc] |
| 852 | 14152 | [fU][Ps][mG][Ps][fA][mC][fU][mU][fC][mA][fA][mG] |
| | | [fA][mU][fU][Ps][mC][Ps][fA][3XGalNAc] |
| 853 | 14153 | [fC][Ps][mA][Ps][fG][mC][fU][mA][fC][mA][fA][mG] |
| | | [fC][mC][fC][Ps][mA][Ps][fA][3XGalNAc] |
| 854 | 14154 | [fA][Ps][mC][Ps][fU][mA][fU][mA][fA][mU][fU][mA] |
| | | [fC][mU][fU][Ps][mG][Ps][fA][3XGalNAc] |
| 855 | 14155 | [fC][Ps][mA][Ps][fG][mA][fC][mA][fA][mA][fC][mC] |
| | | [fU][mG][fU][Ps][mU][Ps][fA][3XGalNAc] |
| 856 | 14156 | [fU][Ps][mG][Ps][fU][mU][fA][mA][fA][mA][fU][mG] |
| | | [fU][mC][fU][Ps][mG][Ps][fA][3XGalNAc] |
| 857 | 14157 | [fC][Ps][mA][Ps][fA][mC][fC][mA][fG][mC][fU][mC] |
| | | [fC][mA][fG][Ps][mG][Ps][fA][3XGalNAc] |
| 858 | 14158 | [fU][Ps][mU][Ps][fA][mC][fU][mU][fG][mA][fU][mU] |
| | | [fU][mU][fA][Ps][mU][Ps][fA][3XGalNAc] |
| 859 | 14159 | [fA][Ps][mG][Ps][fG][mC][fC][mA][fA][mA][fC][mU] |
| | | [fG][mU][fG][Ps][mU][Ps][fA][3XGalNAc] |
| 860 | 14160 | [fG][Ps][mA][Ps][fC][mU][fU][mC][fA][mA][fG][mA] |
| | | [fU][mU][fC][Ps][mC][Ps][fA][3XGalNAc] |
| 861 | 14161 | [fU][Ps][mG][Ps][fU][mG][fU][mU][fA][mA][fA][mA] |
| | | [fU][mG][fU][Ps][mC][Ps][fA][3XGalNAc] |
| 862 | 14162 | [fC][Ps][mU][Ps][fU][mU][fU][mC][fC][mU][fG][mA] |
| | | [fC][mU][fU][Ps][mC][Ps][fA][3XGalNAc] |
| 863 | 14163 | [fC][Ps][mC][Ps][fA][mG][fG][mC][fC][mA][fA][mA] |
| | | [fC][mU][fG][Ps][mU][Ps][fA][3XGalNAc] |
| 864 | 14164 | [fU][Ps][mA][Ps][fA][mC][fU][mC][fA][mC][fU][mA] |
| | | [fU][mA][fA][Ps][mU][Ps][fA][3XGalNAc] |
| 865 | 14165 | [fG][Ps][mA][Ps][fC][mA][fU][mC][fU][mG][fA][mC] |
| | | [fU][mU][fG][Ps][mG][Ps][fA][3XGalNAc] |
| 866 | 14166 | [fU][Ps][mA][Ps][fU][mC][fU][mC][fU][mU][fU][mU] |
| | | [fC][mC][fU][Ps][mG][Ps][fA][3XGalNAc] |
| 867 | 14167 | [fA][Ps][mG][Ps][fA][mA][fU][mA][fU][mG][fU][mC] |
| | | [fU][mU][fG][Ps][mC][Ps][fA][3XGalNAc] |
| 868 | 14168 | [fU][Ps][mG][Ps][fG][mA][fA][mA][fU][mU][fG][mG] |
| | | [fC][mC][fU][Ps][mU][Ps][fA][3XGalNAc] |
| 869 | 14169 | [fC][Ps][mA][Ps][fC][mU][fA][mU][fA][mA][fU][mU] |
| | | [fA][mC][fU][Ps][mU][Ps][fA][3XGalNAc] |
| 870 | 14170 | [fA][Ps][mU][Ps][fU][mA][fC][mU][fU][mG][fA][mU] |
| | | [fU][mU][fU][Ps][mA][Ps][fA][3XGalNAc] |
| 871 | 14171 | [fC][Ps][mU][Ps][fA][mU][fA][mA][fU][mU][fA][mC] |
| | | [fU][mU][fG][Ps][mA][Ps][fA][3XGalNAc] |
| 872 | 14172 | [fC][Ps][mC][Ps][fU][mC][fU][mG][fG][mA][fA][mA] |
| | | [fU][mU][fG][Ps][mG][Ps][fA][3XGalNAc] |
| 873 | 14173 | [fC][Ps][mU][Ps][fC][mA][fC][mU][fA][mU][fA][mA] |
| | | [fU][mU][fA][Ps][mC][Ps][fA][3XGalNAc] |
| 874 | 14174 | [fC][Ps][mA][Ps][fA][mC][fA][mA][fG][mU][fA][mC] |
| | | [fC][mU][fU][Ps][mU][Ps][fA][3XGalNAc] |
| 875 | 14175 | [fU][Ps][mC][Ps][fU][mG][fA][mA][fA][mC][fA][mG] |
| | | [fC][mA][fG][Ps][mU][Ps][fA][3XGalNAc] |
| 876 | 14176 | [fU][Ps][mU][Ps][fC][mU][fG][mA][fA][mA][fC][mA] |
| | | [fG][mC][fA][Ps][mG][Ps][fA][3XGalNAc] |
| 877 | 14177 | [fC][Ps][mA][Ps][fA][mG][fU][mA][fC][mC][fU][mU] |
| | | [fU][mA][fU][Ps][mA][Ps][fA][3XGalNAc] |
| 878 | 14178 | [fA][Ps][mA][Ps][fG][mG][fA][mA][fC][mU][fG][mU] |
| | | [fU][mU][fA][Ps][mC][Ps][fA][3XGalNAc] |
| 879 | 14179 | [fU][Ps][mC][Ps][fA][mG][fC][mC][fA][mA][fA][mG][ |
| | | fU][mU][fA][Ps][mC][Ps][fA][3XGalNAc] |
| 880 | 14180 | [fC][Ps][mU][Ps][fG][mU][fU][mU][fA][mC][fA][mA] |
| | | [fC][mU][fA][Ps][mU][Ps][fA][3XGalNAc] |
| 881 | 14181 | [fA][Ps][mG][Ps][fU][mU][fA][mA][fA][mG][fA][mA] |
| | | [fU][mA][fU][Ps][mG][Ps][fA][3XGalNAc] |
| 882 | 14182 | [fG][Ps][mU][Ps][fA][mA][fA][mA][fG][mG][fA][mC] |
| | | [fU][mG][fC][Ps][mU][Ps][fA][3XGalNAc] |
| 883 | 14183 | [fC][Ps][mA][Ps][fA][mA][fA][mU][fA][mA][fC][mU] |
| | | [fC][mA][fC][Ps][mU][Ps][fA][3XGalNAc] |
| 884 | 14184 | [fC][Ps][mA][Ps][fA][mA][fG][mA][fU][mG][fU][mC] |
| | | [fU][mU][fC][Ps][mC][Ps][fA][3XGalNAc] |
| 885 | 14185 | [fG][Ps][mU][Ps][fG][mU][fA][mA][fA][mA][fG][mG] |
| | | [fA][mC][fU][Ps][mG][Ps][fA][3XGalNAc] |
| 886 | 14186 | [fA][Ps][mU][Ps][fU][mG][fA][mC][fA][mA][fA][mA] |
| | | [fU][mA][fA][Ps][mC][Ps][fA][3XGalNAc] |
| 887 | 14187 | [fC][Ps][mA][Ps][fU][mA][fU][mU][fC][mU][fG][mU] |
| | | [fU][mG][fU][Ps][mA][Ps][fA][3XGalNAc] |
| 888 | 14188 | [fU][Ps][mA][Ps][fU][mU][fG][mA][fC][mA][fA][mA] |
| | | [fA][mU][fA][Ps][mA][Ps][fA][3XGalNAc] |
| 889 | 14189 | [fU][Ps][mU][Ps][fU][mU][fC][mC][fU][mG][fA][mA] |
| | | [fG][mC][fC][Ps][mU][Ps][fA][3XGalNAc] |
| 890 | 14190 | [fA][Ps][mU][Ps][fU][mA][fU][mC][fU][mC][fU][mU] |
| | | [fU][mU][fC][Ps][mC][Ps][fA][3XGalNAc] |
| 891 | 14191 | [fU][Ps][mA][Ps][fU][mA][fU][mU][fG][mC][fU][mG] |
| | | [fU][mA][fA][Ps][mC][Ps][fA][3XGalNAc] |
| 892 | 14192 | [fU][Ps][mU][Ps][fG][mC][fA][mU][fA][mU][fG][mC] |
| | | [fU][mU][fA][Ps][mU][Ps][fA][3XGalNAc] |
| 893 | 14193 | [fA][Ps][mA][Ps][fG][mU][fU][mA][fA][mA][fG][mA] |
| | | [fA][mU][fA][Ps][mU][Ps][fA][3XGalNAc] |
| 894 | 14194 | [fG][Ps][mU][Ps][fU][mC][fA][mA][fA][mG][fA][mU] |
| | | [fG][mU][fC][Ps][mU][Ps][fA][3XGalNAc] |
| 895 | 14195 | [fA][Ps][mA][Ps][fG][mA][fU][mG][fU][mC][fU][mU] |
| | | [fC][mC][fU][Ps][mG][Ps][fA][3XGalNAc] |
| 896 | 14196 | [fC][Ps][mU][Ps][fU][mU][fA][mU][fA][mU][fU][mG] |
| | | [fC][mU][fG][Ps][mU][Ps][fA][3XGalNAc] |
| 897 | 14197 | [fU][Ps][mA][Ps][fA][mC][fC][mU][fA][mU][fG][mA] |
| | | [fC][mA][fA][Ps][mU][Ps][fA][3XGalNAc] |
| 898 | 14198 | [fU][Ps][mG][Ps][fA][mC][fA][mA][fA][mA][fU][mA] |
| | | [fA][mC][fU][Ps][mC][Ps][fA][3XGalNAc] |
| 899 | 14199 | [fA][Ps][mC][Ps][fA][mA][fA][mA][fU][mA][fA][mC] |
| | | [fU][mC][fA][Ps][mC][Ps][fA][3XGalNAc] |
| 900 | 14200 | [fG][Ps][mA][Ps][fC][mA][fA][mA][fA][mU][fA][mA] |
| | | [fC][mU][fC][Ps][mA][Ps][fA][3XGalNAc] |
| 901 | 14201 | [fU][Ps][mU][Ps][fU][mC][fA][mA][fG][mG][fA][mA] |
| | | [fA][mA][fU][Ps][mU][Ps][fA][3XGalNAc] |
| 902 | 14202 | [fU][Ps][mA][Ps][fU][mU][fG][mC][fU][mG][fU][mA] |
| | | [fA][mC][fA][Ps][mG][Ps][fA][3XGalNAc] |
| 903 | 14203 | [fU][Ps][mA][Ps][fU][mG][fA][mC][fA][mA][fU][mG] |
| | | [fG][mA][fU][Ps][mU][Ps][fA][3XGalNAc] |
| 904 | 14204 | [fC][Ps][mU][Ps][fA][mU][fG][mA][fC][mA][fA][mU] |
| | | [fG][mG][fA][Ps][mU][Ps][fA][3XGalNAc] |
| 905 | 14205 | [fG][Ps][mA][Ps][fC][mA][fA][mU][fG][mG][fA][mU] |
| | | [fU][mU][fC][Ps][mU][Ps][fA][3XGalNAc] |
| 906 | 14206 | [fU][Ps][mA][Ps][fA][mA][fG][mA][fA][mU][fA][mU] |
| | | [fG][mU][fC][Ps][mU][Ps][fA][3XGalNAc] |
| 907 | 14207 | [fA][Ps][mG][Ps][fG][mG][fA][mA][fU][mC][fU][mG] |
| | | [fC][mA][fC][Ps][mU][Ps][fA][3XGalNAc] |
| 908 | 14208 | [fC][Ps][mC][Ps][fU][mA][fU][mG][fA][mC][fA][mA] |
| | | [fU][mG][fG][Ps][mA][Ps][fA][3XGalNAc] |
| 909 | 14209 | [fA][Ps][mG][Ps][fG][mG][fA][mA][fU][mC][fU][mG] |
| | | [fC][mA][fC][Ps][mU][Ps][fA][3XGalNAc] |
| 910 | 14210 | [fA][Ps][mA][Ps][fC][mC][fU][mA][fU][mG][fA][mC] |
| | | [fA][mA][fU][Ps][mG][Ps][fA][3XGalNAc] |
| 911 | 14211 | [fA][Ps][mU][Ps][fG][mG][fA][mU][fU][mU][fC][mU] |
| | | [fC][mU][fU][Ps][mC][Ps][fA][3XGalNAc] |
| 912 | 14212 | [fG][Ps][mU][Ps][fU][mA][fA][mA][fG][mA][fA][mU] |
| | | [fA][mU][fG][Ps][mU][Ps][fA][3XGalNAc] |
| 913 | 14213 | [fA][Ps][mG][Ps][fU][mG][fU][mA][fA][mA][fA][mG] |
| | | [fG][mA][fC][Ps][mU][Ps][fA][3XGalNAc] |
| 914 | 14214 | [fC][Ps][mU][Ps][fU][mU][fC][mA][fA][mG][fG][mA] |
| | | [fA][mA][fA][Ps][mU][Ps][fA][3XGalNAc] |
| 915 | 14215 | [fU][Ps][mA][Ps][fA][mA][fA][mU][fA][mC][fA][mA] |
| | | [fU][mU][fU][Ps][mC][Ps][fA][3XGalNAc] |
| 916 | 14216 | [fA][Ps][mA][Ps][fU][mU][fU][mU][fG][mA][fA][mA] |
| | | [fU][mU][fA][Ps][mC][Ps][fA][3XGalNAc] |
| 917 | 14217 | [fA][Ps][mC][Ps][fU][mU][fU][mA][fA][mG][fA][mA] |
| | | [fU][mU][fU][Ps][mU][Ps][fA][3XGalNAc] |
| 918 | 14218 | [fC][Ps][mA][Ps][fG][mA][fA][mC][fC][mA][fA][mA] |
| | | [fA][mU][fU][Ps][mC][Ps][fA][3XGalNAc] |
| 919 | 14219 | [fA][Ps][mA][Ps][fU][mU][fA][mU][fA][mA][fA][mA] |
| | | [fU][mG][fA][Ps][mC][Ps][fA][3XGalNAc] |
| 920 | 14220 | [fC][Ps][mA][Ps][fG][mU][fA][mC][fA][mG][fG][mA] |
| | | [fU][mU][fU][Ps][mG][Ps][fA][3XGalNAc] |
| 921 | 14221 | [fU][Ps][mA][Ps][fU][mU][fA][mA][fA][mA][fG][mU] |
| | | [fU][mA][fU][Ps][mC][Ps][fA][3XGalNAc] |
| 922 | 14222 | [fA][Ps][mA][Ps][fG][mA][fG][mA][fA][mG][fA][mC] |
| | | [fU][mU][fA][Ps][mA][Ps][fA][3XGalNAc] |
| 923 | 14223 | [fC][Ps][mA][Ps][fU][mA][fU][mU][fG][mG][fA][mA] |
| | | [fU][mU][fA][Ps][mU][Ps][fA][3XGalNAc] |
| 924 | 14224 | [fC][Ps][mA][Ps][fU][mG][fU][mU][fC][mA][fU][mU] |
| | | [fU][mA][fU][Ps][mC][Ps][fA][3XGalNAc] |
| 925 | 14225 | [fA][Ps][mA][Ps][fA][mU][fG][mA][fU][mG][fA][mA] |
| | | [fC][mC][fU][Ps][mU][Ps][fA][3XGalNAc] |
| 926 | 14226 | [fU][Ps][mU][Ps][fU][mA][fU][mA][fG][mC][fA][mC] |
| | | [fU][mU][fC][Ps][mC][Ps][fA][3XGalNAc] |
| 927 | 14227 | [fU][Ps][mU][Ps][fU][mA][fA][mG][fA][mG][fU][mA] |
| | | [fC][mU][fU][Ps][mG][Ps][fA][3XGalNAc] |
| 928 | 14228 | [fC][Ps][mA][Ps][fG][mG][fA][mA][fA][mU][fC][mA] |
| | | [fU][mU][fG][Ps][mG][Ps][fA][3XGalNAc] |
| 929 | 14229 | [fA][Ps][mA][Ps][fU][mG][fA][mA][fU][mA][fU][mA] |
| | | [fC][mC][fA][Ps][mU][Ps][fA][3XGalNAc] |
| 930 | 14230 | [fA][Ps][mA][Ps][fG][mU][fU][mC][fA][mU][fC][mU] |
| | | [fU][mG][fU][Ps][mU][Ps][fA][3XGalNAc] |
| 931 | 14231 | [fA][Ps][mA][Ps][fU][mU][fC][mC][fU][mG][fA][mA] |
| | | [fG][mU][fU][Ps][mC][Ps][fA][3XGalNAc] |
| 932 | 14232 | [fA][Ps][mA][Ps][fU][mA][fA][mG][fA][mG][fA][mA] |
| | | [fG][mA][fC][Ps][mU][Ps][fA][3XGalNAc] |
| 933 | 14233 | [fA][Ps][mA][Ps][fA][mG][fU][mA][fU][mA][fA][mA] |
| | | [fC][mA][fU][Ps][mU][Ps][fA][3XGalNAc] |
| 934 | 14234 | [fU][Ps][mG][Ps][fU][mG][fU][mA][fC][mG][fA][mU] |
| | | [fU][mC][fC][Ps][mG][Ps][fA][3XGalNAc] |
| 935 | 14235 | [fA][Ps][mA][Ps][fG][mA][fG][mC][fA][mG][fA][mG] |
| | | [fG][mU][fA][Ps][mU][Ps][fA][3XGalNAc] |
| 936 | 14236 | [fU][Ps][mA][Ps][fG][mA][fA][mG][fA][mA][fA][mU] |
| | | [fU][mG][fA][Ps][mU][Ps][fA][3XGalNAc] |
| 937 | 14237 | [fC][Ps][mA][Ps][fA][mA][fA][mC][fA][mC][fA][mA] |
| | | [fU][mG][fU][Ps][mU][Ps][fA][3XGalNAc] |
| 938 | 14238 | [fA][Ps][mU][Ps][fG][mC][fU][mU][fA][mU][fA][mA] |
| | | [fA][mG][fU][Ps][mU][Ps][fA][3XGalNAc] |
| 939 | 14239 | [fU][Ps][mU][Ps][fA][mA][fA][mA][fG][mU][fU][mA] |
| | | [fU][mC][fC][Ps][mU][Ps][fA][3XGalNAc] |
| 940 | 14240 | [fU][Ps][mA][Ps][fU][mC][fA][mA][fC][mC][fA][mA] |
| | | [fU][mA][fA][Ps][mA][Ps][fA][3XGalNAc] |
| 941 | 14241 | [fA][Ps][mA][Ps][fG][mA][fA][mA][fA][mC][fA][mG] |
| | | [fU][mU][fG][Ps][mC][Ps][fA][3XGalNAc] |
| 942 | 14242 | [fA][Ps][mG][Ps][fU][mU][fG][mU][fA][mU][fC][mA] |
| | | [fA][mA][fG][Ps][mC][Ps][fA][3XGalNAc] |
| 943 | 14243 | [fA][Ps][mG][Ps][fA][mG][fA][mG][fA][mA][fG][mC] |
| | | [fU][mU][fU][Ps][mG][Ps][fA][3XGalNAc] |
| 944 | 14244 | [fA][Ps][mC][Ps][fG][mA][fA][mA][fG][mG][fA][mG] |
| | | [fU][mU][fC][Ps][mC][Ps][fA][3XGalNAc] |
| 945 | 14245 | [fC][Ps][mA][Ps][fA][mA][fG][mA][fA][mC][fU][mG] |
| | | [fU][mC][fA][Ps][mU][Ps][fA][3XGalNAc] |
| 946 | 14246 | [fC][Ps][mA][Ps][fU][mG][fU][mU][fA][mU][fU][mC] |
| | | [fU][mG][fC][Ps][mA][Ps][fA][3XGalNAc] |
| 947 | 14247 | [fA][Ps][mC][Ps][fU][mA][fU][mA][fG][mG][fA][mC] |
| | | [fU][mU][fC][Ps][mU][Ps][fA][3XGalNAc] |
| 948 | 14248 | [fC][Ps][mU][Ps][fA][mU][fU][mA][fA][mA][fA][mG] |
| | | [fU][mU][fA][Ps][mU][Ps][fA][3XGalNAc] |
| 949 | 14249 | [fA][Ps][mA][Ps][fA][mA][fG][mA][fA][mA][fU][mC] |
| | | [fU][mU][fA][Ps][mG][Ps][fA][3XGalNAc] |
| 950 | 14250 | [fC][Ps][mC][Ps][fA][mU][fG][mU][fU][mC][fA][mU] |
| | | [fU][mU][fA][Ps][mU][Ps][fA][3XGalNAc] |

Note =
each of the above constructs may or may not have a “3x GalNAc” coupled to the 3′ end group. Optional are constructs with a 3x GalNAc ligand, in particular a toothbrush ligand as defined herein.

TABLE 3c
Modified hairpin constructs of the present disclosure

| | Anti- | | |
|---|---|---|---|
| | sense | Experi- | |
| SEQ | ID + | mental | |
| ID | Sense | denota- | |
| NO | ID | tion | Modified 33 mer Hairpin |
| 951 | 14151_ | C5-m-01 | [5Phos][mU][Ps][fC][Ps][mA][fC][mA][fG][mU][fU][mU][fG][mG][fC][m |
| | 24151 | | C][fU][Ps][mG][Ps][fG][Ps][mA][Ps][fG][Ps][mA][Ps][mA] |
| | | | [fG][mG][fC][mC][fA][mA][fA][mC][fU][mG][fU][Ps][mG][Ps][fA][3xGal |
| | | | Nac] |
| 952 | 14152_ | C5-m-02 | [5Phos][mU][Ps][fG][Ps][mA][fA][mU][fC][mU][fU][mG][fA][mA][fG][m |
| | 24152 | | U][fC][Ps][mA][Ps][fG][Ps][mG][Ps][fA][Ps][mA][Ps][mG] |
| | | | [fA][mC][fU][mU][fC][mA][fA][mG][fA][mU][fU][Ps][mC][Ps][fA][3xGal |
| | | | Nac] |
| 953 | 14153_ | C5-m-03 | [5Phos][mU][Ps][fU][Ps][mG][fG][mG][fC][mU][fU][mG][fU][mA][fG][ |
| | 24153 | | mC][fU][Ps][mG][Ps][fG][Ps][mC][Ps][fA][Ps][mC][Ps][mA] |
| | | | [fG][mC][fU][mA][fC][mA][fA][mG][fC][mC][fC][Ps][mA][Ps][fA][3xGal |
| | | | Nac] |
| 954 | 14154_ | C5-m-04 | [5Phos][mU][Ps][fC][Ps][mA][fA][mG][fU][mA][fA][mU][fU][mA][fU][m |
| | 24154 | | A][fG][Ps][mU][Ps][fG][Ps][mA][Ps][fG][Ps][mU][Ps][mC] |
| | | | [fU][mA][fU][mA][fA][mU][fU][mA][fC][mU][fU][Ps][mG][Ps][fA][3xGal |
| | | | Nac] |
| 955 | 14155_ | C5-m-05 | [5Phos][mU][Ps][fA][Ps][mA][fC][mA][fG][mG][fU][mU][fU][mG][fU][m |
| | 24155 | | C][fU][Ps][mG][Ps][fU][Ps][mA][Ps][fU][Ps][mG][Ps][mA] |
| | | | [fG][mA][fC][mA][fA][mA][fC][mC][fU][mG][fU][Ps][mU][Ps][fA][3xGal |
| | | | Nac] |
| 956 | 14156_ | C5-m-06 | [5Phos][mU][Ps][fC][Ps][mA][fG][mA][fC][mA][fU][mU][fU][mU][fA][m |
| | 24156 | | A][fC][Ps][mA][Ps][fC][Ps][mA][Ps][fG][Ps][mA][Ps][mG] |
| | | | [fU][mU][fA][mA][fA][mA][fU][mG][fU][mC][fU][Ps][mG][Ps][fA][3xGal |
| | | | Nac] |
| 957 | 14157_ | C5-m-07 | [5Phos][mU][Ps][fC][Ps][mC][fU][mG][fG][mA][fG][mC][fU][mG][fG][ |
| | 24157 | | mU][fU][Ps][mG][Ps][fC][Ps][mC][Ps][fA][Ps][mC][Ps][mA] |
| | | | [fA][mC][fC][mA][fG][mC][fU][mC][fC][mA][fG][Ps][mG][Ps][fA][3xGal |
| | | | Nac] |
| 958 | 14158_ | C5-m-08 | [5Phos][mU][Ps][fA][Ps][mU][fA][mA][fA][mA][fU][mC][fA][mA][fG][m |
| | 24158 | | U][fA][Ps][mA][Ps][fU][Ps][mU][Ps][fA][Ps][mU][Ps][mU] |
| | | | [fA][mC][fU][mU][fG][mA][fU][mU][fU][mU][fA][Ps][mU][Ps][fA][3xGal |
| | | | Nac] |
| 959 | 14159_ | C5-m-09 | [5Phos][mU][Ps][fA][Ps][mC][fA][mC][fA][mG][fU][mU][fU][mG][fG][m |
| | 24159 | | C][fC][Ps][mU][Ps][fG][Ps][mG][Ps][fA][Ps][mG][Ps][mG] |
| | | | [fG][mC][fC][mA][fA][mA][fC][mU][fG][mU][fG][Ps][mU][Ps][fA][3xGal |
| | | | Nac] |
| 960 | 14160_ | C5-m-10 | [5Phos][mU][Ps][fG][Ps][mG][fA][mA][fU][mC][fU][mU][fG][mA][fA][m |
| | 24160 | | G][fU][Ps][mC][Ps][fA][Ps][mG][Ps][fG][Ps][mA][Ps][mA] |
| | | | [fC][mU][fU][mC][fA][mA][fG][mA][fU][mU][fC][Ps][mC][Ps][fA][3xGal |
| | | | Nac] |
| 961 | 14161_ | C5-m-11 | [5Phos][mU][Ps][fG][Ps][mA][fC][mA][fU][mU][fU][mU][fA][mA][fC][m |
| | 24161 | | A][fC][Ps][mA][Ps][fG][Ps][mA][Ps][fA][Ps][mC][Ps][mG] |
| | | | [fU][mG][fU][mU][fA][mA][fA][mA][fU][mG][fU][Ps][mC][Ps][fA][3xGal |
| | | | Nac] |
| 962 | 14162_ | C5-m-12 | [5Phos][mU][Ps][fG][Ps][mA][fA][mG][fU][mC][fA][mG][fG][mA][fA][m |
| | 24162 | | A][fA][Ps][mG][Ps][fA][Ps][mG][Ps][fA][Ps][mU][Ps][mU] |
| | | | [fU][mU][fU][mC][fC][mU][fG][mA][fC][mU][fU][Ps][mC][Ps][fA][3xGal |
| | | | Nac] |
| 963 | 14163_ | C5-m-13 | [5Phos][mU][Ps][fA][Ps][mC][fA][mG][fU][mU][fU][mG][fG][mC][fC][m |
| | 24163 | | U][fG][Ps][mG][Ps][fA][Ps][mG][Ps][fA][Ps][mA][Ps][mC] |
| | | | [fA][mG][fG][mC][fC][mA][fA][mA][fC][mU][fG][Ps][mU][Ps][fA][3xGal |
| | | | Nac] |
| 964 | 14164_ | C5-m-14 | [5Phos][mU][Ps][fA][Ps][mU][fU][mA][fU][mA][fG][mU][fG][mA][fG][m |
| | 24164 | | U][fU][Ps][mA][Ps][fU][Ps][mU][Ps][fU][Ps][mU][Ps][mA] |
| | | | [fA][mC][fU][mC][fA][mC][fU][mA][fU][mA][fA][Ps][mU][Ps][fA][3xGal |
| | | | Nac] |
| 965 | 14165_ | C5-m-15 | [5Phos][mU][Ps][fC][Ps][mC][fA][mA][fG][mU][fC][mA][fG][mA][fU][m |
| | 24165 | | G][fU][Ps][mC][Ps][fU][Ps][mC][Ps][fU][Ps][mU][Ps][mA] |
| | | | [fC][mA][fU][mC][fU][mG][fA][mC][fU][mU][fG][Ps][mG][Ps][fA][3xGal |
| | | | Nac] |
| 966 | 14166_ | C5-m-16 | [5Phos][mU][Ps][fC][Ps][mA][fG][mG][fA][mA][fA][mA][fG][mA][fG][m |
| | 24166 | | A][fU][Ps][mA][Ps][fA][Ps][mU][Ps][fU][Ps][mC][Ps][mA] |
| | | | [fU][mC][fU][mC][fU][mU][fU][mU][fC][mC][fU][Ps][mG][Ps][fA][3xGal |
| | | | Nac] |
| 967 | 14167_ | C5-m-17 | [5Phos][mU][Ps][fG][Ps][mC][fA][mA][fG][mA][fC][mA][fU][mA][fU][m |
| | 24167 | | U][fC][Ps][mU][Ps][fU][Ps][mU][Ps][fA][Ps][mA][Ps][mG] |
| | | | [fA][mA][fU][mA][fU][mG][fU][mC][fU][mU][fG][Ps][mC][Ps][fA][3xGal |
| | | | Nac] |
| 968 | 14168_ | C5-m-18 | [5Phos][mU][Ps][fA][Ps][mA][fG][mG][fC][mC][fA][mA][fU][mU][fU][m |
| | 24168 | | C][fC][Ps][mA][Ps][fG][Ps][mA][Ps][fG][Ps][mG][Ps][mG] |
| | | | [fG][mA][fA][mA][fU][mU][fG][mG][fC][mC][fU][Ps][mU][Ps][fA][3xGal |
| | | | Nac] |
| 969 | 14169_ | C5-m-19 | [5Phos][mU][Ps][fA][Ps][mA][fG][mU][fA][mA][fU][mU][fA][mU][fA][m |
| | 24169 | | G][fU][Ps][mG][Ps][fA][Ps][mG][Ps][fU][Ps][mU][Ps][mA] |
| | | | [fC][mU][fA][mU][fA][mA][fU][mU][fA][mC][fU][Ps][mU][Ps][fA][3xGal |
| | | | Nac] |
| 970 | 14170_ | C5-m-20 | [5Phos][mU][Ps][fU][Ps][mA][fA][mA][fA][mU][fC][mA][fA][mG][fU][m |
| | 24170 | | A][fA][Ps][mU][Ps][fU][Ps][mA][Ps][fU][Ps][mA][Ps][mU] |
| | | | [fU][mA][fC][mU][fU][mG][fA][mU][fU][mU][fU][Ps][mA][Ps][fA][3xGal |
| | | | Nac] |
| 971 | 14171_ | C5-m-21 | [5Phos][mU][Ps][fU][Ps][mC][fA][mA][fG][mU][fA][mA][fU][mU][fA][m |
| | 24171 | | U][fA][Ps][mG][Ps][fU][Ps][mG][Ps][fA][Ps][mG][Ps][mU] |
| | | | [fA][mU][fA][mA][fU][mU][fA][mC][fU][mU][fG][Ps][mA][Ps][fA][3xGal |
| | | | Nac] |
| 972 | 14172_ | C5-m-22 | [5Phos][mU][Ps][fC][Ps][mC][fA][mA][fU][mU][fU][mC][fC][mA][fG][m |
| | 24172 | | A][fG][Ps][mG][Ps][fA][Ps][mA][Ps][fG][Ps][mC][Ps][mC] |
| | | | [fU][mC][fU][mG][fG][mA][fA][mA][fU][mU][fG][Ps][mG][Ps][fA][3xGal |
| | | | Nac] |
| 973 | 14173_ | C5-m-23 | [5Phos][mU][Ps][fG][Ps][mU][fA][mA][fU][mU][fA][mU][fA][mG][fU][m |
| | 24173 | | G][fA][Ps][mG][Ps][fU][Ps][mU][Ps][fA][Ps][mU][Ps][mU] |
| | | | [fC][mA][fC][mU][fA][mU][fA][mA][fU][mU][fA][Ps][mC][Ps][fA][3xGal |
| | | | Nac] |
| 974 | 14174_ | C5-m-24 | [5Phos][mU][Ps][fA][Ps][mA][fA][mG][fG][mU][fA][mC][fU][mU][fG][m |
| | 24174 | | U][fU][Ps][mG][Ps][fU][Ps][mU][Ps][fU][Ps][mA][Ps][mA] |
| | | | [fA][mC][fA][mA][fG][mU][fA][mC][fC][mU][fU][Ps][mU][Ps][fA][3xGal |
| | | | Nac] |
| 975 | 14175_ | C5-m-25 | [5Phos][mU][Ps][fA][Ps][mC][fU][mG][fC][mU][fG][mU][fU][mU][fC][m |
| | 24175 | | A][fG][Ps][mA][Ps][fA][Ps][mU][Ps][fC][Ps][mA][Ps][mC] |
| | | | [fU][mG][fA][mA][fA][mC][fA][mG][fC][mA][fG][Ps][mU][Ps][fA][3xGal |
| | | | Nac] |
| 976 | 14176_ | C5-m-26 | [5Phos][mU][Ps][fC][Ps][mU][fG][mC][fU][mG][fU][mU][fU][mC][fA][m |
| | 24176 | | G][fA][Ps][mA][Ps][fU][Ps][mC][Ps][fA][Ps][mA][Ps][mU] |
| | | | [fC][mU][fG][mA][fA][mA][fC][mA][fG][mC][fA][Ps][mG][Ps][fA][3xGal |
| | | | Nac] |
| 977 | 14177_ | C5-m-27 | [5Phos][mU][Ps][fU][Ps][mA][fU][mA][fA][mA][fG][mG][fU][mA][fC][m |
| | 24177 | | U][fU][Ps][mG][Ps][fU][Ps][mU][Ps][fG][Ps][mU][Ps][mA] |
| | | | [fA][mG][fU][mA][fC][mC][fU][mU][fU][mA][fU][Ps][mA][Ps][fA][3xGal |
| | | | Nac] |
| 978 | 14178_ | C5-m-28 | [5Phos][mU][Ps][fG][Ps][mU][fA][mA][fA][mC][fA][mG][fU][mU][fC][m |
| | 24178 | | C][fU][Ps][mU][Ps][fU][Ps][mC][Ps][fA][Ps][mA][Ps][mA] |
| | | | [fG][mG][fA][mA][fC][mU][fG][mU][fU][mU][fA][Ps][mC][Ps][fA][3xGal |
| | | | Nac] |
| 979 | 14179_ | C5-m-29 | [5Phos][mU][Ps][fG][Ps][mU][fA][mA][fC][mU][fU][mU][fG][mG][fC][m |
| | 24179 | | U][fG][Ps][mA][Ps][fG][Ps][mA][Ps][fG][Ps][mA][Ps][mC] |
| | | | [fA][mG][fC][mC][fA][mA][fA][mG][fU][mU][fA][Ps][mC][Ps][fA][3xGal |
| | | | Nac] |
| 980 | 14180_ | C5-m-30 | [5Phos][mU][Ps][fA][Ps][mU][fA][mG][fU][mU][fG][mU][fA][mA][fA][m |
| | 24180 | | C][fA][Ps][mG][Ps][fU][Ps][mU][Ps][fC][Ps][mC][Ps][mU] |
| | | | [fG][mU][fU][mU][fA][mC][fA][mA][fC][mU][fA][Ps][mU][Ps][fA][3xGal |
| | | | Nac] |
| 981 | 14181_ | C5-m-31 | [5Phos][mU][Ps][fC][Ps][mA][fU][mA][fU][mU][fC][mU][fU][mU][fA][m |
| | 24181 | | A][fC][Ps][mU][Ps][fU][Ps][mC][Ps][fA][Ps][mA][Ps][mG][fU][mU] |
| | | | [fA][mA][fA][mG][fA][mA][fU][mA][fU][Ps][mG][Ps][fA][3xGal |
| | | | Nac] |
| 982 | 14182_ | C5-m-32 | [5Phos][mU][Ps][fA][Ps][mG][fC][mA][fG][mU][fC][mC][fU][mU][fU][m |
| | 24182 | | U][fA][Ps][mC][Ps][fA][Ps][mC][Ps][fU][Ps][mC][Ps][mU] |
| | | | [fA][mA][fA][mA][fG][mG][fA][mC][fU][mG][fC][Ps][mU][Ps][fA][3xGal |
| | | | Nac] |
| 983 | 14183_ | C5-m-33 | [5Phos][mU][Ps][fA][Ps][mG][fU][mG][fA][mG][fU][mU][fA][mU][fU][m |
| | 24183 | | U][fU][Ps][mG][Ps][fU][Ps][mC][Ps][fA][Ps][mA][Ps][mA] |
| | | | [fA][mA][fA][mU][fA][mA][fC][mU][fC][mA][fC][Ps][mU][Ps][fA][3xGal |
| | | | Nac] |
| 984 | 14184_ | C5-m-34 | [5Phos][mU][Ps][fG][Ps][mG][fA][mA][fG][mA][fC][mA][fU][mC][fU][m |
| | 24184 | | U][fU][Ps][mG][Ps][fA][Ps][mA][Ps][fC][Ps][mA][Ps][mA] |
| | | | [fA][mA][fG][mA][fU][mG][fU][mC][fU][mU][fC][Ps][mC][Ps][fA][3xGal |
| | | | Nac] |
| 985 | 14185_ | C5-m-35 | [5Phos][mU][Ps][fC][Ps][mA][fG][mU][fC][mC][fU][mU][fU][mU][fA][m |
| | 24185 | | C][fA][Ps][mC][Ps][fU][Ps][mC][Ps][fA][Ps][mA][Ps][mU] |
| | | | [fG][mU][fA][mA][fA][mA][fG][mG][fA][mC][fU][Ps][mG][Ps][fA][3xGal |
| | | | Nac] |
| 986 | 14186_ | C5-m-36 | [5Phos][mU][Ps][fG][Ps][mU][fU][mA][fU][mU][fU][mU][fG][mU][fC][m |
| | 24186 | | A][fA][Ps][mU][Ps][fA][Ps][mU][Ps][fA][Ps][mU][Ps][mU] |
| | | | [fU][mG][fA][mC][fA][mA][fA][mA][fU][mA][fA][Ps][mC][Ps][fA][3xGal |
| | | | Nac] |
| 987 | 14187_ | C5-m-37 | [5Phos][mU][Ps][fU][Ps][mA][fC][mA][fA][mC][fA][mG][fA][mA][fU][m |
| | 24187 | | A][fU][Ps][mG][Ps][fG][Ps][mU][Ps][fA][Ps][mU][Ps][mA] |
| | | | [fU][mA][fU][mU][fC][mU][fG][mU][fU][mG][fU][Ps][mA][Ps][fA][3xGal |
| | | | Nac] |
| 988 | 14188_ | C5-m-38 | [5Phos][mU][Ps][fU][Ps][mU][fA][mU][fU][mU][fU][mG][fU][mC][fA][m |
| | 24188 | | A][fU][Ps][mA][Ps][fU][Ps][mA][Ps][fU][Ps][mG][Ps][mA] |
| | | | [fU][mU][fG][mA][fC][mA][fA][mA][fA][mU][fA][Ps][mA][Ps][fA][3xGal |
| | | | Nac] |
| 989 | 14189_ | C5-m-39 | [5Phos][mU][Ps][fA][Ps][mG][fG][mC][fU][mU][fC][mA][fG][mG][fA][m |
| | 24189 | | A][fA][Ps][mA][Ps][fG][Ps][mA][Ps][fG][Ps][mG][Ps][mU] |
| | | | [fU][mU][fC][mC][fU][mG][fA][mA][fG][mC][fC][Ps][mU][Ps][fA][3xGal |
| | | | Nac] |
| 990 | 14190_ | C5-m-40 | [5Phos][mU][Ps][fG][Ps][mG][fA][mA][fA][mA][fG][mA][fG][mA][fU][m |
| | 24190 | | A][fA][Ps][mU][Ps][fU][Ps][mC][Ps][fC][Ps][mA][Ps][mU] |
| | | | [fU][mA][fU][mC][fU][mC][fU][mU][fU][mU][fC][Ps][mC][Ps][fA][3xGal |
| | | | Nac] |
| 991 | 14191_ | C5-m-41 | [5Phos][mU][Ps][fG][Ps][mU][fU][mA][fC][mA][fG][mC][fA][mA][fU][m |
| | 24191 | | A][fU][Ps][mA][Ps][fA][Ps][mA][Ps][fG][Ps][mG][Ps][mA] |
| | | | [fU][mA][fU][mU][fG][mC][fU][mG][fU][mA][fA][Ps][mC][Ps][fA][3xGal |
| | | | Nac] |
| 992 | 14192_ | C5-m-42 | [5Phos][mU][Ps][fA][Ps][mU][fA][mA][fG][mC][fA][mU][fA][mU][fG][m |
| | 24192 | | C][fA][Ps][mA][Ps][fU][Ps][mC][Ps][fU][Ps][mC][Ps][mU] |
| | | | [fG][mC][fA][mU][fA][mU][fG][mC][fU][mU][fA][Ps][mU][Ps][fA][3xGal |
| | | | Nac] |
| 993 | 14193_ | C5-m-43 | [5Phos][mU][Ps][fA][Ps][mU][fA][mU][fU][mC][fU][mU][fU][mA][fA][m |
| | 24193 | | C][fU][Ps][mU][Ps][fC][Ps][mA][Ps][fA][Ps][mA][Ps][mA] |
| | | | [fG][mU][fU][mA][fA][mA][fG][mA][fA][mU][fA][Ps][mU][Ps][fA][3xGal |
| | | | Nac] |
| 994 | 14194_ | C5-m-44 | [5Phos][mU][Ps][fA][Ps][mG][fA][mC][fA][mU][fC][mU][fU][mU][fG][m |
| | 24194 | | A][fA][Ps][mC][Ps][fA][Ps][mC][Ps][fC][Ps][mU][Ps][mU] |
| | | | [fU][mC][fA][mA][fA][mG][fA][mU][fG][mU][fC][Ps][mU][Ps][fA][3xGal |
| | | | Nac] |
| 995 | 14195_ | C5-m-45 | [5Phos][mU][Ps][fC][Ps][mA][fG][mG][fA][mA][fG][mA][fC][mA][fU][m |
| | 24195 | | C][fU][Ps][mU][Ps][fU][Ps][mG][Ps][fA][Ps][mA][Ps][mA] |
| | | | [fG][mA][fU][mG][fU][mC][fU][mU][fC][mC][fU][Ps][mG][Ps][fA][3xGal |
| | | | Nac] |
| 996 | 14196_ | C5-m-46 | [5Phos][mU][Ps][fA][Ps][mC][fA][mG][fC][mA][fA][mU][fA][mU][fA][m |
| | 24196 | | A][fA][Ps][mG][Ps][fG][Ps][mU][Ps][fA][Ps][mC][Ps][mU] |
| | | | [fU][mU][fA][mU][fA][mU][fU][mG][fC][mU][fG][Ps][mU][Ps][fA][3xGal |
| | | | Nac] |
| 997 | 14197_ | C5-m-47 | [5Phos][mU][Ps][fA][Ps][mU][fU][mG][fU][mC][fA][mU][fA][mG][fG][m |
| | 24197 | | U][fU][Ps][mA][Ps][fU][Ps][mU][Ps][fG][Ps][mG][Ps][mA][fA][mC][fC |
| | | | ][mU][fA][mU][fG][mA][fC][mA][fA][Ps][mU][Ps][fA][3xGalNac] |
| 998 | 14198_ | C5-m-48 | [5Phos][mU][Ps][fG][Ps][mA][fG][mU][fU][mA][fU][mU][fU][mU][fG][m |
| | 24198 | | U][fC][Ps][mA][Ps][fA][Ps][mU][Ps][fA][Ps][mU][Ps][mG][fA][mC][fA |
| | | | ][mA][fA][mA][fU][mA][fA][mC][fU][Ps][mC][Ps][fA][3xGalNac] |
| 999 | 14199_ | C5-m-49 | [5Phos][mU][Ps][fG][Ps][mU][fG][mA][fG][mU][fU][mA][fU][mU][fU][m |
| | 24199 | | U][fG][Ps][mU][Ps][fC][Ps][mA][Ps][fA][Ps][mU][Ps][mC][fA][mA][fA |
| | | | ][mA][fU][mA][fA][mC][fU][mC][fA][Ps][mC][Ps][fA][3xGalNac] |
| 1000 | 14200_ | C5-m-50 | [5Phos][mU][Ps][fU][Ps][mG][fA][mG][fU][mU][fA][mU][fU][mU][fU][m |
| | 24200 | | G][fU][Ps][mC][Ps][fA][Ps][mA][Ps][fU][Ps][mA][Ps][mA][fC][mA][fA |
| | | | ][mA][fA][mU][fA][mA][fC][mU][fC][Ps][mA][Ps][fA][3xGalNac] |
| 1001 | 14201_ | C5-m-51 | [5Phos][mU][Ps][fA][Ps][mA][fU][mU][fU][mU][fC][mC][fU][mU][fG][m |
| | 24201 | | A][fA][Ps][mA][Ps][fG][Ps][mA][Ps][fU][Ps][mC][Ps][mU][fU][mC][fA |
| | | | ][mA][fG][mG][fA][mA][fA][mA][fU][Ps][mU][Ps][fA][3xGalNac] |
| 1002 | 14202_ | C5-m-52 | [5Phos][mU][Ps][fC][Ps][mU][fG][mU][fU][mA][fC][mA][fG][mC][fA][m |
| | 24202 | | A][fU][Ps][mA][Ps][fU][Ps][mA][Ps][fA][Ps][mA][Ps][mA][fU][mU][fG |
| | | | ][mC][fU][mG][fU][mA][fA][mC][fA][Ps][mG][Ps][fA][3xGalNac] |
| 1003 | 14203_ | C5-m-53 | [5Phos][mU][Ps][fA][Ps][mA][fU][mC][fC][mA][fU][mU][fG][mU][fC][m |
| | 24203 | | A][fU][Ps][mA][Ps][fG][Ps][mG][Ps][fU][Ps][mU][Ps][mA][fU][mG][fA |
| | | | ][mC][fA][mA][fU][mG][fG][mA][fU][Ps][mU][Ps][fA][3xGalNac] |
| 1004 | 14204_ | C5-m-54 | [5Phos][mU][Ps][fA][Ps][mU][fC][mC][fA][mU][fU][mG][fU][mC][fA][m |
| | 24204 | | U][fA][Ps][mG][Ps][fG][Ps][mU][Ps][fU][Ps][mA][Ps][mU][fA][mU][fG |
| | | | ][mA][fC][mA][fA][mU][fG][mG][fA][Ps][mU][Ps][fA][3xGalNac] |
| 1005 | 14205_ | C5-m-55 | [5Phos][mU][Ps][fA][Ps][mG][fA][mA][fA][mU][fC][mC][fA][mU][fU][m |
| | 24205 | | G][fU][Ps][mC][Ps][fA][Ps][mU][Ps][fA][Ps][mG][Ps][mA][fC][mA][fA |
| | | | ][mU][fG][mG][fA][mU][fU][mU][fC][Ps][mU][Ps][fA][3xGalNac] |
| 1006 | 14206_ | C5-m-56 | [5Phos][mU][Ps][fA][Ps][mG][fA][mC][fA][mU][fA][mU][fU][mC][fU][m |
| | 24206 | | U][fU][Ps][mA][Ps][fA][Ps][mC][Ps][fU][Ps][mU][Ps][mA][fA][mA][fG |
| | | | ][mA][fA][mU][fA][mU][fG][mU][fC][Ps][mU][Ps][fA][3xGalNac] |
| 1007 | 14207_ | C5-m-57 | [5Phos][mU][Ps][fA][Ps][mG][fU][mG][fC][mA][fG][mA][fU][mU][fC][m |
| | 24207 | | C][fC][Ps][mU][Ps][fC][Ps][mC][Ps][fA][Ps][mC][Ps][mG][fG][mG][fA |
| | | | ][mA][fU][mC][fU][mG][fC][mA][fC][Ps][mU][Ps][fA][3xGalNac] |
| 1008 | 14208_ | C5-m-58 | [5Phos][mU][Ps][fU][Ps][mC][fC][mA][fU][mU][fG][mU][fC][mA][fU][m |
| | 24208 | | A][fG][Ps][mG][Ps][fU][Ps][mU][Ps][fA][Ps][mU][Ps][mC][fU][mA][fU |
| | | | ][mG][fA][mC][fA][mA][fU][mG][fG][Ps][mA][Ps][fA][3xGalNac] |
| 1009 | 14209_ | C5-m-59 | [5Phos][mU][Ps][fG][Ps][mA][fC][mA][fU][mC][fU][mU][fU][mG][fA][m |
| | 24209 | | A][fC][Ps][mA][Ps][fC][Ps][mC][Ps][fU][Ps][mU][Ps][mG][fU][mU][fC |
| | | | ][mA][fA][mA][fG][mA][fU][mG][fU][Ps][mC][Ps][fA][3xGalNac] |
| 1010 | 14210_ | C5-m-60 | [5Phos][mU][Ps][fC][Ps][mA][fU][mU][fG][mU][fC][mA][fU][mA][fG][m |
| | 24210 | | G][fU][Ps][mU][Ps][fA][Ps][mU][Ps][fU][Ps][mG][Ps][mA][fC][mC][fU |
| | | | ][mA][fU][mG][fA][mC][fA][mA][fU][Ps][mG][Ps][fA][3xGalNac] |
| 1011 | 14211_ | C5-m-61 | [5Phos][mU][Ps][fG][Ps][mA][fA][mG][fA][mG][fA][mA][fA][mU][fC][m |
| | 24211 | | C][fA][Ps][mU][Ps][fU][Ps][mG][Ps][fU][Ps][mC][Ps][mU][fG][mG][fA |
| | | | ][mU][fU][mU][fC][mU][fC][mU][fU][Ps][mC][Ps][fA][3xGalNac] |
| 1012 | 14212_ | C5-m-62 | [5Phos][mU][Ps][fA][Ps][mC][fA][mU][fA][mU][fU][mC][fU][mU][fU][m |
| | 24212 | | A][fA][Ps][mC][Ps][fU][Ps][mU][Ps][fC][Ps][mA][Ps][mU][fU][mA][fA |
| | | | ][mA][fG][mA][fA][mU][fA][mU][fG][Ps][mU][Ps][fA][3xGalNac] |
| 1013 | 14213_ | C5-m-63 | [5Phos][mU][Ps][fA][Ps][mG][fU][mC][fC][mU][fU][mU][fU][mA][fC][m |
| | 24213 | | A][fC][Ps][mU][Ps][fC][Ps][mA][Ps][fA][Ps][mA][Ps][mG][fU][mG][fU |
| | | | ][mA][fA][mA][fA][mG][fG][mA][fC][Ps][mU][Ps][fA][3xGalNac] |
| 1014 | 14214_ | C5-m-64 | [5Phos][mU][Ps][fA][Ps][mU][fU][mU][fU][mC][fC][mU][fU][mG][fA][m |
| | 24214 | | A][fA][Ps][mG][Ps][fA][Ps][mU][Ps][fC][Ps][mC][Ps][mU][fU][mU][fC |
| | | | ][mA][fA][mG][fG][mA][fA][mA][fA][Ps][mU][Ps][fA][3xGalNac] |
| 1015 | 14215_ | C5-m-65 | [5Phos][mU][Ps][fG][Ps][mA][fA][mA][fU][mU][fG][mU][fA][mU][fU][m |
| | 24215 | | U][fU][Ps][mA][Ps][fU][Ps][mC][Ps][fU][Ps][mG][Ps][mA][fA][mA][fA |
| | | | ][mU][fA][mC][fA][mA][fU][mU][fU][Ps][mC][Ps][fA][3xGalNac] |
| 1016 | 14216_ | C5-m-66 | [5Phos][mU][Ps][fG][Ps][mU][fA][mA][fU][mU][fU][mC][fA][mA][fA][m |
| | 24216 | | A][fU][Ps][mU][Ps][fC][Ps][mU][Ps][fU][Ps][mA][Ps][mA][fU][mU][fU |
| | | | ][mU][fG][mA][fA][mA][fU][mU][fA][Ps][mC][Ps][fA][3xGalNac] |
| 1017 | 14217_ | C5-m-67 | [5Phos][mU][Ps][fA][Ps][mA][fA][mA][fU][mU][fC][mU][fU][mA][fA][m |
| | 24217 | | A][fG][Ps][mU][Ps][fU][Ps][mC][Ps][fU][Ps][mU][Ps][mC][fU][mU][fU |
| | | | ][mA][fA][mG][fA][mA][fU][mU][fU][Ps][mU][Ps][fA][3xGalNac] |
| 1018 | 14218_ | C5-m-68 | [5Phos][mU][Ps][fG][Ps][mA][fA][mU][fU][mU][fU][mG][fG][mU][fU][m |
| | 24218 | | C][fU][Ps][mG][Ps][fC][Ps][mU][Ps][fC][Ps][mU][Ps][mA][fG][mA][fA |
| | | | ][mC][fC][mA][fA][mA][fA][mU][fU][Ps][mC][Ps][fA][3xGalNac] |
| 1019 | 14219_ | C5-m-69 | [5Phos][mU][Ps][fG][Ps][mU][fC][mA][fU][mU][fU][mU][fA][mU][fA][m |
| | 24219 | | A][fU][Ps][mU][Ps][fA][Ps][mU][Ps][fG][Ps][mU][Ps][mA][fU][mU][fA |
| | | | ][mU][fA][mA][fA][mA][fU][mG][fA][Ps][mC][Ps][fA][3xGalNac] |
| 1020 | 14220_ | C5-m-70 | [5Phos][mU][Ps][fC][Ps][mA][fA][mA][fU][mC][fC][mU][fG][mU][fA][m |
| | 24220 | | C][fU][Ps][mG][Ps][fA][Ps][mC][Ps][fA][Ps][mA][Ps][mA][fG][mU][fA |
| | | | ][mC][fA][mG][fG][mA][fU][mU][fU][Ps][mG][Ps][fA][3xGalNac] |
| 1021 | 14221_ | C5-m-71 | [5Phos][mU][Ps][fG][Ps][mA][fU][mA][fA][mC][fU][mU][fU][mU][fA][m |
| | 24221 | | A][fU][Ps][mA][Ps][fG][Ps][mA][Ps][fG][Ps][mA][Ps][mA][fU][mU][fA |
| | | | ][mA][fA][mA][fG][mU][fU][mA][fU][Ps][mC][Ps][fA][3xGalNac] |
| 1022 | 14222_ | C5-m-72 | [5Phos][mU][Ps][fU][Ps][mU][fA][mA][fG][mU][fC][mU][fU][mC][fU][mC |
| | 24222 | | ][fU][Ps][mU][Ps][fA][Ps][mU][Ps][fU][Ps][mC][Ps][mA][fG][mA][fG] |
| | | | [mA][fA][mG][fA][mC][fU][mU][fA][Ps][mA][Ps][fA][3xGalNac] |
| 1023 | 14223_ | C5-m-73 | [5Phos][mU][Ps][fA][Ps][mU][fA][mA][fU][mU][fC][mC][fA][mA][fU][m |
| | 24223 | | A][fU][Ps][mG][Ps][fA][Ps][mU][Ps][fC][Ps][mA][Ps][mA][fU][mA][fU |
| | | | ][mU][fG][mG][fA][mA][fU][mU][fA][Ps][mU][Ps][fA][3xGalNac] |
| 1024 | 14224_ | C5-m-74 | [5Phos][mU][Ps][fG][Ps][mA][fU][mA][fA][mA][fU][mG][fA][mA][fC][m |
| | 24224 | | A][fU][Ps][mG][Ps][fG][Ps][mC][Ps][fC][Ps][mU][Ps][mA][fU][mG][fU |
| | | | ][mU][fC][mA][fU][mU][fU][mA][fU][Ps][mC][Ps][fA][3xGalNac] |
| 1025 | 14225_ | C5-m-75 | [5Phos][mU][Ps][fA][Ps][mA][fG][mG][fU][mU][fC][mA][fU][mC][fA][m |
| | 24225 | | U][fU][Ps][mU][Ps][fU][Ps][mC][Ps][fU][Ps][mU][Ps][mA][fA][mU][fG |
| | | | ][mA][fU][mG][fA][mA][fC][mC][fU][Ps][mU][Ps][fA][3xGalNac] |
| 1026 | 14226_ | C5-m-76 | [5Phos][mU][Ps][fG][Ps][mG][fA][mA][fG][mU][fG][mC][fU][mA][fU][m |
| | 24226 | | A][fA][Ps][mA][Ps][fA][Ps][mC][Ps][fA][Ps][mU][Ps][mU][fU][mA][fU |
| | | | ][mA][fG][mC][fA][mC][fU][mU][fC][Ps][mC][Ps][fA][3xGalNac] |
| 1027 | 14227_ | C5-m-77 | [5Phos][mU][Ps][fC][Ps][mA][fA][mG][fU][mA][fC][mU][fC][mU][fU][m |
| | 24227 | | A][fA][Ps][mA][Ps][fG][Ps][mC][Ps][fA][Ps][mA][Ps][mU][fU][mA][fA |
| | | | ][mG][fA][mG][fU][mA][fC][mU][fU][Ps][mG][Ps][fA][3xGalNac] |
| 1028 | 14228_ | C5-m-78 | [5Phos][mU][Ps][fC][Ps][mC][fA][mA][fU][mG][fA][mU][fU][mU][fC][m |
| | 24228 | | C][fU][Ps][mG][Ps][fU][Ps][mU][Ps][fU][Ps][mC][Ps][mA][fG][mG][fA |
| | | | ][mA][fA][mU][fC][mA][fU][mU][fG][Ps][mG][Ps][fA][3xGalNac] |
| 1029 | 14229_ | C5-m-79 | [5Phos][mU][Ps][fA][Ps][mU][fG][mG][fU][mA][fU][mA][fU][mU][fC][m |
| | 24229 | | A][fU][Ps][mU][Ps][fU][Ps][mC][Ps][fC][Ps][mA][Ps][mA][fU][mG][fA |
| | | | ][mA][fU][mA][fU][mA][fC][mC][fA][Ps][mU][Ps][fA][3xGalNac] |
| 1030 | 14230_ | C5-m-80 | [5Phos][mU][Ps][fA][Ps][mA][fC][mA][fA][mG][fA][mU][fG][mA][fA][m |
| | 24230 | | C][fU][Ps][mU][Ps][fC][Ps][mC][Ps][fC][Ps][mA][Ps][mA][fG][mU][fU] |
| | | | [mC][fA][mU][fC][mU][fU][mG][fU][Ps][mU][Ps][fA][3xGalNac] |
| 1031 | 14231_ | C5-m-81 | [5Phos][mU][Ps][fG][Ps][mA][fA][mC][fU][mU][fC][mA][fG][mG][fA][m |
| | 24231 | | A][fU][Ps][mU][Ps][fU][Ps][mU][Ps][fA][Ps][mG][Ps][mA][fU][mU][fC] |
| | | | [mC][fU][mG][fA][mA][fG][mU][fU][Ps][mC][Ps][fA][3xGalNac] |
| 1032 | 14232_ | C5-m-82 | [5Phos][mU][Ps][fA][Ps][mG][fU][mC][fU][mU][fC][mU][fC][mU][fU][m |
| | 24232 | | A][fU][Ps][mU][Ps][fC][Ps][mC][Ps][fA][Ps][mA][Ps][mA][fU][mA][fA |
| | | | ][mG][fA][mG][fA][mA][fG][mA][fC][Ps][mU][Ps][fA][3xGalNac] |
| 1033 | 14233_ | C5-m-83 | [5Phos][mU][Ps][fA][Ps][mA][fU][mG][fU][mU][fU][mA][fU][mA][fC][mU |
| | 24233 | | ][fU][Ps][mU][Ps][fG][Ps][mA][Ps][fU][Ps][mA][Ps][mA][fA][mG][fU] |
| | | | [mA][fU][mA][fA][mA][fC][mA][fU][Ps][mU][Ps][fA][3xGalNac] |
| 1034 | 14234_ | C5-m-84 | [5Phos][mU][Ps][fC][Ps][mG][fG][mA][fA][mU][fC][mG][fU][mA][fC][m |
| | 24234 | | A][fC][Ps][mA][Ps][fA][Ps][mA][Ps][fG][Ps][mG][Ps][mG][fU][mG][fU |
| | | | ][mA][fC][mG][fA][mU][fU][mC][fC][Ps][mG][Ps][fA][3xGalNac] |
| 1035 | 14235_ | C5-m-85 | [5Phos][mU][Ps][fA][Ps][mU][fA][mC][fC][mU][fC][mU][fG][mC][fU][m |
| | 24235 | | C][fU][Ps][mU][Ps][fC][Ps][mU][Ps][fG][Ps][mA][Ps][mA][fG][mA][fG |
| | | | ][mC][fA][mG][fA][mG][fG][mU][fA][Ps][mU][Ps][fA][3xGalNac] |
| 1036 | 14236_ | C5-m-86 | [5Phos][mU][Ps][fA][Ps][mU][fC][mA][fA][mU][fU][mU][fC][mU][fU][m |
| | 24236 | | C][fU][Ps][mA][Ps][fC][Ps][mC][Ps][fA][Ps][mU][Ps][mA][fG][mA][fA |
| | | | ][mG][fA][mA][fA][mU][fU][mG][fA][Ps][mU][Ps][fA][3xGalNac] |
| 1037 | 14237_ | C5-m-87 | [5Phos][mU][Ps][fA][Ps][mA][fC][mA][fU][mU][fG][mU][fG][mU][fU][m |
| | 24237 | | U][fU][Ps][mG][Ps][fC][Ps][mA][Ps][fU][Ps][mU][Ps][mA][fA][mA][fA |
| | | | ][mC][fA][mC][fA][mA][fU][mG][fU][Ps][mU][Ps][fA][3xGalNac] |
| 1038 | 14238_ | C5-m-88 | [5Phos][mU][Ps][fA][Ps][mA][fC][mU][fU][mU][fA][mU][fA][mA][fG][m |
| | 24238 | | C][fA][Ps][mU][Ps][fA][Ps][mU][Ps][fG][Ps][mC][Ps][mU][fG][mC][fU |
| | | | ][mU][fA][mU][fA][mA][fA][mG][fU][Ps][mU][Ps][fA][3xGalNac] |
| 1039 | 14239_ | C5-m-89 | [5Phos][mU][Ps][fA][Ps][mG][fG][mA][fU][mA][fA][mC][fU][mU][fU][m |
| | 24239 | | U][fA][Ps][mA][Ps][fU][Ps][mA][Ps][fG][Ps][mA][Ps][mU][fA][mA][fA |
| | | | ][mA][fG][mU][fU][mA][fU][mC][fC][Ps][mU][Ps][fA][3xGalNac] |
| 1040 | 14240_ | C5-m-90 | [5Phos][mU][Ps][fU][Ps][mU][fU][mA][fU][mU][fG][mG][fU][mU][fG][m |
| | 24240 | | A][fU][Ps][mA][Ps][fC][Ps][mU][Ps][fG][Ps][mU][Ps][mA][fU][mC][fA |
| | | | ][mA][fC][mC][fA][mA][fU][mA][fA][Ps][mA][Ps][fA][3xGalNac] |
| 1041 | 14241_ | C5-m-91 | [5Phos][mU][Ps][fG][Ps][mC][fA][mA][fC][mU][fG][mU][fU][mU][fU][m |
| | 24241 | | C][fU][Ps][mU][Ps][fC][Ps][mU][Ps][fG][Ps][mG][Ps][mA][fG][mA][fA |
| | | | ][mA][fA][mC][fA][mG][fU][mU][fG][Ps][mC][Ps][fA][3xGalNac] |
| 1042 | 14242_ | C5-m-92 | [5Phos][mU][Ps][fG][Ps][mC][fU][mU][fU][mG][fA][mU][fA][mC][fA][m |
| | 24242 | | A][fC][Ps][mU][Ps][fU][Ps][mC][Ps][fC][Ps][mA][Ps][mG][fU][mU][fG |
| | | | ][mU][fA][mU][fC][mA][fA][mA][fG][Ps][mC][Ps][fA][3xGalNac] |
| 1043 | 14243_ | C5-m-93 | [5Phos][mU][Ps][fC][Ps][mA][fA][mA][fG][mC][fU][mU][fC][mU][fC][m |
| | 24243 | | U][fC][Ps][mU][Ps][fU][Ps][mC][Ps][fA][Ps][mA][Ps][mG][fA][mG][fA] |
| | | | [mG][fA][mA][fG][mC][fU][mU][fU][Ps][mG][Ps][fA][3xGalNac] |
| 1044 | 14244_ | C5-m-94 | [5Phos][mU][Ps][fG][Ps][mG][fA][mA][fC][mU][fC][mC][fU][mU][fU][m |
| | 24244 | | C][fG][Ps][mU][Ps][fC][Ps][mU][Ps][fG][Ps][mC][Ps][mC][fG][mA][fA |
| | | | ][mA][fG][mG][fA][mG][fU][mU][fC][Ps][mC][Ps][fA][3xGalNac] |
| 1045 | 14245_ | C5-m-95 | [5Phos][mU][Ps][fA][Ps][mU][fG][mA][fC][mA][fG][mU][fU][mC][fU][m |
| | 24245 | | U][fU][Ps][mG][Ps][fA][Ps][mC][Ps][fU][Ps][mG][Ps][mA][fA][mA][fG |
| | | | ][mA][fA][mC][fU][mG][fU][mC][fA][Ps][mU][Ps][fA][3xGalNac] |
| 1046 | 14246_ | C5-m-96 | [5Phos][mU][Ps][fU][Ps][mG][fC][mA][fG][mA][fA][mU][fA][mA][fC][m |
| | 24246 | | A][fU][Ps][mG][Ps][fU][Ps][mC][Ps][fC][Ps][mA][Ps][mA][fU][mG][fU |
| | | | ][mU][fA][mU][fU][mC][fU][mG][fC][Ps][mA][Ps][fA][3xGalNac] |
| 1047 | 14247_ | C5-m-97 | [5Phos][mU][Ps][fA][Ps][mG][fA][mA][fG][mU][fC][mC][fU][mA][fU][m |
| | 24247 | | A][fG][Ps][mU][Ps][fU][Ps][mG][Ps][fU][Ps][mA][Ps][mC][fU][mA][fU |
| | | | ][mA][fG][mG][fA][mC][fU][mU][fC][Ps][mU][Ps][fA][3xGalNac] |
| 1048 | 14248_ | C5-m-98 | [5Phos][mU][Ps][fA][Ps][mU][fA][mA][fC][mU][fU][mU][fU][mA][fA][m |
| | 24248 | | U][fA][Ps][mG][Ps][fA][Ps][mG][Ps][fA][Ps][mU][Ps][mU][fA][mU][fU |
| | | | ][mA][fA][mA][fA][mG][fU][mU][fA][Ps][mU][Ps][fA][3xGalNac] |
| 1049 | 14249_ | C5-m-99 | [5Phos][mU][Ps][fC][Ps][mU][fA][mA][fG][mA][fU][mU][fU][mC][fU][m |
| | 24249 | | U][fU][Ps][mU][Ps][fC][Ps][mC][Ps][fA][Ps][mA][Ps][mA][fA][mA][fG |
| | | | ][mA][fA][mA][fU][mC][fU][mU][fA][Ps][mG][Ps][fA][3xGalNac] |
| 1050 | 14250_ | C5-m-100 | [5Phos][mU][Ps][fA][Ps][mU][fA][mA][fA][mU][fG][mA][fA][mC][fA][m |
| | 24250 | | U][fG][Ps][mG][Ps][fC][Ps][mC][Ps][fU][Ps][mG][Ps][mC][fA][mU][fG |
| | | | ][mU][fU][mC][fA][mU][fU][mU][fA][Ps][mU][Ps][fA][3xGalNac] |

Note =
each of the above constructs may or may not have a phosphate modification at the 5′ end group. Furthermore, and independently, each of the above constructs may or may not have a “3x GalNAc” coupled to the 3′ end group. Optional are constructs with a 3x GalNAc ligand, in particular a toothbrush ligand as defined herein. Particularly optional are constructs which in addition have a 5′ phosphate, even though this is not a strict requirement, given that in the absence thereof, mammalian cells will add such phosphate in case it is absent from the molecule as administered.

Specific notes about the nomenclature in Tables 3a to 3c:
fN: 2′-Fluoro residues
mN: 2′-O-methyl residues
Ps: phosphorothioate
p, Phos: phosphate
(GalNAc): Sirnaomics mono-GalNAc building block

It should also be noted that the scope of the present disclosure extends to sequences that correspond to those in the Tables above, and wherein the 5′ terminal nucleoside of the antisense (guide) strand (first region as defined in the claims herein) can include any nucleobase that can be present in an RNA molecule, in other words can be any of adenine (A), uracil (U), guanine (G) or cytosine (C). Additionally, the scope of the present disclosure extends to sequences that correspond to those in the Tables above, and wherein the 3′ terminal nucleoside of the sense (passenger) strand (second region as defined in the claims herein) can include any nucleobase that can be present in an RNA molecule, in other words can be any of adenine (A), uracil (U), guanine (G) or cytosine (C), optionally however a nucleobase that is complementary to the 5′ nucleobase of the antisense (guide) strand (first region as defined in the claims herein).

While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the Examples described above may be combined with aspects of any of the other Examples described to form further Examples.

It will be understood that the above description of an optional embodiment is given by way of example only and that various modifications may be made by those skilled in the art. What has been described above includes Examples of one or more embodiments. It is, of course, not possible to describe every conceivable modification and alteration of the above compounds, compositions or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

### EXAMPLES

The following Examples illustrate certain embodiments of the present disclosure and are not limiting. Moreover, where specific embodiments are provided, the inventors have contemplated generic application of those specific embodiments. For example, disclosure of an oligonucleotide having a particular motif or modification patterns provides reasonable support for additional oligonucleotides having the same or similar motif or modification patterns.

The syntheses of the RNAi constructs according to the present disclosure and disclosed herein have been carried out using synthesis methods known to the person skilled in the art, such as synthesis methods disclosed in https://en.wikipedia.org/wiki/Oligonucleotide_synthesis {retrieved on 16 Feb. 2022}, wherein the methods disclosed on this website are incorporated by reference herein in their entirety. The only difference to the synthesis method disclosed in this reference is that GalNAc phosphoramidite immobilized on a support is used in the synthesis method during the first synthesis step.

### Example 1

### Materials and Methods

### Cell Culture:

Human primary hepatocytes (5 donor pooled-Sekisui XenoTech, HPCH05+) were thawed immediately prior to experimentation and cultured in 1× complete Williams medium (Gibco, A1217601) supplemented with Hepatocytes plating supplement pack (Gibco, CM3000). FBS concentration was modified from manufacture recipe to a final 2.5% (as opposed to 5%) for compound stability.

1× Complete WEM: 2.5% FBS, 1 μM Dexamethasone, Pen/Strep (100 U/mL/100 μg/mL), 4 μg/ml Human Insulin, 2 mM GlutaMAX, 15 mM HEPES, pH 7.4.

### C5 Target Identification and Duplex Preparation:

Oligomeric compounds targeting C5 were identified by bioinformatic analysis on human C5 mRNA sequence as given in RefSeq sequence ID NM_001735.2. 100 compounds were selected for synthesis as mxRNA hairpins. Compounds were dissolved to 50 μM in molecular biology grade water. Duplexes were annealed by heating at 95° C. for 5 minutes followed by gradual cooling to room temperature. mxRNAs were annealed by heating at 95° C. for 5 minutes followed by rapid cooling on ice.

### C5—Primary Screen:

On the day of transfection, primary human hepatocytes were thawed in 45 mL of human OptiThaw (Sekisui XenoTech, K8000) and centrifuged down at 200 g for 5 minutes. Cells were resuspended in 2× complete WEM and counted. Cells were then plated in 50 μL of 2× complete WEM at 25,000 cells per well on 96 well type 1 rat tail Collagen plates and allowed to rest and attach for four hours before transfection. After rest, the compounds were diluted further to 2 μM in basal WEM. 50 μL of each 2 μM compound was added to respective triplicates of the plated hepatocytes for a final concentration of 1 μM in a volume of 100 μL 1× complete WEM.

72 hours post transfection, cells were harvested, and RNA isolated using the PureLink Pro 96 total RNA Purification Kit (ThermoFisher, 12173011A) according to the manufacturer protocol. Harvested RNA was assayed for C5 expression via Taqman qPCR using the Luna Universal Probe One-Step RT-qPCR Kit (NEB, E3006). A qPCR assay was performed for each sample using a C5 TaqMan probe set (Hs01004342_m1-FAM) multiplexed with a common GAPDH VIC probe (ThermoFisher, 4326317E). Thermocycling and data acquisition was performed with an Applied Biosystems QuantStudio 3/5 Real-Time PCR System.

### C5—Secondary Screen:

Based on data from the primary screen, a narrower set of the best performing 25 C5-targeting mxRNA constructs were tested in dose curves. Compounds were diluted further to 2 μM in basal WEM. A seven step, five-fold dilution series was prepared in basal WEM from 2 μM to 0.000128 μM. 50 μL of each dilution was added to respective triplicates of the plated hepatocytes for a final dilution series of 1 μM down to 0.000064 M in a volume of 100 μL 1× complete WEM.

72 hours post transfection, cells were harvested, and RNA isolated using the PureLink Pro 96 total RNA Purification Kit (ThermoFisher, 12173011A) according to the manufacturer protocol. Harvested RNA was assayed for C5 expression via Taqman qPCR using the Luna Universal Probe One-Step RT-qPCR Kit (NEB, E3006). A qPCR assay was performed for each sample using a C5 TaqMan probe set (Hs01004342_m1-FAM) multiplexed with a common GAPDH VIC probe (ThermoFisher, 4326317E). Thermocycling and data acquisition was performed with an Applied Biosystems QuantStudio 3/5 Real-Time PCR System.

### Example 2

### Results

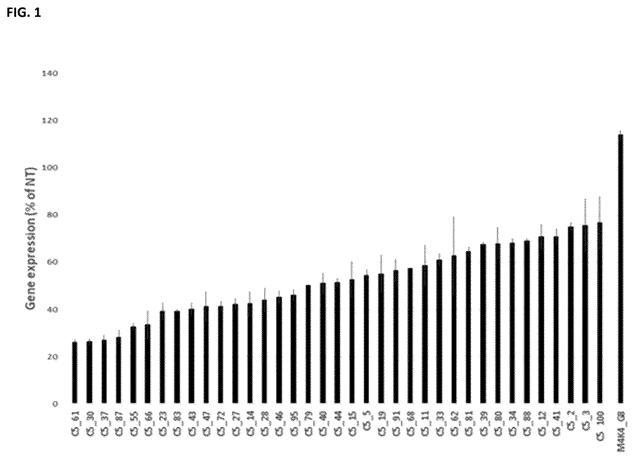

FIG. 1 shows results of the primary screening of selected compounds according to the present disclosure and their activity in inhibiting C5 expression.

Table 4 below shows IC50 values (in nM) for 25 optional constructs selected in accordance with the Examples. Max % KD indicates the maximally achieved knock-down at 1000 nM with 0% being no knock-down and 100% full knock-down. M4K4 was used as reference.

| Experimental | | |
|---|---|---|
| denotiation | Max % KD | IC50 |
| C5-m-30 | 71.88574381 | 4.943 |
| C5-m-37 | 77.48131233 | 11.25 |
| C5-m-83 | 59.57363723 | 21.99 |
| C5-m-61 | 68.93837532 | 40.04 |
| C5-m-74 | 63.30023809 | 41.25 |
| C5-m-82 | 62.26208156 | 68.84 |
| C5-m-87 | 63.87542555 | 89.55 |
| C5-m-55 | 63.0068919 | 90.72 |
| C5-m-23 | 60.00147173 | 213.2 |
| C5-m-28 | 50.59311869 | 295.4 |
| C5-m-42 | 49.72928101 | 302.6 |
| C5-m-73 | 48.77874599 | 351.7 |
| C5-m-66 | 56.30345942 | 351.9 |
| C5-m-47 | 53.46576404 | 367.2 |
| C5-m-46 | 50.25328616 | 416.7 |
| C5-m-27 | 48.71998765 | 583.3 |
| C5-m-16 | 50.77488697 | 629.7 |
| C5-m-43 | 43.01211645 | 779.6 |
| C5-m-36 | 48.1079184 | 894.2 |
| C5-m-72 | 44.02312363 | 1090 |
| C5-m-53 | 40.3528029 | 1167 |
| C5-m-14 | 38.57830035 | 2282 |
| C5-m-75 | 38.99786713 | 3485 |
| C5-m-59 | 32.76891127 | 5167 |
| C5-m-56 | 22.82809496 | 6117 |
| M4K4 | 11.680749 | 3810 |

The IC50 data in the single- to low double-digit nanomolar range demonstrate outstanding performance of numerous constructs of the disclosure. Furthermore, no obvious toxicity was observed.

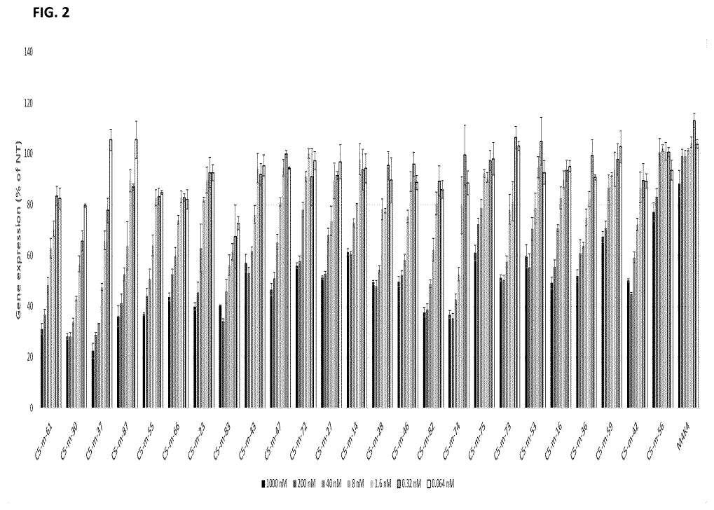

Further results of the screening and the outstanding performance of the above inventive constructs in Table 4 above are shown in FIG. 2.

Table 5 below shows IC50 values (in nM) for 6 optional constructs selected in accordance with the Examples. Max % KD indicates the maximally achieved knock-down at 1000 nM with 0% being no knock-down and 100% full knock-down.

| | Experimental | | |
|---|---|---|---|
| SEQ ID No. | denotation | KD % at 1000 nM | IC50 (nM) |
| 980 | C5-30 (large) | 85.136214 | 2.939 |
| 987 | C5-37 (large) | 85.717658 | 5.605 |
| 1033 | C5-83 (large) | 71.592597 | 37.45 |

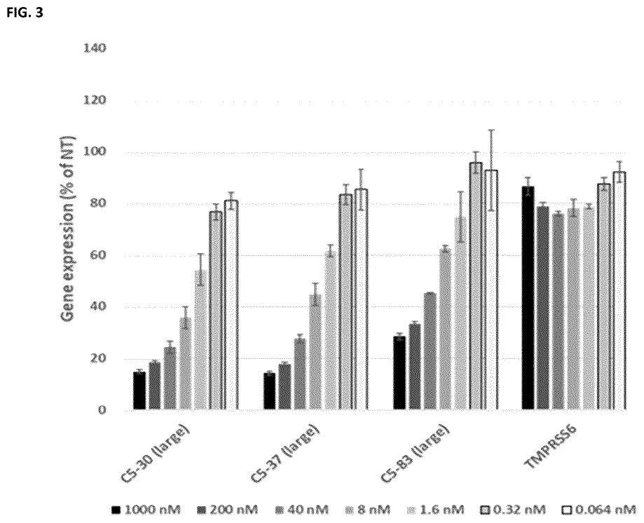

Further results of the constructs in Table 5 above with different concentrations are shown in FIG. 3.

Table 5 and FIG. 3 show C5 large scale preparations mirrored the screening synthesis very closely. C5-m-30 and C5-m-37 had 85% knockdowns and C5-m-83 had a 72% knockdown at the highest dose.

### Example 3

Complement component C5 targeting mxRNA Leads for Candidate Dose and Duration Response study in humanized liver-uPA-SCID mice) model, non-GLP

### 1. Study Objective(s)

The objective of this non-GLP study is to evaluate the dose and duration response of GalNAc conjugated complement component C5 targeting mxRNA constructs in humanized liver-uPA-SCID. The compound(s) will be administered subcutaneously, and the mice will be survived for up to 42 days.

Prior to necropsy, plasma and serum will be collected. At necropsy, 3 liver biopsies (2 mm) per animal will be preserved in separate vials in RNAlater, flash frozen, and stored at −80° C. Three more liver biopsies (2 mm) will be taken, flash frozen in the same vial, and stored at −80° C.

### 2. Regulatory Compliance

This non-GLP study will not be conducted in accordance with the Food and Drug Administration's Good Laboratory Practice (GLP) regulations (21 CFR Part 58).

### 3. Animal Welfare Compliance

This protocol has been reviewed and approved by the Test Facility IACUC Committec.

### 4. Test System Information

### 4.1. Animal Test

4.1.1. Common Name: Mouse
4.1.2. Breed/Class: Rodent-humanized liver-uPA-SCID mice model
4.1.3. Number of Animals (by gender): 44 Male all naïve

### 4.2. Acclimation Period:

4.2.1. Duration:

All animals will be acclimated for a minimum period of five (5) days prior to release by the Attending veterinarian, at which time the overall health of the animals will be evaluated.
4.2.2. Required medication and/or vaccination:
   All rodents received will come from a vendor that is certified to be free of any lethal parasites that may affect the facility's total colony.
   All rodents must be accompanied by a sentinel report including statistical analysis.
   Each shipment of rodents must be housed separately from others in the facility.

### 4.3. Animal Identification Method and Location:

Animals will be assigned sequential numbers. The animals will be car notched by the vendor prior to shipment to permanently identify each animal. Animals may have color markings to distinguish between similar ear notches. A cage card will also be affixed to each animal cage denoting the animal number, gender, vendor, strain, study director, and study number.

### 5. Study Design

### 5.1. Design Details

This study will have one type of mice, N=44. Animals will be grouped by treatment type, dosage, and survival period. Each animal will be treated by subcutaneous injection of test material. See study table 1 for details.

At necropsy, three 2 mm biopsy punches will be taken from the left, middle and right liver lobes, placed in separate vials, soaked in RNAlater for 15 minutes, flash frozen and stored at −80° C. Another three 2 mm liver biopsies from the left, middle and right liver lobes will be placed into one vial, flash frozen and stored at −80° C. The rest of the liver will be flash frozen and stored in 10 ml conical tubes at −80° C.

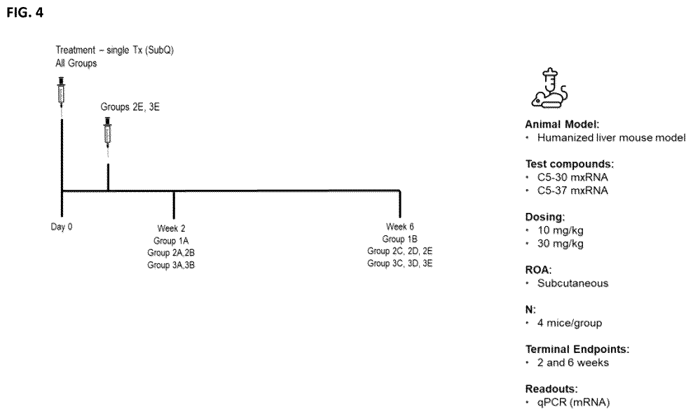

The study schedule is shown in FIG. 4.

TABLE 6
Dose information

| Terminal | Control | C5-30 | C5-37 |
|---|---|---|---|
| Timepoint | (PBS) | (SEQ ID No. 980) | (SEQ ID No. 987) |
| Week 2 | Group | Group 2A | Group 3A |
| | 1A | (10 mg/kg) | (10 mg/kg) |
| | (N = 4) | (N = 4) | (N = 4) |
| | | Group 2B | Group 3B |
| | | (30 mg/kg) | (30 mg/kg) |
| | | (N = 4) | (N = 4) |
| Week 6 | Group | Group 2C | Group 3C |
| | 1B | (10 mg/kg) | (10 mg/kg) |
| | (N = 4) | (N = 4) | (N = 4) |
| | | Group 2D | Group 3D |
| | | (30 mg/kg) | (30 mg/kg) |
| | | (N = 3) | (N = 3) |
| | | Group 2E | Group 3E |
| | | 2x (10 mg/kg) | 2x (10 mg/kg) |
| | | (N = 3) | (N = 3) |

TABLE 7
Study table

| | Number | Treatment | | | Pre- |
|---|---|---|---|---|---|
| | of | Subcutaneous | TX | Survival | Euthanasia |
| Group | Animals | Injection | Daysq | Days | and Necropsy |
| 1A | 4 | Control (PBS) | 0 | 14 | Pre- |
| 1B | 4 | Control (PBS) | 0 | 42 | Euthanasia: |
| 2A | 4 | C5-30 (10 mg/kg) | 0 | 14 | Plasma and |
| 2B | 4 | C5-30 (30 m/kg) | 0 | 14 | serum |
| 2C | 4 | C5-30 (10 m/kg) | 0 | 42 | collection. |
| 2D | 3 | C5-30 (30 m/kg) | 0 | 42 | Necropsy: |
| 2E | 3 | C5-30 (10 m/kg) | 0, 7 | 42 | 2 mm biopsy |
| 3A | 4 | C5-37 (10 mg/kg) | 0 | 14 | of left, middle |
| 3B | 4 | C5-37 (30 mg/kg) | 0 | 14 | and right liver |
| 3C | 4 | C5-37 (10 mg/kg) | 0 | 42 | lobes in |
| 3D | 3 | C5-37 (30 mg/kg) | 0 | 42 | separate |
| 3E | 3 | C5-37 (10 mg/kg) | 0, 7 | 42 | vials, in |
| Spares | 0 | | | | RNAlater for |
| Total | 44 | | | | 15 min, flash |
| | | | | | freeze then |
| | | | | | stored |
| | | | | | at −80° C. |
| | | | | | 2 mm biopsy |
| | | | | | of left, middle |
| | | | | | and right liver |
| | | | | | all in one vial, |
| | | | | | flash freeze |
| | | | | | then stored |
| | | | | | at −80° C. |
| | | | | | Rest of liver, |
| | | | | | flash freeze |
| | | | | | then stored |
| | | | | | at −80° C. |

### 6. Test Article and Ancillary Material Information

### 6.1. Test Drug 1:

6.1.1. Identification: C5-30 (SEQ ID No. 980)
6.1.2. Manufacturer: Sirnaomics
6.1.3. Description: GalNAc conjugated human Complement component C5 targeting mxRNA
6.1.4. Lot/Batch Number: Will be recorded on study materials form.
6.1.5. Expiration Date: Will be recorded on study materials form.
6.1.6. Storage Temperature: 4° C.
6.1.7. Bio-Hazard Status: None
6.1.8. MSDS*: TBD
6.1.9. Appearance: Clear Liquid
6.1.10. Dose Information: See Table 6
6.1.11. Residual Test Article Storage: None

### 6.2. Test Drug 2:

6.2.1. Identification: C5-37
6.2.2. Manufacturer: Sirnaomics
6.2.3. Description: GalNAc conjugated human Complement component C5 targeting mxRNA
6.2.4. Lot/Batch Number: Will be recorded on study materials form.
6.2.5. Expiration Date: Will be recorded on study materials form.
6.2.6. Storage Temperature: 4° C.
6.2.7. Bio-Hazard Status: None
6.2.8. MSDS*: TBD
6.2.9. Appearance: Clear Liquid
6.2.10. Dose Information: See Table 6
6.2.11. Residual Test Article Storage: None

### Results

Results of the C5 gene knock down by constructs C5-30 and C5-37 in humanized liver-uPA-SCID mice are shown in the Tables below.

Table 8a: Results of C5 gene knockdown for construct C5-30 (see Table 3c for structure) at several time points using different doses

| C5-30 (SEQ ID No. 980) | |
|---|---|
| Single Tx - 10 mg/kg | 51% KD at 2 weeks |
| | 10% KD at 6 weeks |
| Single Tx - 30 mg/kg | 61% KD at 2 weeks |
| | 36% KD at 6 weeks |
| Repeat 2x Tx - 10 mg/kg | 17% KD at 6 weeks |

Table 8b: Results of C5 gene knockdown for construct C5-37 (see Table 3c for structure) at several time points using different doses

| C5-37 (SEQ ID No. 987) | |
|---|---|
| Single Tx - 10 mg/kg | 50% KD at 2 weeks |
| | 27% KD at 6 weeks |
| Single Tx - 30 mg/kg | 54% KD at 2 weeks |
| | 47% KD at 6 weeks |
| Repeat 2x Tx - 10 mg/kg | 40% KD at 6 weeks |

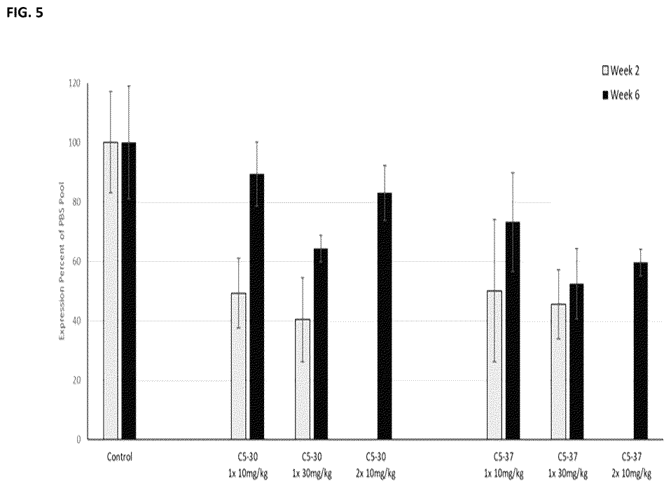

The results of the mouse study are also shown in FIG. 5.

### Example 4

Evaluation of Duration Effect of Human Complement C5 Targeting mxRNA, in the Humanized Liver-uPA-SCID Mouse Model, Non-GLP.

The protocol is represented in its original wording. Therefore, any use of future tense of verbs means that the experiments have already been carried out.

### 1. Study Objective(s)

The objective of this non-GLP study is to evaluate, in humanized liver-uPA-SCID mice the duration effect of C5-30 mxRNA compound (see Table 3c; experimental denotation: C5-m-30 SEQ ID No. 980) targeting human complement C5 mRNA.

The compound(s) will be administered subcutaneously, and the mice will be survived for up to 84 days.

### 2. Study Design

### 2.1. Design Details

This study will have one type of mice, PXB. Animals will be grouped by treatment type, dosage, and survival period. Each animal will be treated by subcutaneous injection of test material. (Note: that the injection must be given subcutaneously. The test articles will not be functional if the subcutaneous site is missed, and injection is given within the muscular region or test articles are injected into the vein/bloodstream).
Group 1A, 1B, 1C, and 1D will have five animals and receive a single control dose of PBS.
Group 2A, 2B, 2C, and 2D will have five animals and receive a single dose of C5-30 (mxRNA targeting human complement C5 mRNA, SEQ ID No. 980) at 30 mg/kg.

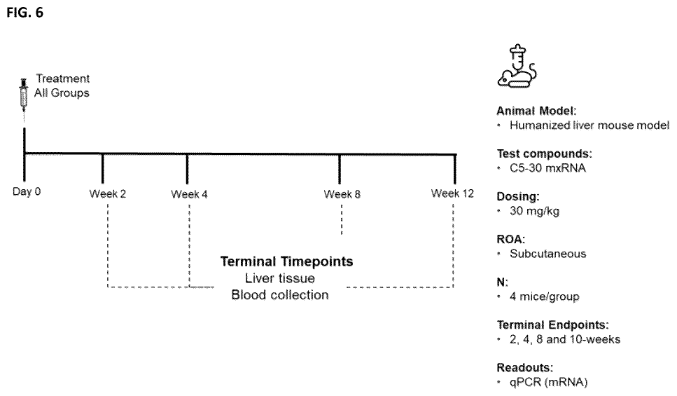

Animals will be survived for 14, 28, 56, and 84 days. See FIG. 6 and Table 9 for details.

TABLE 9
Study Table

| | | | Treatment | | | |
|---|---|---|---|---|---|---|
| | Number | | Subcutaneous | | | |
| | of | Mouse | Injection | Survival | | Pre-Euthanasia and |
| Group | Animals | Type | Day 0 | Days | Blood | Necropsy |
| 1A | 5 | PXB | Control (PBS) | 14 | Blood collected | Pre-Euthanasia: Plasma |
| 1B | 5 | PXB | Control (PBS) | 28 | for plasma. | and collection. |
| 1C | 5 | PXB | Control (PBS) | 56 | Plasma will be | Necropsy: |
| 1D | 5 | PXB | Control (PBS) | 84 | evenly separated | 2 mm biopsy of left, middle |
| 2A | 5 | PXB | C5-30 30 mg/kg | 14 | into two labeled | and right liver lobes in |
| 2B | 5 | PXB | C5-30 30 mg/kg | 28 | vials. | separate vials, in RNAlater |
| 2C | 5 | PXB | C5-30 30 mg/kg | 56 | | for 15 min, flash freeze then |
| 2D | 5 | PXB | C5-30 30 mg/kg | 84 | | stored at −80° C. |
| | | | | | | 2 mm biopsy of left, middle |
| | | | | | | and right liver all in one vial, |
| | | | | | | flash freeze then stored at −80° C. |
| | | | | | | Rest of liver, flash freeze |
| | | | | | | then stored at −80° C. |

Note = The structure of C5-30 is shown in Table 3c under the experimental denotation “C5-m-30”.

### 3. Test Article and Ancillary Material Information

### 3.1. Test Drug 1:

3.1.1. Identification: C5-30
3.1.2. Manufacturer: Sirnaomics
3.1.3. Description: GalNAc-mxRNA targeting human complement C5 mRNA
3.1.4. Lot/Batch Number: Will be recorded on study materials form.
3.1.5. Expiration Date: Will be recorded on study materials form.
3.1.6. Storage Temperature: 4° C.
3.1.7. Bio-Hazard Status: None
3.1.8. SDS*: TBD
3.1.9. Appearance: Clear Liquid
3.1.10. Dose Information: See Table 1
3.1.11. Residual Test Article Storage: None
   Note=C5-30 is shown in Table 3c under the experimental denotation C5-m-30.

### Results

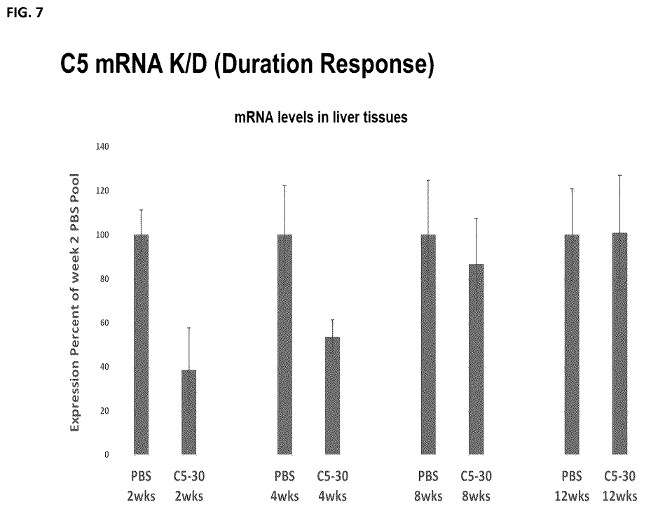

The results of the study are shown in FIG. 7.

FIG. 7 shows the following duration response in terms of knock down (KD) of the C5 gene expression:
61% KD of C5 mRNA at week 2;
46% KD of C5 mRNA at week 4;
   13% KD of C5 mRNA at week 8; and
   Return to control levels at week 12

### Note:

2 animals from control group (8 wk) were found dead (remaining N=4)
2 animals from C5-30 (4 wk) group were found dead (remaining N=4)
2 animals from C5-30 (8 wk) group were found dead (remaining N=4)

1. An oligomeric compound for inhibiting expression of complement component C5, wherein the compound comprises a single nucleotide strand, wherein said single strand consists of a modified or unmodified oligonucleotide comprising a first nucleobase sequence selected from the group consisting of SEQ ID NOs: 1 to 250 linked directly to a second nucleobase sequence selected respectively from the group consisting of SEQ ID NOs: 251 to 500, wherein said single strand has a total length of 33 or 34 nucleosides.

2. (canceled)

3. The oligomeric compound according to claim 1, wherein the modified or unmodified nucleotide has a nucleobase sequence that consists of: SEQ ID NOs: 61 and 311, 30 and 280, 37 and 287, 87 and 337, 55 and 305, 66 and 316, 23 and 273, 83 and 333, 43 and 293, 47 and 297, 72 and 322, 27 and 277, 14 and 264, 28 and 278, 46 and 296, 82 and 332, 74 and 324, 75 and 325, 73 and 323, 53 and 303, 16 and 266, 36 and 286, 59 and 309, 42 and 292, and 56 and 306, or a portion thereof.

4-12. (canceled)

13. The oligomeric compound according to claim 1, which further comprises one or more ligands.

14. The oligomeric compound according to claim 13, wherein said one or more ligands are conjugated to the second region of linked nucleosides and/or the first region of linked nucleosides.

15. The oligomeric compound according to claim 14, wherein said one or more ligands are conjugated at the 3′ terminal nucleoside of the second region of linked nucleosides and/or the 5′ terminal nucleoside of the second region of linked nucleosides.

16. The oligomeric compound according to claim 13, wherein said one or more ligands bind cellular membrane or a specific target on cellular surface.

17-20. (canceled)

21. The oligomeric compound according to claim 16, wherein said one or more ligands comprise one or more N-Acetyl-Galactosamine moieties.

21-23. (canceled)

24. The oligomeric compound according to claim 21, wherein the one or more N-Acetyl-Galactosamine moieties are attached to the oligomeric compound as a biantennary or triantennary configuration.

25-31. (canceled)

32. The oligomeric compound according to claim 1, wherein said single strand has a nucleobase sequence selected from the group consisting of SEQ ID NOs: 561, 530, 537, 587, 555, 566, 523, 583, 543, 547, 572, 527, 514, 528, 546, 582, 574, 575, 573, 553, 516, 536, 559, 542, and 556.

33. The oligomeric compound according to claim 32, wherein the single strand is selected from the group consisting of SEQ ID NOs: 1011, 980, 987, 1037, 1005, 1016, 973, 1033, 993, 997, 1022, 977, 964, 978, 996, 1032, 1024, 1025, 1023, 1003, 966, 986, 1009, 992, 1006.

34-36. (canceled)

37. The oligomeric compound according to claim 1, which comprises internucleoside linkages and wherein at least one internucleoside linkage is a modified internucleoside linkage.

38-39. (canceled)

40. The oligomeric compound according to claim 37, which comprises 7, 8, 9 or 10 phosphorothioate or phosphorodithioate internucleoside linkages.

41-44. (canceled)

45. The oligomeric compound according to claim 1, wherein at least one nucleoside comprises a modified sugar selected from the group consisting of a 2′-O-alkyl modified sugar, 2′-O-methyl modified sugar, 2′-O-methoxyethyl modified sugar, 2′-O-allyl modified sugar, 2′-C-allyl modified sugar, 2′-deoxy modified sugar, 2′-F modified sugar, 2′-arabino-fluoro modified sugar, 2′-O-benzyl modified sugar, and 2′-O-methyl-4-pyridine modified sugar.

46-77. (canceled)

78. The oligomeric compound according to claim 1, wherein the modified nucleotide has a nucleobase sequence that consists of first region is selected from the group consisting of SEQ ID NOs: 811 and 911, 780 and 880, 787 and 887, 837 and 937, 805 and 905, 816 and 916, 773 and 873, 833 and 933, 793 and 893, 797 and 897, 822 and 922, 777 and 877, 764 and 864, 778 and 878, 796 and 896, 832 and 932, 824 and 924, 825 and 925, 823 and 923, 803 and 903, 766 and 866, 786 and 886, 809 and 909, 792 and 892, and 806 and 906.

79-81. (canceled)

82. A nucleic acid construct comprising at least:
(a) a first nucleic acid portion that is at least partially complementary to at least a first portion of an RNA, which is transcribed from an C5 gene;
(b) a second nucleic acid portion that is at least partially complementary to at least a second portion of an RNA, which is transcribed from an C5 gene, the second portion being different from the first portion;
(c) a third nucleic acid portion that is at least partially complementary to the first nucleic acid portion of (a), so as to form a first nucleic acid duplex region therewith;
(d) a fourth nucleic acid portion that is at least partially complementary to the second nucleic acid portion of (b), so as to form a second nucleic acid duplex region therewith,
wherein the construct is designed such that subsequent to in vivo administration the construct disassembles to yield at least first and second discrete nucleic acid targeting molecules that respectively target the RNA portions transcribed from the target genes of (a) and (b);
whereby (i) the first nucleic acid targeting molecule is capable of modulating expression of the target gene of (a), and comprises, or is derived from, at least the first nucleic acid portion of (a), and (ii) the second nucleic acid targeting molecule is capable of modulating expression of the target gene of (b), and comprises, or is derived from, the second nucleic acid portion of (b).

83-153. (canceled)

154. The construct of claim 82, wherein
(a) the first nucleic acid portion is selected from the group consisting of SEQ ID Nos. 751-850;
(b) the second nucleic acid portion is selected from the group consisting of SEQ ID Nos. 751-850;
(c) the third nucleic acid portion is selected from the group consisting of SEQ ID Nos. 851-950; and/or
(d) the fourth nucleic acid portion is selected from the group consisting of SEQ ID Nos. 851-950.

155-162. (canceled)

163. A pharmaceutical composition comprising an oligomeric compound according to claim 1 and a pharmaceutically acceptable excipient, diluent, antioxidant, and/or preservative.

164-165. (canceled)

166. The pharmaceutical composition of claim 163, further comprising one or more further pharmaceutically active agents.

167-174. (canceled)

175. A method of treating a C5-associated disease or disorder or a disease requiring reduced C5 expression, comprising administering to a patient suffering from said disease or disorder a therapeutically effective amount of an oligomeric compound according to claim 1, wherein said disease or disorder is selected from the group consisting of autoimmune disease, complement system dysfunction including aberrant upregulation of complement components such as C5, age-related macular degeneration (AMD) including dry AMD and geographic atrophy, paroxysmal nocturnal hemoglobinuria (PNH), Generalized myasthenia gravis (gMG), Lupus nephritis (LN), Alzheimer's disease, Atherosclerosis, Inflammation of the choroid plexus, atypical hemolytic uremic syndrome (aHUS), C3 glomerulopathy (C3G), lg-mediated kidney pathologies such as IgA nephropathy and primary membranous nephropathy, asthma, rheumatic disease, rheumatoid arthritis, systemic lupus erythematosus (SLE), anti-neutrophil cytoplasmic antibody (ANCA) vasculitis, antiphospholipid antibody syndrome (APS), glomerulonephritis, dermatomyositis bullous pemphigoid, Shiga toxin E. coli-related hemolytic uremic syndrome, amyotrophic lateral sclerosis (ALS), Central nervous system (CNS) diseases, myasthenia gravis (MG), neuromyelistis optica (NMO), dense deposit disease, C3 neuropathy, cold agglutinin disease, humoral and vascular transplant rejection, graft dysfunction, myocardial infarction, asthma, rheumatoid arthritis (RA) sensitization towards a transplant, antiphospho lipid antibody syndrome; lupus nephritis; ischemia-reperfusion injury; typical or infectious hemolytic uremic syndrome (tHUS); dense deposit disease (DDD); neuromyelitis optica (N O); multifocal motor neuropathy (MMN); multiple sclerosis (MS); macular degeneration (e.g., age-related macular degeneration (AM_D)); hemolysis, elevated liver enzymes, and low platelets (HELLP) syndrome; thrombotic thrombocytopenia purpura (TTP); spontaneous fetal loss; Pauci-immune vasculitis; epidermolysis bullosa; recurrent fetal loss; pre-eclampsia, traumatic brain injury, myasthenia gravis, cold agglutinin disease, dermatomyositis bullous pemphigoid, Shiga toxin E. co/-related hemolytic uremic syndrome, C3 nephropathy, anti-neutrophil cytoplasmic antibody-associated vasculitis, humoral and vascular transplant rejection, graft dysfunction, myocardial infarction, an allogenic transplant, sepsis, Coronary artery disease, dermatomyositis, Graves' disease, atherosclerosis, systemic inflammatory response sepsis, septic shock, spinal cord injury, glomerulonephritis, Hashimoto's thyroiditis, type I diabetes, pemphigus, autoimmune hemolytic anemia (AIHA), ITP, Goodpasture syndrome, Degos disease, antiphospholipid syndrome (APS), catastrophic APS (CAPS), a cardiovascular disorder, myocarditis, a cerebrovascular disorder, a peripheral vascular disorder, a renovascular disorder, a mesenteric/enteric vascular disorder, vasculitis, Henoch-Schonlein purpura nephritis, systemic lupus erythematosus-associated vasculitis, vasculitis associated with rheumatoid arthritis, immune comple vasculitis, Takayasu's disease, dilated cardiomyopathy, diabetic angiopathy, Kawasaki's disease (arteritis), venous gas embolus (VGE), and restenosis following stent placement, rotational atherectomy, membraneous nephropathy, Guiliain-Barre syndrome, and percutaneous transluminal coronary angioplasty Age-related macular degeneration (AMD) and/or Geographic atrophy (GA); Uveitis and/or panuveitis; Cold agglutinin disease, Membranoproliferative glomerulonephritis (MPGN), Guillain-Barre syndrome, Shiga toxin-producing E. coli hemolytic-uremic syndrome (STEC-HUS), organ transplantation-associated autoimmune diseases, and sepsis.

176-178. (canceled)

179. The oligomeric compound according to claim 33, wherein the single strand is selected from the group consisting of SEQ ID NOs: 980 and 987.
